(19) United States
(12) Patent Application Publication
(10) Pub. No.: US 2006/0199816 A1
(43) Pub. Date: Sep. 7, 2006

(54) Aryl sulfonyl piperidines

(76) Inventors: Paul Gillespie, Westfield, NJ (US); Robert Alan Goodnow JR., Gillette, NJ (US); Agnieszka Kowalczyk, Mine Hill, NJ (US); Sung-Sau So, Nutley, NJ (US); Qiang Zhang, Parsippany, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/365,053

(22) Filed: Mar. 1, 2006

Related U.S. Application Data
(60) Provisional application No. 60/658,276, filed on Mar. 3, 2005.

(51) Int. Cl. A61K 31/538; A61K 31/4709; A61K 31/453; A61K 31/445

(52) U.S. Cl. 514/230.5; 514/314; 514/319; 514/320

(57) ABSTRACT

Provided herein are compounds of the formula (1): 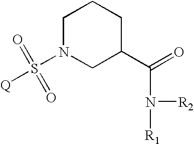 as well as pharmaceutically acceptable salts thereof, wherein the substituents are as those disclosed in the specification. These compounds, and the pharmaceutical compositions containing them, are useful for the treatment of diseases such as, for example, type II diabetes mellitus and metabolic syndrome.

### PRIORITY TO RELATED APPLICATIONS

This application claims the benefit of U.S. Provisional Application No. 60/658,276, filed Mar. 3, 2005, which is hereby incorporated by reference in its entirety.

### FIELD OF THE INVENTION

The invention relates to inhibitors of 11β-hydroxysteroid dehydrogenase. The inhibitors include, for example, aryl sulfonyl piperidines and are useful for the treatment of diseases such as type II diabetes mellitus and metabolic syndrome.

All documents cited or relied upon below are expressly incorporated herein by reference.

### BACKGROUND OF THE INVENTION

Diabetes mellitus is a serious illness that affects an increasing number of people across the world. Its incidence is escalating parallel to the upward trend of obesity in many countries. The serious consequences of diabetes include increased risk of stroke, heart disease, kidney damage, blindness, and amputation.

Diabetes is characterized by decreased insulin secretion and/or an impaired ability of peripheral tissues to respond to insulin, resulting in increased plasma glucose levels. There are two forms of diabetes: insulin-dependent and non-insulin-dependent, with the great majority of diabetics suffering from the non-insulin-dependent form of the disease, known as type 2 diabetes or non-insulin-dependent diabetes mellitus (NIDDM). Because of the serious consequences, there is an urgent need to control diabetes.

Treatment of NIDDM generally starts with weight loss, a healthy diet and an exercise program. These factors are especially important in addressing the increased cardiovascular risks associated with diabetes, but they are generally ineffective in controlling the disease itself. There are a number of drug treatments available, including insulin, metformin, sulfonylureas, acarbose, and thiazolidinediones. However, each of these treatments has disadvantages, and there is an ongoing need for new drugs to treat diabetes.

Metformin is an effective agent that reduces fasting plasma glucose levels and enhances the insulin sensitivity of peripheral tissue. Metformin has a number of effects in vivo, including an increase in the synthesis of glycogen, the polymeric form in which glucose is stored [R. A. De Fronzo Drugs 1999, 58 Suppl. 1, 29]. Metformin also has beneficial effects on lipid profile, with favorable results on cardiovascular health—treatment with metformin leads to reductions in the levels of LDL cholesterol and triglycerides [S. E. Inzucchi JAMA 2002, 287, 360]. However, over a period of years, metformin loses its effectiveness [R. C. Turner et al. JAMA 1999, 281, 2005] and there is consequently a need for new treatments for diabetes.

Thiazolidinediones are activators of the nuclear receptor peroxisome-proliferator activated receptor-gamma. They are effective in reducing blood glucose levels, and their efficacy has been attributed primarily to decreasing insulin resistance in skeletal muscle [M. Tadayyon and S. A. Smith Expert Opin. Investig. Drugs 2003, 12, 307]. One disadvantage associated with the use of thiazolidinediones is weight gain.

Sulfonylureas bind to the sulfonylurea receptor on pancreatic beta cells, stimulate insulin secretion, and consequently reduce blood glucose levels. Weight gain is also associated with the use of sulfonylureas [S. E. Inzucchi JAMA 2002, 287, 360] and, like metformin, they lose efficacy over time [R. C. Turner et al. JAMA 1999, 281, 2005]. A further problem often encountered in patients treated with sulfonylureas is hypoglycemia [M. Salas J. J. and Caro Adv. Drug React. Tox. Rev. 2002, 21, 205-217].

Acarbose is an inhibitor of the enzyme alpha-glucosidase, which breaks down disaccharides and complex carbohydrates in the intestine. It has lower efficacy than metformin or the sulfonylureas, and it causes intestinal discomfort and diarrhea which often lead to the discontinuation of its use [S. E. Inzucchi JAMA 2002, 287, 360]

Because none of these treatments is effective over the long term without serious side effects, there is a need for new drugs for the treatment of type 2 diabetes.

The metabolic syndrome is a condition where patients exhibit more than two of the following symptoms: obesity, hypertriglyceridemia, low levels of HDL-cholesterol, high blood pressure, and elevated fasting glucose levels. This syndrome is often a precursor of type 2 diabetes, and has a high estimated prevalence in the United States of 24% (E. S. Ford et al. JAMA 2002, 287, 356). A therapeutic agent that ameliorates the metabolic syndrome would be useful in potentially slowing or stopping the progression to type 2 diabetes.

In the liver, glucose is produced by two different processes: gluconeogenesis, where new glucose is generated in a series of enzymatic reactions from pyruvate, and glycolysis, where glucose is generated by the breakdown of the polymer glycogen.

Two of the key enzymes in the process of gluconeogenesis are phosphoenolpyruvate carboxykinase (PEPCK) which catalyzes the conversion of oxalacetate to phosphoenolpyruvate, and glucose-6-phosphatase (G6Pase) which catalyzes the hydrolysis of glucose-6-phosphate to give free glucose. The conversion of oxalacetate to phosphoenolpyruvate, catalyzed by PEPCK, is the rate-limiting step in gluconeogenesis. On fasting, both PEPCK and G6Pase are upregulated, allowing the rate of gluconeogenesis to increase. The levels of these enzymes are controlled in part by the corticosteroid hormones (cortisol in human and corticosterone in mouse). When the corticosteroid binds to the corticosteroid receptor, a signaling cascade is triggered which results in the upregulation of these enzymes.

The corticosteroid hormones are found in the body along with their oxidized 11-dehydro counterparts (cortisone and 11-dehydrocorticosterone in human and mouse, respectively), which do not have activity at the glucocorticoid receptor. The actions of the hormone depend on the local concentration in the tissue where the corticosteroid receptors are expressed. This local concentration can differ from the circulating levels of the hormone in plasma, because of the actions of redox enzymes in the tissues. The enzymes that modify the oxidation state of the hormones are 11beta-hydroxysteroid dehydrogenases forms I and II. Form I (11β-HSD1) is responsible for the reduction of cortisone to cortisol in vivo, while form 11 (11β-HSD2) is responsible for the oxidation of cortisol to cortisone. The enzymes have low homology and are expressed in different tissues. 11β-HSD1 is highly expressed in a number of tissues including liver, adipose tissue, and brain, while 11β-HSD2 is highly expressed in mineralocorticoid target tissues, such as kidney and colon. 11β-HSD2 prevents the binding of cortisol to the mineralocorticoid receptor, and defects in this enzyme have been found to be associated with the syndrome of apparent mineralocorticoid excess (AME).

Since the binding of the 11β-hydroxysteroids to the corticosteroid receptor leads to upregulation of PEPCK and therefore to increased blood glucose levels, inhibition of 11β-HSD1 is a promising approach for the treatment of diabetes. In addition to the biochemical discussion above, there is evidence from transgenic mice, and also from small clinical studies in humans, that confirm the therapeutic potential of the inhibition of 11β-HSD1.

Experiments with transgenic mice indicate that modulation of the activity of 11β-HSD1 could have beneficial therapeutic effects in diabetes and in the metabolic syndrome. For example, when the 11β-HSD1 gene is knocked out in mice, fasting does not lead to the normal increase in levels of G6Pase and PEPCK, and the animals are not susceptible to stress- or obesity-related hyperglycemia. Moreover, knockout animals which are rendered obese on a high-fat diet have significantly lower fasting glucose levels than weight-matched controls (Y. Kotolevtsev et al. Proc. Natl. Acad Sci. USA 1997, 94, 14924). 11β-HSD1 knockout mice have also been found to have improved lipid profile, insulin sensitivity, and glucose tolerance (N. M. Morton et al. J. Biol. Chem. 2001, 276, 41293). The effect of overexpressing the 11β-HSD1 gene in mice has also been studied. These transgenic mice displayed increased 11β-HSD1 activity in adipose tissue, and they also exhibit visceral obesity which is associated with the metabolic syndrome. Levels of the corticosterone were increased in adipose tissue, but not in serum, and the mice had increased levels of obesity, especially when on a high-fat diet. Mice fed on low-fat diets were hyperglycemic and hyperinsulinemic, and also showed glucose intolerance and insulin resistance (H. Masuzaki et al. Science, 2001, 294, 2166).

The effects of the non-selective 11β-hydroxysteroid dehydrogenase inhibitor carbenoxolone have been studied in a number of small trials in humans. In one study, carbenoxolone was found to lead to an increase in whole body insulin sensitivity, and this increase was attributed to a decrease in hepatic glucose production (B. R. Walker et al. J. Clin. Endocrinol. Metab. 1995, 80, 3155). In another study, decreased glucose production and glycogenolysis in response to glucagon challenge were observed in diabetic but not healthy subjects (R. C. Andrews et al. J. Clin. Enocrinol. Metab. 2003, 88, 285). Finally, carbenoxolone was found to improve cognitive function in healthy elderly men and also in type 2 diabetics (T. C. Sandeep et al. Proc. Natl. Acad. Sci USA 2004, 101, 6734).

A number of non-specific inhibitors of 11β-HSD1 and 11β-HSD2 have been identified, including glycyrrhetinic acid, abietic acid, and carbenoxolone. In addition, a number of selective inhibitors of 11β-HSD1 have been found, including chenodeoxycholic acid, flavanone and 2′-hydroxyflavanone (S. Diederich et al. Eur. J. Endocrinol. 2000, 142, 200 and R. A. S. Schweizer et al. Mol. Cell. Endocrinol. 2003, 212, 41).

WO 2004089470, WO 2004089416 and WO 2004089415 (Novo Nordisk A/S) disclose compounds with a number of different structural types as inhibitors of 11bHSD1 useful for the treatment of metabolic syndrome and related diseases and disorders.

WO 0190090, WO 0190091, WO 0190092, WO 0190093, WO 03043999 (Biovitrum AB) disclose compounds as inhibitors of 11β-HSD1. These compounds are different in structure to the compounds of the current invention. WO 2004112781 and WO 2004112782 disclose the method of use of some of these compounds for the promotion of wound healing.

WO 0190094, WO 03044000, WO 03044009, and WO 2004103980 (Biovitrum AB) disclose compounds as inhibitors of 11β-HSD1. These compounds are different in structure to the compounds of the current invention. WO 2004112785 discloses the method of use of some of these compounds for the promotion of wound healing.

WO 03065983, WO 03075660, WO 03104208, WO 03104207, US2004013301 1, WO 2004058741, and WO 2004106294 (Merck & Co., Inc.) disclose compounds as inhibitors of 11β-HSD1. These compounds are different in structure to the compounds of the current invention. US2004122033 discloses the combination of an appetite suppressant with inhibitors of 11β-HSD1 for the treatment of obesity, and obesity-related disorders.

WO 2004065351 (Novartis); WO 2004056744 and WO 2004056745 (Janssen Pharmaceutica N. V.); and WO 2004089367 and WO 2004089380 (Novo Nordisk A/S) discloses compounds as inhibitors of 11β-HSD1. These compounds are different in structure to the compounds of the current invention.

WO 2004089415 (Novo Nordisk A/S) discloses the use of an inhibitor of 11β-HSD1 in combination with an agonist of the glucocorticoid receptor for the treatment of diseases including cancer and diseases involving inflammation. Several different classes of 11β-HSD1 inhibitors are disclosed including amino-ketones, benzimidazoles, carboxamides, 2,3-dihydrobenzofuran-7-carboxamides, indoles, methylenedioxyphenyl-carboxamides, oxazole-4-carboxamides, oxazole-5-carboxamides pyrazolo[1,5-a]pyrimidines, pyrazole-4-carboxamides, thiazole-4-carboxamides, thiazole-5-carboxamides, and 1,2,4-triazoles. WO 2004089416 (Novo Nordisk A/S) discloses the use of an inhibitor of 11β-HSD1 in combination with an antihypertensive agent for the treatment of diseases including insulin resistance, dyslipidemia and obesity. WO 2004089470 (Novo Nordisk A/S) discloses substituted amides as inhibitors of 11β-HSD1.

WO 2004089471 (Novo Nordisk A/S) discloses pyrazolo[1,5-a]pyrimidines as inhibitors of 11β-HSD1; WO 2004089896 (Novo Nordisk A/S) discloses compounds as inhibitors of 11β-HSD1; WO 2004037251A1 (Sterix Limited) discloses sulfonamides as inhibitors of 11β-HSD1; WO 2004027047A2 (Hartmut Hanauske-Abel) discloses compounds as inhibitors of 11β-HSD1; and WO 2004011410, WO 2004033427, and WO 2004041264 (AstraZeneca UK Limited) disclose compounds as inhibitors of 11β-HSD1. These compounds are different in structure to the compounds of the current invention.

WO 02076435A2 (The University of Edinburgh) claims the use of an agent which lowers levels of 11β-HSD1 in the manufacture of a composition for the promotion of an atheroprotective lipid profile. Agents mentioned as inhibitors of 11β-HSD1 include carbenoxolone, 11-oxoprogesterone, 3α,17,21-trihydroxy-5β-pregnan-3-one, 21-hydroxy-pregn-4-ene-3,11,20-trione, androst-4-ene-3,11,20-trione and 3β-hydroxyandrost-5-en-17-one. None of these compounds is similar in structure to the compounds of the current invention.

WO 03059267 (Rhode Island Hospital) claims a method for treating a glucocorticoid-associated state by the administration of a 11β-HSD1 inhibitor such as 11-ketotestosterone, 11-keto-androsterone, 11-keto-pregnenolone, 11-keto-dehydro-epiandrostenedione, 3α,5α-reduced-11-ketoprogesterone, 3α,5α-reduced-11-ketotestosterone, 3α,5α-reduced-11-keto-androstenedione, or 3α,5α-tetrahydro-11β-dehydro-corticosterone. None of these compounds is similar in structure to the compounds of the current invention.

WO 9610022 (Zeneca Limited) discloses 1-[[1-(2-naphthalenylsulfonyl)-3-piperidinyl]carbonyl]-4-(4-pyridinyl)-piperazine as an antithrombotic or anticoagulant agent.

WO 2004018428 (Pharmacia & Upjohn) discloses 5-cyano-2-[[[4-[[3-[(diethylamino)carbonyl]-1-piperidinyl]sulfonyl]-5-methyl-2-thienyl]carbonyl]amino]-benzoic acid as an antibacterial agent

WO 2004018414 (Pharmacia & Upjohn) discloses 5-cyano-2-[[3-[[3-[(diethylamino)carbonyl]-1-piperidinyl]sulfonyl]benzoyl]amino]-benzoic acid and 5-cyano-2-[[4-[[3-[(diethylamino)carbonyl]-1-piperidinyl]sulfonyl]benzoyl]amino]-benzoic acid as antibacterial agents

WO 2002020015 (Merck & Co., Inc.) discloses N-[(1R)-1-(4-cyano-3-fluorophenyl)-1-(1-methyl-1H-imidazol-5-yl)ethyl]-1-[(3-methoxyphenyl)sulfonyl]-3-piperidinecarboxamide and N-[(1R)-1-(4-cyano-3-fluorophenyl)-1-(1-methyl-1H-imidazol-5-yl)ethyl]-1-[(3-hydroxyphenyl)sulfonyl]-3-piperidinecarboxamide as intermediates in the preparation of macrocyclic inhibitors of prenyl-protein transferase.

US 2004029883 (Bayer, A. G., Germany) discloses compounds as inhibitors of inflammatory, autoimmune and immune diseases. These compounds are different in structure to the compounds of the current invention.

GB 2351733 and C. Zhou et al. Bioorg. Med. Chem. Lett. 2001, 11, 415 disclose (βS)—N-[[1-[(4-fluorophenyl)sulfonyl]-3-piperidinyl]carbonyl]-β-methyl-D-tryptophyl-L-Lysine, 1,1-dimethylethyl ester, monoacetate, (βS)—N-[[1-[(3,4-dimethoxyphenyl)sulfonyl]-3-piperidinyl]carbonyl]-β-methyl-D-tryptophyl-L-Lysine, 1,1-dimethylethyl ester, and (βS)-β-methyl-N-[[1-(2-thienylsulfonyl)-3-piperidinyl]carbonyl]-D-tryptophyl-L-Lysine, 1,1-dimethylethyl ester as somatostatin receptor 2 agonists for the treatment and prevention of diabetes, cancer, acromegaly, depression, chronic atrophic gastritis, Crohn's disease, ulcerative colitis, retinopathy, arthritis, pain both visceral and neuropathic and to prevent restenosis. These compounds are different in structure to the compounds of the current invention.

WO 2001012186 (Biogen, Inc.) discloses (2S)-4-[[(2S)-4-methyl-2-[methyl[[4-[[[2-methylphenyl)amino]carbonyl]amino]phenyl]acetyl]amino]-1-oxopentyl]amino]-2-[[[(3S)-1-(phenylsulfonyl)-3-piperidinyl]carbonyl]amino]-butanoic acid as a cell adhesion inhibitor. This compound is different in structure to the compounds of the current invention.

WO 2001007440 (Boehringer Ingelheim Pharmaceuticlas, Inc.) discloses 1-[[(3R)-3-[(4-bromophenyl)methyl]-1-(3,5-dichlorophenyl)-2,3-dihydro-3-methyl-2-oxo-1H-imidazo[1,2-a]imidazol-5-yl]sulfonyl]-N,N-diethyl-3-piperidinecarboxamide as an anti-inflammatory agent.

WO 2000048623 (Kaken Pharmaceutical Co., Ltd) discloses N-[(1R)-2-[(3-aminopropyl)amino]-1-(2-naphthalenylmethyl)-2-oxoethyl]-1-(phenylsulfonyl)-3-piperidinecarboxamide, monohydrochloride (9CI) as a growth hormone.

U.S. Pat. No. 5,817,678 (Merck & Co., Inc.) discloses (3S)—N-[2-[1-[(4-cyanophenyl)methyl]-1H-imidazol-5-yl]ethyl]-1-(phenylsulfonyl)-3-piperidinecarboxamide, (3S)—N-[2-[1-[(4-cyanophenyl)methyl]-1H-imidazol-5-yl]ethyl]-1-(naphthalenesulfonyl)-3-piperidinecarboxamide, (3S)-1-[(3-chlorophenyl)sulfonyl]-N-[2-[1-[(4-cyanophenyl)methyl]-1H-imidazol-5-yl]ethyl]-3-piperidinecarboxamide, and (3S)—N-[2-[1-[(4-cyanophenyl)methyl]-1H-imidazol-5-yl]ethyl]-1-[(3,5-dichlorophenyl)sulfonyl]-3-piperidinecarboxamide as farnesyl-protein transferase inhibitors.

WO 9910523, WO 9910524, WO 9910525 and WO 2000016626 (Merck & Co., Inc.) also disclose (3S)—N-[2-[1-[(4-cyanophenyl)methyl]-1H-imidazol-5-yl]ethyl]-1-[(3,5-dichlorophenyl)sulfonyl]-3-piperidinecarboxamide as an inhibitor of prenyl protein transferases for cancer treatment.

Scozzafava et al. Eur. J. Med. Chem. 2000, 35, 31 discloses N-[2-(1H-imidazol-4-yl)ethyl]-1-[(4-methylphenyl)sulfonyl]-3-piperidinecarboxamide as an activator of carbonic anhydrase isoenzymes I, II and IV.

DE 19827640 (Bayer A.-G.) discloses 1-[[3-(7-cyclopentyl-1,4-dihydro-5-methyl-4-oxoimidazo[5,1-f][1,2,4]triazin-2-yl)-4-ethoxyphenyl]sulfonyl]-N,N-diethyl-3-piperidinecarboxamide, 1-[[3-(7-cycloheptyl-1,4-dihydro-5-methyl-4-oxoimidazo[5,1-f][1,2,4]triazin-2-yl)-4-ethoxyphenyl]sulfonyl]-N,N-diethyl-3-piperidinecarboxamide, and, 1-[[4-ethoxy-3-(7-hexyl-1,4-dihydro-5-methyl-4-oxoimidazo[5,1-f][1,2,4]triazin-2-yl)phenyl]sulfonyl]-N,N-diethyl-3-piperidinecarboxamide as phosphodiesterase inhibitors

WO 9964004 (Bristol-Myers Squibb Company) discloses 1-[[1-[[3-(5,8-dihydro-8-oxo-1H-imidazo[4,5-g]quinazolin-6-yl)-4-propoxyphenyl]sulfonyl]-3-piperidinyl]carbonyl]-4-methyl-piperazine as an inhibitor of cGMP phosphodiesterase.

A need exits in the art, however, for additional 11β-HSD1 inhibitors that have efficacy for the treatment of diseases such as type II diabetes mellitus and metabolic syndrome. Further, a need exists in the art for 11β-HSD1 inhibitors having IC50 values less than about 1 μM.

### SUMMARY OF THE INVENTION

In one embodiment of the present invention, a pharmaceutical composition comprising a therapeutically effective amount of a compound according to formula (I) is provided: 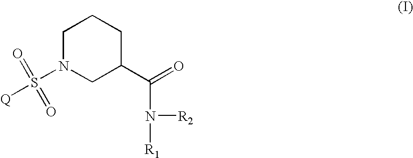
wherein
Q is unsubstituted phenyl,
substituted phenyl which is phenyl mono-, di-, or tri-substituted with a group independently selected from the group consisting of halogen, lower alkyl, —COOA, —CF3,—OA, —NC(═O)A, and phenyl,
unsubstituted heterocyclyl which is a 5- or 6-membered heteroaromatic ring which is connected by a ring carbon atom and which has from 1 to 3 hetero ring atoms selected from the group consisting of sulfur, nitrogen and oxygen,
substituted heterocyclyl which is heterocyclyl which is substituted with —COOA or halogen, naphthyl,
9- and 10-membered bicyclic unsaturated or partially unsaturated heterocyclyl which is connected by a ring carbon and which has from 1 to 3 hetero ring atoms selected from the group consisting of sulfur, nitrogen and oxygen,
substituted bicyclic heterocyclyl which is the 9- or 1 0-membered bicyclic heterocyclyl mono-,
   bi- or tri-substituted with substituents selected from halogen or lower alkyl;
one of R₁ or R₂ is H and the other is selected from the group consisting of
lower alkyl,
a mono-substituted or unsubstituted saturated mono-, bi- or tri-cyclic 5 to 10 membered carbocyclic ring, wherein the mono-substituted carbocyclic ring is substituted with lower alkyl,
a bicyclic partially unsaturated 9- or 10-membered ring,
—CH2B,
-D-phenyl or D-substituted phenyl, wherein D-substituted phenyl is D-phenyl in which the phenyl is mono- or di-substituted with —OA, halogen, or substituted or unsubstituted lower alkyl
-D-naphthyl,
-DE,
-DN(CH₃)n-phenyl,
-DNC(═O)A,
-DN(A)A,
-DOA; or
R₁ and R₂, together with the N atom to which they are attached, form a substituted or unsubstituted ring Z, wherein Z is 6- or 7-membered monocyclic or 7- to 10-membered bicyclic saturated, partially unsaturated or unsaturated substituted or unsubstituted heterocyclic ring which contains the N atom to which R₁ and R₂ are attached, and optionally another hetero atom which is selected from N, O and S, wherein the substituted heterocyclic ring is mono- or di-substituted with lower alkyl or hydroxy or hydroxy-alkyl;
A is lower alkyl which has from 1 to 4 carbon atoms,
B is a 3- to 7-membered substituted or unsubstituted carbocyclic saturated ring,
D is the divalent form of A,
E is a 5- or 6-membered saturated, unsaturated or partially unsaturated heterocyclic ring having from 1 to 3 hetero atoms selected from the group consisting of S, N, and O,
n is zero or 1,
provided that where R₁ or R₂ is H and the other is lower alkyl, and where Q is monosubstituted in the para position with halogen, then the halogen is chloro,
provided that where R₁ or R₂ is H and the other is lower alkyl, and where Q is monosubstituted in the para position with lower alkyl, then the lower alkyl has from 1 to 3 carbon atoms,
provided that where R₁ or R₂ is H and the other is CH2B, and where Q is substituted phenyl wherein the phenyl ring is monosubstituted in the meta position with halogen, the halogen is not C₁,
provided that where R₁ or R₂ is H and the other is D-substituted phenyl in which D is —CH₂CH₂— and the phenyl is monosubstituted in the ortho position with F, and where Q is substituted phenyl wherein phenyl is monosubstituted with halogen, the halogen is not Cl in the meta position,
provided that where R₁ or R² is H and the other is -D-substituted phenyl in which D is —CH₂— and the phenyl is monosubstituted with lower alkyl which is —CH3 in the ortho position and where Q is substituted phenyl which is phenyl substituted with halogen, the halogen is not C₁ in the ortho position,
or a pharmaceutically acceptable salt thereof,
and a pharmaceutically acceptable carrier.

In another embodiment of the present invention, a method for the treatment of type II diabetes in a patient in need thereof is provided, comprising administering to said patient a therapeutically effective amount of a compound according to formula (I).

### DETAILED DESCRIPTION OF THE INVENTION

The present invention pertains to inhibitors of 11β-HSD1. In a preferred embodiment, the invention provides for pharmaceutical compositions comprising sulfonyl piperidines of the formula I: 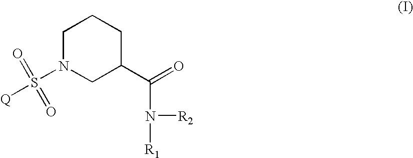
as well as pharmaceutically acceptable salts thereof, that are useful as inhibitors of 11β-HSD1.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

In this specification the term “aryl” is used to mean a mono- or polycyclic aromatic ring system, in which the rings may be carbocyclic or may contain one or more atoms selected from O, S, and N. Examples of aryl groups are phenyl, pyridyl, benzimidazolyl, benzofuranyl, benzothiazolyl, benzothiophenyl, cinnolinyl, furyl, imidazo[4,5-c]pyridinyl, imidazolyl, indolyl, isoquinolinyl, isoxazolyl, naphthyl, [1,7]naphthyridinyl, oxadiazolyl, oxazolyl, phthalazinyl, purinyl, pyidazinyl, pyrazolyl, pyrido[2,3-d]pyrimidinyl, pyrimidinyl, pyrimido[3,2-c]pyrimidinyl, pyrrolo[2,3-d]pyrimidinyl, pyrrolyl, quinazolinyl, quinolinyl, quinoxalinyl, tetrazolyl, thiadiazolyl, thiazolyl, thiophenyl, triazolyl, and the like.

As used herein, the term “alkyl” means, for example, a branched or unbranched, cyclic or acyclic, saturated or unsaturated (e.g. alkenyl or alkynyl) hydrocarbyl radical which may be substituted or unsubstituted. Where cyclic, the alkyl group is preferably C₃ to C₁₂, more preferably C₅ to C₁₀, more preferably C₅ to C₇. Where acyclic, the alkyl group is preferably C₁ to C₁₀, more preferably C₁ to C₆, more preferably methyl, ethyl, propyl (n-propyl or isopropyl), butyl (n-butyl, isobutyl or tertiary-butyl) or pentyl (including n-pentyl and isopentyl), more preferably methyl. It will be appreciated therefore that the term “alkyl” as used herein includes alkyl (branched or unbranched), substituted alkyl (branched or unbranched), alkenyl (branched or unbranched), substituted alkenyl (branched or unbranched), alkynyl (branched or unbranched), substituted alkynyl (branched or unbranched), cycloalkyl, substituted cycloalkyl, cycloalkenyl, substituted cycloalkenyl, cycloalkynyl and substituted cycloalkynyl.

As used herein, the term “lower alkyl” means, for example, a branched or unbranched, cyclic or acyclic, saturated or unsaturated (e.g. alkenyl or alkynyl) hydrocarbyl radical wherein said cyclic lower alkyl group is C₅, C₆ or C₇, and wherein said acyclic lower alkyl group is C₁, C₂, C₃ or C₄, and is preferably selected from methyl, ethyl, propyl (n-propyl or isopropyl) or butyl (n-butyl, sec-butyl, isobutyl or tertiary-butyl). It will be appreciated therefore that the term “lower alkyl” as used herein includes lower alkyl (branched or unbranched), lower alkenyl (branched or unbranched), lower alkynyl (branched or unbranched), cycloloweralkyl, cycloloweralkenyl and cycloloweralkynyl.

The alkyl and aryl groups may be substituted or unsubstituted. Where substituted, there will generally be, for example, 1 to 3 substituents present, preferably 1 substituent. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters(e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono-or di-alkylaminocarbonylalkyl, arylaminocarbonyl), carbamates (e.g. alkoxycarbonylamino, arloxycarbonylamino, aminocarbonyloxy, mono-or di-alkylaminocarbonyloxy, arylaminocarbonyloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more, preferably one, heteroatom, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

The lower alkyl groups may be substituted or unsubstituted, preferably unsubstituted. Where substituted, there will generally be, for example, 1 to 3 substitutents present, preferably 1 substituent.

As used herein, the term “alkoxy” means, for example, alkyl-O— and “alkoyl” means, for example, alkyl-CO—. Alkoxy substituent groups or alkoxy-containing substituent groups may be substituted by, for example, one or more alkyl groups.

As used herein, the term “halogen” means, for example, a fluorine, chlorine, bromine or iodine radical, preferably a fluorine, chlorine or bromine radical, and more preferably a fluorine or chlorine radical.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminum salts.

### General Synthesis of Compounds According to the Invention

The compounds of the present invention can be prepared by any conventional means. Suitable processes for synthesizing these compounds are provided in the examples. Generally, compounds of formula I can be prepared according to Scheme 1, Scheme 2 or Scheme 3 (see below). The sources of the starting materials for these reactions are also described.

### Preparation of Compounds of the Invention According to Scheme 1

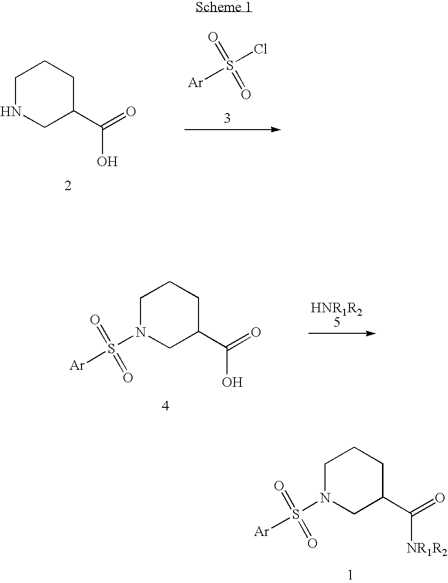

Compounds of formula 1 can be prepared from nipecotic acid (2) according to Scheme 1 by sulfonylation to give a sulfonamide of formula 4 followed by an amide coupling reaction to give the compound of formula 1. The first reaction can be carried out by reacting the compound of formula 2 with a sulfonyl chloride of formula 3 in an inert solvent such as a halogenated hydrocarbon (such as methylene chloride) or an ether (such as tetrahydrofuran or dioxane) or an ester solvent such as ethyl acetate. The reaction is conveniently carried out in the presence of an organic base (such as triethylamine or diisopropylethylamine) or an inorganic base (such as sodium hydroxide or sodium carbonate). When an inorganic base is used, the reaction is conveniently carried out in the additional presence of water, and the co-solvent should be stable to the aqueous base. The reaction can be carried out at a temperature between about 0 degrees and about room temperature, preferably at around room temperature.

Additionally, a number of aryl-sulfonyl-nipecotic acid derivatives of formula 4 are available commercially, and some of these are shown in the table:

| Name | Supplier |
|---|---|
| 1-[(2,4,6-Trimethylphenyl)sulfonyl]- | AsInEx, Moscow, Russia |
| 3-piperidinecarboxylic acid | |
| 1-[(2-Nitrophenyl)sulfonyl]-3- | Ambinter, Paris, France |
| piperidinecarboxylic acid | |
| 1-[(4-Bromophenyl)sulfonyl]-3- | Interchim, Montlucon, France |
| piperidinecarboxylic acid | |
| 1-[(4-Ethoxyphenyl)sulfonyl]-3- | Enamine, Kiev, Ukraine |
| piperidinecarboxylic acid | |
| 1-[(4-Fluorophenyl)sulfonyl]-3- | Interchim, Montlucon, France |
| piperidinecarboxylic acid | |
| 1-[(4-Methoxyphenyl)sulfonyl]-3- | ChemDiv, San Diego, USA |
| piperidinecarboxylic acid | |
| 1-[(4-Methylphenyl)sulfonyl]-3- | AKos Consulting, Basel, |
| piperidinecarboxylic acid | Switzerland |
| 1-[(4-Nitrophenyl)sulfonyl]-3- | Interchim, Montlucon, France |
| piperidinecarboxylic acid | |
| 1-[[4-(Acetylamino)phenyl]sulfonyl]- | Enamine, Kiev, Ukraine |
| 3-piperidinecarboxylic acid | |

The coupling of carboxylic acids of formula 4 with amines of formula 5, according to Scheme 1, can be achieved using methods well known to one of ordinary skill in the art. For example, the transformation can be carried out by reaction of carboxylic acids of formula 4 or of appropriate derivatives thereof such as activated esters, with amines of formula 5 or their corresponding acid addition salts (e.g., the hydrochloride salts) in the presence, if necessary, of a coupling agent, many examples of which are well known per se in peptide chemistry. The reaction is conveniently carried out by treating the carboxylic acid of formula 4 with the hydrochloride of the amine of formula 5 in the presence of an appropriate base, such as diisopropylethylamine, a coupling agent such as O-(benzotriazol-1-yl)-1,1,3,3-tetramethyluronium hexafluorophosphate, and in the optional additional presence of a substance that increases the rate of the reaction, such as 1-hydroxybenzotriazole or 1-hydroxy-7-azabenzotriazole, in an inert solvent, such as a chlorinated hydrocarbon (e.g., dichloromethane) or N,N-dimethylformamide or N-methylpyrrolidinone, at a temperature between about 0 degrees and about room temperature, preferably at about room temperature. Alternatively, the reaction can be carried out by converting the carboxylic acid of formula 4 to an activated ester derivative, such as the N-hydroxysuccinimide ester, and subsequently reacting this with the amine of formula 5 or a corresponding acid addition salt. This reaction sequence can be carried out by reacting the carboxylic acid of formula 4 with N-hydroxysuccinimide in the presence of a coupling agent such as N,N′-dicyclohexylcarbodiimide in an inert solvent such as tetrahydrofuran at a temperature between about 0 degrees and about room temperature. The resulting N-hydroxysuccinimide ester is then treated with the amine of formula 5 or a corresponding acid addition salt, in the presence of a base, such as organic base (e.g., triethylamine or diisopropylethylamine or the like) in a suitable inert solvent such as N,N-dimethylformamide at around room temperature.

### Preparation of Compounds of the Invention According to Scheme 2

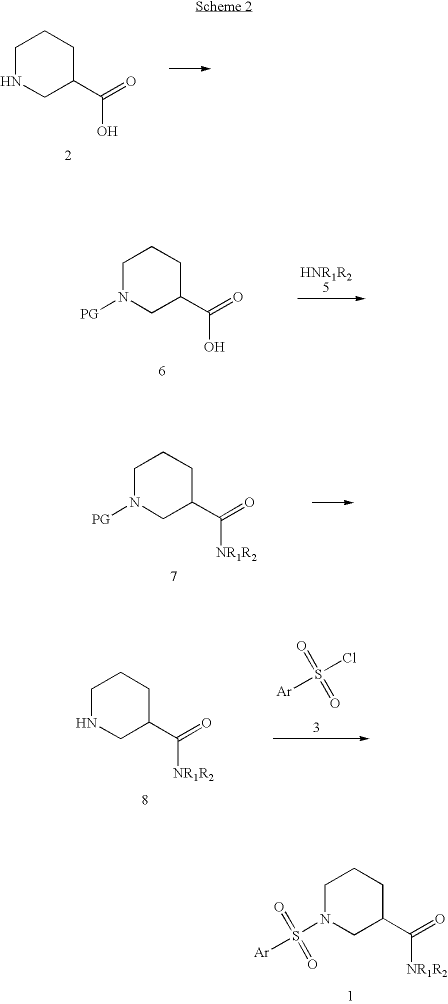

Compounds of the invention of formula 1 can also be prepared according to Scheme 2, which differs from Scheme 1 in the order of the incorporation of the aryl-sulfonyl and amine groups into the molecule. In this process, the nitrogen of the compound of formula 2 is protected to give a compound of formula 6 where PG represents a protective group, many appropriate examples of which are known to one of skill in the art, as discussed below. The compound of formula 6 is then converted to an amide of formula 7, the protective group is then cleaved to give an amine of formula 8 and this compound is then reacted with a sulfonyl chloride of formula 3 to give the compound of formula 1. It will be readily apparent to one of skill in the art that Scheme 2 affords the possibility to prepare compounds of the invention in which one of R¹ or R² represents hydrogen on solid-phase by using a resin-bound amine 5.

Many protective groups PG are known to those of skill in the art of organic synthesis. For example, several suitable protective groups are enumerated in “Protective Groups in Organic Synthesis” [Greene, T. W. and Wuts, P. G. M., 2nd Edition, John Wiley & Sons, N.Y. 1991]. Preferred protective groups are those compatible with the reaction conditions used to prepare compounds of the invention. Examples of such protective groups are tert-butoxycarbonyl (Boc), benzyloxycarbonyl (Cbz), and 9-fluorenylmethoxycarbonyl (Fmoc).

Some examples of intermediates of formula 6 are available commercially, as shown in the table below. Further examples of intermediates of formula 6 can be prepared as described in the subsequent paragraph.

| Compound Name | Supplier |
|---|---|
| (3R)-1-(9-Fluorenylmethoxycarbonyl)-3- | Fluka Chemical Corp., |
| piperidinecarboxylic acid | Milwaukee, WI |
| (3R)-1-(tert-Butoxycarbonyl)-3- | Fluka Chemical Corp., |
| piperidinecarboxylic acid | Milwaukee, WI |
| (3S)-1-(tert-Butoxycarbonyl)-3- | Digital Specialty Chemicals, |
| piperidinecarboxylic acid | Dublin, NH |
| 1-(9-Fuorenylmethoxycarbonyl)-3- | Fluka Chemical Corp., |
| piperidinecarboxylic acid | Milwaukee, WI |
| 1-(tert-Butoxycarbonyl)-3- | Aldrich Chemical Company, |
| piperidinecarboxylic acid | Milwaukee, WI |
| 1-[(Benzyloxy)carbonyl]-3- | Maybridge plc, Tintagel, |
| piperidinecarboxylic acid | Cornwall, UK |

Intermediates of formula 6 can be prepared by reacting the compound of formula 2 with an alkoxycarbonylating reagent such as di-tert-butyl dicarbonate, 2-(tert-butoxycarbonyloxyimino)-2-phenylacetonitrile, benzyl chloroformate, 9-fluorenylmethyl pentafluorophenyl carbonate, N-(9-fluorenylmethoxycarbonyloxy)succinimide, or the like, in the presence of a base which may be organic (for example, triethylamine) or inorganic (for example, sodium hydroxide, sodium or potassium carbonate, or sodium hydrogen carbonate) in an inert solvent such as water or dioxane or tetrahydrofuran, or in a mixture of inert solvents such as a mixture of water and acetone, water and dioxane, or water and tetrahydrofuran. The reaction is conveniently carried out at a temperature between about 0 degrees and about room temperature, preferably at about room temperature. Where the intermediate of formula 6 is not stable to basic conditions, as in the case of a compound of formula 6 in which PG represents Fmoc (9-fluorenylmethoxycarbonyl), care should be taken that this intermediate is not exposed to strongly basic conditions during attempts to prepare it. It will be readily apparent to one of skill in the art that the selection of protective group depends on the nature of the target compound 1, so that for example, the functionalities present in the NR1R2 moiety are compatible with the conditions used to accomplish the removal of the protective group in the conversion of the compound of formula 7 to the compound of formula 8. Because there exist a number of different choices for the protective group PG, with complementary methods of deprotection, there is no difficulty in selecting a protective group for the synthesis of any of the compounds of the invention according to Scheme 2.

The coupling of a carboxylic acid of formula 6 with an amine of formula 5, according to Scheme 2, can be achieved using methods well known to one of ordinary skill in the art. For example, the transformation can be carried out by reaction of a carboxylic acid of formula 6 or of an appropriate derivative thereof such as an activated ester, with an amine of formula 5 or its corresponding acid addition salt (e.g., the hydrochloride salt) in the presence, if necessary, of a coupling agent, many examples of which are well known per se in peptide chemistry. The reaction is conveniently carried out by treating the carboxylic acid of formula 6 with the hydrochloride of the amine of formula 5 in the presence of an appropriate base, such as diisopropylethylamine, a coupling agent such as O-(benzotriazol-1-yl)-1,1,3,3-tetramethyluronium hexafluorophosphate, and in the optional additional presence of a substance that increases the rate of the reaction, such as 1-hydroxybenzotriazole or 1-hydroxy-7-azabenzotriazole, in an inert solvent, such as a chlorinated hydrocarbon (e.g., dichloromethane) or N,N-dimethylformamide or N-methylpyrrolidinone, at a temperature between about 0 degrees and about room temperature, preferably at about room temperature. Alternatively, the reaction can be carried out by converting the carboxylic acid of formula 6 to an activated ester derivative, such as the N-hydroxysuccinimide ester, and subsequently reacting this with the amine of formula 5 or a corresponding acid addition salt. This reaction sequence can be carried out by reacting the carboxylic acid of formula 6 with N-hydroxysuccinimide in the presence of a coupling agent such as N,N′-dicyclohexylcarbodiimide in an inert solvent such as tetrahydrofuran at a temperature between about 0 degrees and about room temperature. The resulting N-hydroxysuccinimide ester is then treated with the amine of formula 5 or a corresponding acid addition salt, in the presence of a base, such as organic base (e.g., triethylamine or diisopropylethylamine or the like) in a suitable inert solvent such as N,N-dimethylformamide at around room temperature.

The removal of the protective group in the conversion of the compound of formula 7 to the amine of formula 8 is carried out according to procedures that are well known in the arts of synthetic chemistry and peptide chemistry and which depend on the nature of the protective group PG. Many examples of suitable procedures are listed in “Protective Groups in Organic Synthesis” [Greene, T. W. and Wuts, P. G. M., 2nd Edition, John Wiley & Sons, N.Y. 1991]. For example, in the case where the protective group is Fmoc (9-fluorenylmethoxycarbonyl), the group can be conveniently removed by treating the compound of formula 7 with an organic base (such as piperidine, morpholine, or ethanolamine) in an inert solvent such as N,N-dimethylformamide or dichloromethane at about room temperature. In the case where the protective group is benzyloxycarbonyl (Cbz), the group can be removed under hydrogenolytic conditions, for example by hydrogenation in the presence of a noble metal catalyst such as palladium-on-carbon, or palladium black, in the presence of an inert solvent (for example, an alcohol such as ethanol) at about room temperature and under atmospheric pressure, or at elevated pressure (such as 50 PSI of hydrogen) if required. As a further example, in the case where the protective group is tert-butoxycarbonyl (Boc), the group can be removed by treatment of the compound of formula 7 with acid (either organic or inorganic) in an inert solvent. For example, the Boc group can be removed by treatment of the compound of formula 7 with trifluoroacetic acid in dichloromethane at about room temperature, or it can be removed by treatment of the compound of formula 7 with hydrochloric acid in an alcoholic solvent (e.g., methanol or ethanol) or an ether (e.g., dioxane) or ethyl acetate, also at about room temperature.

The compound of formula 8 is conveniently converted to the compound of the invention of formula 1 by sulfonylation with a sulfonylating reagent of formula 3. The reaction can be carried out by reacting the compound of formula 8 with a sulfonyl chloride of formula 3 in an inert solvent such as a halogenated hydrocarbon (such as methylene chloride) or an ether (such as tetrahydrofuran or dioxane) or an ester solvent such as ethyl acetate. The reaction is conveniently carried out in the presence of an organic base (such as triethylamine or diisopropylethylamine) or an inorganic base (such as sodium hydroxide or sodium carbonate). When an inorganic base is used, the reaction is conveniently carried out in the additional presence of water, and the co-solvent should be stable to the aqueous base. The reaction can be carried out at a temperature between about 0 degrees and about room temperature, preferably at around room temperature. Many sulfonyl chlorides of formula 3 are commercially available, or can be synthesized according to the many different processes as discussed above.

In the case where a resin-bound amine of formula 5 was used, an additional step is required for the conversion of the resin-bound compound of formula 1 into the compound of the invention; namely, the compound of the invention must be cleaved from the resin. This can be done using any conventional conditions, many of which are known to one of skill in the art of solid-phase organic synthesis, and which conditions will depend on the nature of the linker attaching the product to the solid support. For example, in the case where FMBP resin was used, the cleavage is conveniently effected by treating the resin-bound compound of formula 1 with an organic acid, preferably trifluoroacetic acid, in an inert solvent such as dichloromethane at room temperature.

### Preparation of Compounds of the Invention According to Scheme 3

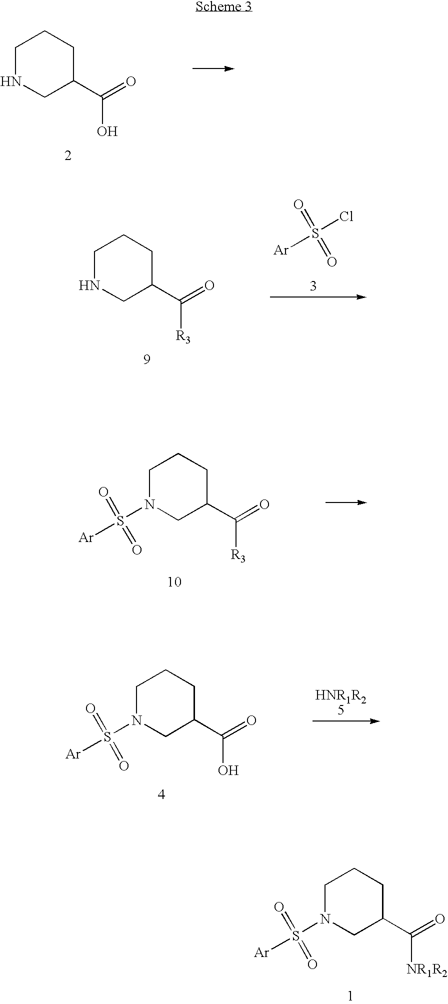

Compounds of the invention of formula 1 can also be prepared according to Scheme 3, which differs from Scheme 1 in that there are an additional two steps in the sequence—a protection step and a deprotection step. In this process, the carboxyl group of the compound of formula 2 is protected to give a compound of formula 9 where R₃ represents a protective group, many appropriate examples of which are known to one of skill in the art, as discussed below. The compound of formula 9 is then converted to sulfonamide of formula 10, the protective group is then cleaved to give a carboxylic acid of formula 4 and this compound is then coupled with an amine of formula 5 to give the compound of formula 1. It will be appreciated by one of skill in the art that Scheme 3 affords the possibility to carry out the sulfonylation reaction (the conversion of a compound of formula 9 to a compound of formula 10) on solid-phase by using a polymer-supported R₃ group.

Many protective groups R₃ are known to those of skill in the art of organic synthesis. For example, several suitable protective groups are enumerated in “Protective Groups in Organic Synthesis” [Greene, T. W. and Wuts, P. G. M., 2nd Edition, John Wiley & Sons, N.Y. 1991]. Preferred protective groups are those compatible with the reaction conditions used to prepare compounds of the invention. Examples of such protective groups are lower alkyl straight-chain or branched esters (e.g., methoxy (R₃═OCH₃), ethoxy (R₃═OCH₂CH₃), or tert-butoxy (R₃═OC(CH₃)3) esters), or the benzyl ester (R₃═OCH₂C₆H₅), or a resin commonly used in solid-phase synthesis (e.g., Wang resin or Rink resin), and these can be made by any conventional methods. For example, they may conveniently be made from the corresponding carboxylic acid of formula 2 by any esterification reaction, many of which are well known to one of ordinary skill in the art. For example, a compound of formula 9 in which R₃ represents methoxy can be prepared from a compound of formula 2 by treatment with an ethereal solution of diazomethane. The reaction is conveniently carried out in an inert solvent such as an ether (e.g., diethyl ether or tetrahydrofuran) or an alcohol (e.g., methanol), at a temperature of between about 0 degrees and about room temperature, preferably at about 0 degrees. In the case where R₃ represents the Wang resin, the compound of formula 9 is conveniently prepared by treating the resin with the compound of formula 2 in the presence of a coupling agent (such as diisopropylcarbodiimide) and in the presence of a catalytic amount of N,N-dimethylaminopyridine (DMAP) in an inert solvent such as N,N-dimethylformamide at about room temperature.

The sulfonylation reaction can be carried out by reacting the compound of formula 9 with a sulfonyl chloride of formula 3 in an inert solvent such as a halogenated hydrocarbon (such as methylene chloride) or an ether (such as tetrahydrofuran or dioxane) or an ester solvent such as ethyl acetate. The reaction is conveniently carried out in the presence of an organic base (such as triethylamine or diisopropylethylamine) or an inorganic base (such as sodium hydroxide or sodium carbonate). When an inorganic base is used, the reaction is conveniently carried out in the additional presence of water, and the co-solvent and protective group should be stable to the aqueous base. The reaction can be carried out at a temperature between about 0 degrees and about room temperature, preferably at around room temperature. Many sulfonyl chlorides of formula 3 are commercially available, or can be synthesized according to many different processes as discussed above.

For the removal of the protective group from a compound of formula 10 to give the carboxylic acid of formula 4, any conventional means can be used. For example, in the case where R₃ represents an unbranched lower alkoxy group (e.g., methoxy), the reaction may be carried out by treating the compound of formula 10 with an alkali methyl hydroxide, such as potassium hydroxide, sodium hydroxide or lithium hydroxide, preferably lithium hydroxide, in an appropriate solvent, such as a mixture of tetrahydrofuran, methanol and water. The reaction is conveniently carried out at a temperature between about 0 degrees and about room temperature, preferably at about room temperature. In the case where R₃ represents Wang resin or Rink resin, the cleavage can be effected using trifluoroacetic acid in dichloromethane at about room temperature.

The coupling of a carboxylic acid of formula 4 with an amine of formula 5 to give the compound of the invention of formula 1 according to Scheme 3, can be achieved as mentioned above, using methods well known to one of ordinary skill in the art. For example, the transformation can be carried out by reaction of carboxylic acids of formula 4 or of appropriate derivatives thereof such as activated esters, with amines of formula 5 or their corresponding acid addition salts (e.g., the hydrochloride salts) in the presence, if necessary, of a coupling agent, many examples of which are well known per se in peptide chemistry. The reaction is conveniently carried out by treating the carboxylic acid of formula 4 with the hydrochloride of the amine of formula 5 in the presence of an appropriate base, such as diisopropylethylamine, a coupling agent such as O-(benzotriazol-1-yl)-1,1,3,3-tetramethyluronium hexafluorophosphate, and in the optional additional presence of a substance that increases the rate of the reaction, such as 1-hydroxybenzotriazole or 1-hydroxy-7-azabenzotriazole, in an inert solvent, such as a chlorinated hydrocarbon (e.g., dichloromethane) or N,N-dimethylformamide or N-methylpyrrolidinone, at a temperature between about 0 degrees and about room temperature, preferably at about room temperature. Alternatively, the reaction can be carried out by converting the carboxylic acid of formula 4 to an activated ester derivative, such as the N-hydroxysuccinimide ester, and subsequently reacting this with the amine of formula 5 or a corresponding acid addition salt. This reaction sequence can be carried out by reacting the carboxylic acid of formula 4 with N-hydroxysuccinimide in the presence of a coupling agent such as N,N′-dicyclohexylcarbodiimide in an inert solvent such as tetrahydrofuran at a temperature between about 0 degrees and about room temperature. The resulting N-hydroxysuccinimide ester is then treated with the amine of formula S or a corresponding acid addition salt, in the presence of a base, such as organic base (e.g., triethylamine or diisopropylethylamine or the like) in a suitable inert solvent such as N,N-dimethylformamide at around room temperature.

### Sources of Racemic or Optically Active Nipecotic Acid of Formula 2

Racemic nipecotic acid is commercially from suppliers such as Aldrich Chemical Company, Inc., Milwaukee, Wisc.; TCI America, Portland, Oreg.; and Lancaster Synthesis Ltd., Lancashire, UK. The optically active nipecotic acids are also commercially available. For example, both (R)-(−)-nipecotic acid and (S)-(+)-nipecotic acid are available from the following suppliers:
Aldrich Chemical Company, Inc., Milwaukee, Wisc.
Digital Specialty Chemicals, Dublin, N.H.
TCI Japan, Tokyo, Japan
Yamakawa Chemical Industry Co., Ltd., Tokyo, Japan.

In addition, the individual enantiomers of nipecotic acid can be prepared by chiral chromatography (see J. S. Valsborg and C. Foged, J. Labelled Compd. Radiopharm. 1997, 39, 401) or by resolution. The following publications describe methods for the preparation by resolution of (R)-(−)-nipecotic acid and (S)-(+)-nipecotic acid or their acid addition salts:
M. Akkerman et al. Recueil Trav. Chim. Pays-Bas 1951, 70, 899
P. Magnus and L. S. Thurston J. Org. Chem. 1991, 56, 1166
X. Zheng et al. Chirality 1995, 7, 90
S. Schleich and G. Helmchen, Eur. J. Org. Chem. 1999, 2515
Chung, Y. J. et al. J. Am. Chem. Soc. 2000, 122, 3995
S. H. Gellman and B. R. Huck, U.S. Pat. No. 6,710,186
E. D. Moher et al, WO 2002068391
K. A. Ismail and S. C. Bergmaier, Eur. J. Med Chem. 2002, 37, 469

### Sources of Sulfonyl Chlorides of Formula 3

Sulfonyl chlorides of formula 3 can be purchased or they can be prepared using one of a large variety of different synthetic procedures well known in the field of organic synthesis, as outlined below. The synthetic approaches to sulfonyl chlorides are often complementary and offer access to sulfonyl chlorides with many different substitution patterns in the aryl ring system.

More than 100 sulfonyl chlorides of formula 3 are commercially available from suppliers such as Aldrich Chemical Company, Inc. (Milwaukee, Wisc.), Lancaster Synthesis Ltd. (Lancashire, UK), TCI America (Portland, Oreg.), and Maybridge plc (Tintagel, Cornwall, UK). For the purposes of illustration, a number of commercially available sulfonyl chlorides are shown in the table below. Many other examples can be found by consulting the Available Chemicals Directory (MDL Information Systems, San Leandro, Calif.) or SciFinder (Chemical Abstracts Service, Columbus, Ohio).

| Name | Supplier |
|---|---|
| 1-Naphthalene-sulfonyl chloride | TCI America, Portland, OR |
| 2,4-Difluoro-benzene-sulfonyl chloride | Aldrich Chemical Company, |
| | Inc., Milwaukee, WI |
| 2,5-Dichloro-benzene-sulfonyl chloride | Aldrich Chemical Company, |
| | Inc., Milwaukee, WI |
| 2-Chloro-6-methylbenzene-sulfonyl | Lancaster Synthesis Ltd., |
| chloride | Lancashire, UK |
| 2-Chloro-benzene-sulfonyl chloride | Aldrich Chemical Company, |
| | Inc., Milwaukee, WI |
| 2-Mesitylene-sulfonyl chloride | Lancaster Synthesis Ltd., |
| | Lancashire, UK |
| 3-Chloro-2-methylbenzene-sulfonyl | Maybridge plc, Tintagel, |
| chloride | Cornwall, UK |
| 3-Nitro-benzene-sulfonyl chloride | Aldrich Chemical Company, |
| | Inc., Milwaukee, WI |
| 3-Pyridinesulfonyl chloride | Combi-Blocks, LLC, |
| hydrochloride | San Diego, CA |
| 4-Methoxy-2,3,6-trimethyl-benzene- | Lancaster Synthesis Ltd., |
| sulfonyl chloride | Lancashire, UK |
| 8-Quinoline-sulfonyl chloride | Maybridge plc, Tintagel, |
| | Cornwall, UK |
| O-Toluene-sulfonyl chloride | TCI America, Portland, OR |

Sulfonyl chlorides of formula 3 can also be made by reactions that are well known in the field of organic synthesis, such as those outlined below.

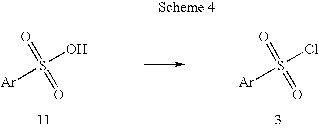

For example, sulfonyl chlorides of formula 3 can be made from a sulfonic acid of formula 11 as shown in Scheme 4. The chlorination of an arylsulfonic acid, or a salt thereof, of formula 11 can be accomplished conveniently by treating it with a chlorinating agent such as thionyl chloride or phosphorus oxychloride or phosphorus pentachloride, in the optional additional presence of a catalytic amount of N,N-dimethylformamide, at a temperature between about 0 degrees and about 80 degrees depending on the reactivity of the chlorinating agent. Many examples of this reaction are known in the literature, such as those listed in the following table

| Isoquinoline-5-sulfonyl chloride | A. Morikawa et al. J. Med. |
|---|---|
| | Chem. 1989, 32, 42 |
| 2-Ethoxycarbonyl-benzenesulfonyl | X. Baucherel et al. |
| chloride | WO 2002100810 |
| 4-n-Butoxybenzenesulfonyl chloride | V. P. Sandanayaka et al. |
| | U.S. Pat. No. 2002/0099035 |
| Benzothiazole-6-sulfonyl chloride | S. A. Kunda et al. |
| | U.S. Pat. No. 6,140,505 |
| 5-Dimethylamino-2-methyl- | C. Wu J. Org. Chem. |
| benzenesulfonyl chloride | 1998, 63, 2348 |

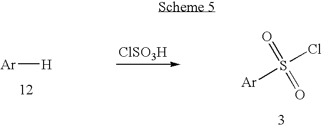

Sulfonyl chlorides of formula 3 can be made by electrophilic aromatic substitution of an aromatic compound of formula 12 as shown in Scheme 5. As is known to one of average skill in the art, this process is suitable for the preparation of arylsulfonyl chlorides with particular substitution patterns, such as for example where there is an ortho/para directing substituent in a benzene ring ortho or para to the site of introduction of the sulfonyl group. The reaction is conveniently carried out by treating the aromatic compound of formula 12 with chlorosulfonic acid in the absence of solvent and then heating the mixture at a temperature between about 70 degrees and about 100 degrees. Many examples of this reaction are known in the literature, such as those listed in the following table

| | |
|---|---|
| 5-Acetyl-3-thiophenesulfonyl | A. Arduini et al. Tetrahedron |
| chloride | Lett. 2003, 44, 5755 |
| 3-Bromo-5-isobutyl-thiophene-2- | V. Derdau et al. J. Org. Chem. |
| sulfonyl chloride | 2003, 68, 5168 |
| 2-Chloro-4-ethyl-thiazole-5- | R. Wischnat et al. WO 03002546 |
| sulfonyl chloride | |
| 4-(1,3-Dihydro-1,3-dioxo-2H- | L. M. Lima et al. Bioorg. Med. |
| isoindol-2-yl)- | Chem. 2002, 10, 3067 |
| benzenesulfonyl chloride | |
| 2,3-Dihydro-6-methoxy-1H-Indene- | M. A. Aboud-Gharbia |
| 5-sulfonyl chloride | U.S. Pat. No. 4,857,644 |
| 5-(1,1-Dimethylethyl)-2-methyl- | Y. Christidis |
| benzenesulfonyl chloride | U.S. Pat. No. 4,948,827 |
| 4-Fluoro-2-methyl-benzenesulfonyl | M. Pal et al. J. Med. Chem. |
| chloride | 2003, 46, 3975 |
| 1-Methyl-1H-pyrazole-4-sulfonyl | P. J. Dollings et al. |
| chloride | U.S. Pat. No. 6,103,708 |
| [4-(Chlorosulfonyl)phenyl]- | B. P. Clark |
| carbamic acid, methyl ester | U.S. Pat. No. 6,482,824 |
| 1,2,3,4-Tetrahydro-6-methyl-2,4- | V. V. Makarov et al. |
| dioxo-5-pyrimidinesulfonyl chloride | RU 2,204,555 (Chemical |
| | Abstracts CAN 140: 93843) |

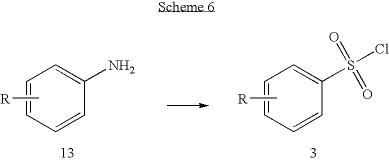

Sulfonyl chlorides of formula 3 can also be made from anilines of formula 13 by a diazotization/sulfonylation reaction sequence as shown in Scheme 6. The diazotization reaction is conveniently carried out by treating the aniline of formula 13 or an acid addition salt thereof (such as the hydrochloride salt) in aqueous solution in the presence of a mineral acid such as hydrochloric acid or sulfuric acid with an alkali metal nitrite salt such as sodium nitrite at a temperature less than 10 degrees, preferably around 0 degrees. The diazonium salt obtained in this way can be converted directly to the sulfonyl chloride using a variety of reagents and conditions which are known in the field of organic synthesis. Examples of suitable reagents include sulfur dioxide and copper(I) chloride or copper(II) chloride in acetic acid/water, or thionyl chloride and copper(I) chloride or copper(II) chloride in water, according to the procedure of P. J. Hogan (U.S. Pat. No. 6,531,605). For example, the sulfonylation reaction can be carried out by adding the solution of the diazonium salt, prepared as described above, to a mixture of sulfur dioxide and copper(II) chloride in a suitable inert solvent, such as glacial acetic acid, at a temperature around 0 degrees. Many examples of this reaction are known in the literature, such as those listed in the following table

| | |
|---|---|
| 4-Methyl-benzenesulfonyl chloride | N. Ikemoto et al. Tetrahedron |
| | 2003, 59, 1317 |
| 3,4,5-Trimethoxy-benzenesulfonyl | C. Binisti et al. Eur. J. Med. |
| chloride | Chem. 2001, 36, 809 |
| 2-Fluoro-6-trifluoromethyl- | M. A. Gonzalez and E. W. |
| benzenesulfonyl chloride | Otterbacher U.S. Pat. No. |
| | 6,433,169 |
| 2-Methoxy-pyridine-5-sulfonyl | S. L. Gwaltney et al. Bioorg. |
| chloride | Med. Chem. Lett. 2001, 11, 871 |
| 3-Nitro-benzenesulfonyl chloride | M. Meier and R. Wagner |
| | U.S. Pat. No. 5,436,370 |
| 4-Benzyloxy-2-nitro-benzenesulfonyl | R. J. Cherney et al. J. Med. |
| chloride | Chem. 2003, 46, 1811 |
| 4-Acetyl-benzenesulfonyl chloride | A. S. Wagman et al. J. Org. |
| | Chem. 2000, 65, 9103 |

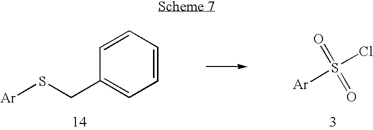

Sulfonyl chlorides of formula 3 can also be made from an aryl benzyl sulfide of formula 14 by an oxidative chlorination reaction as shown in Scheme 7. The reaction is conveniently carried out by bubbling chlorine gas into a solution or suspension of the aryl benzyl sulfide of formula 14 in a suitable solvent such as a mixture of acetic acid and water at a temperature around room temperature.

| | |
|---|---|
| 4-(Chlorosulfonyl)-3-nitro-benzoic | S. P. Andrews et al.. Org. |
| acid, methyl ester | Chem. 2003, 68, 5525 |
| 4,7-Dichloro-quinoline-6-sulfonyl | R. H. Baker et al. J. Am. |
| chloride | Chem. Soc. 1946, 68, 2636 |
| 1,3-Dioxo-2,3-dihydro-2-methyl-1H- | J. V. Hay et al. |
| isoindol4-4-sulfonyl chloride | U.S. Pat. No. 4,521,241 |
| 2,3-Dihydro-1-oxo-1H-indene-5- | J. J. Howbert and T. A. |
| sulfonyl chloride | Crowell Synthetic |
| | Commun. 1990, 20, 3193 |
| 5-(2-Chlorosulfonyl-phenyl)-3- | W. J. Barry and I. L. Finar J. |
| methyl-1-phenyl-1H-pyrazole-4- | Chem. Soc. 1954, 138 |
| carboxylic acid ethyl ester | |
| 3-Methyl-4-nitro-benzenesulfonyl | J. C. Baum et al. Can. |
| chloride | J. Chem. 1990, 68, 1450 |

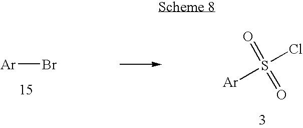

Sulfonyl chlorides of formula 3 can also be made as shown in Scheme 8 from an aryl bromide of formula 15 by metal-halogen exchange, followed by reaction of the organometallic intermediate with sulfur dioxide to give an arylsulfonate salt, followed by reaction with sulfuryl chloride to give the arylsulfonyl chloride. The reaction can be carried out by treating the aryl bromide with an organometallic reagent such as n-butyl lithium or preferably sec-butyl lithium, in the optional additional presence of tetramethylethylenediamine (TMEDA) in a suitable inert solvent such as tetrahydrofuran (THF) or diethyl ether at low temperature (for example, around −78 degrees) to give the aryllithium intermediate. This can then be reacted, without isolation, with a mixture of sulfur dioxide and a solvent such as diethyl ether, again at low temperature, such as for example between about -78 degrees and about −60 degrees. The resulting arylsulfonate salt can then be converted to the arylsulfonyl chloride, again without isolation of the intermediate, by treatment with sulfuryl chloride at a temperature around 0 degrees. Many examples of this reaction are known in the literature, such as those listed in the following table

| | |
|---|---|
| 2-Benzyloxy-5-methyl-benzenesulfonyl | G. Papageorgiou et al. |
| chloride | Tetrahedron 1999, 55, 237 |
| [2,2′]Bithiophenyl-5-sulfonyl chloride | M. F. Chan et al. Bioorg. |
| | Med. Chem. 1998, 6, 2301 |
| 2′-Methoxy-biphenyl-4-sulfonyl | W. R. Ewing et al. J. Med. |
| chloride | Chem. 1999, 42, 3557 |
| 4-(2-Phenyl-2H-tetrazol-5-yl)- | Y. Tamura et al. J. Med. |
| benzenesulfonyl chloride | Chem. 1998, 41, 640 |
| 3-(2-p-Tolyl-vinyl)-thiophene-2- | B. Raju et al. Bioorg. Med |
| sulfonyl chloride | Chem. Lett. 1997, 7, 939 |
| 3-Trifluoromethyl-benzenesulfonyl | T. Hamada and O. Yonemitsu |
| chloride | Synthesis 1986, 852 |

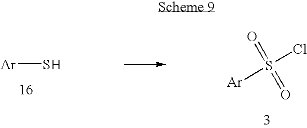

Sulfonyl chlorides of formula 3 can be made from an aryl thiol of formula 16 by oxidation using chlorine as shown in Scheme 9. For example, the reaction can be carried out by treating the aryl thiol of formula 16 with a solution of chlorine in an inert solvent such as glacial acetic acid at a temperature around 0 degrees. For example, 4-(1H-tetrazol-1-yl)phenyl]sulfonyl chloride could be prepared using this procedure from the thiophenol 4-(1H-tetrazol-1-yl)-benzenethiol which is known (W. V. Curran et al. U.S. Pat. No. 3,932,440). Several examples of this reaction are known in the literature, such as those listed in the following table

| | |
|---|---|
| 2-Benzothiazolesulfonyl chloride | E. Vedejs et al. J. Org. Chem. |
| | 2000, 65, 2309 |
| 5-(Chlorosulfonyl)-1-methyl-1H- | F. Suzuki et al. JP 06056792 |
| pyrazole-4-carboxylic acid, | Chemical Abstracts CAN 122: 31573 |
| ethyl ester | |
| 5-Amino-1H-1,2,4-Triazole-3- | R. B. Shankar |
| sulfonyl chloride | U.S. Pat. No. 4,937,350 |
| 2-Methyl-benzenesulfonyl chloride | G. E. Lepone |
| | U.S. Pat. No. 4,454,135 |

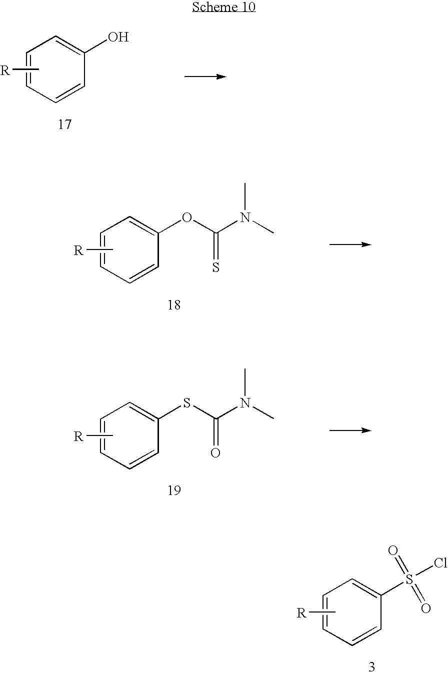

Sulfonyl chlorides of formula 3 can be made from a phenol of formula 17 through a sequence of reactions outlined in Scheme 10. The phenol of formula 17 can be converted to the O-aryl-N,N′-dialkylthiocarbamate of formula 18 by reaction with an N,N′-dialkylthiocarbamoyl chloride in an inert solvent in the presence of a base. The resulting O-aryl-N,N′-dialkylthiocarbamate of formula 18 can be rearranged to the S-aryl-N,N′-dialkylthiocarbamate of formula 19 by heating neat at high temperature such as at around 250 degrees. The S-aryl-N,N′-dialkylthiocarbamate of formula 19 can then be converted to the sulfonyl chloride of formula 3 by oxidation using chlorine in a suitable inert solvent such as a mixture of formic acid and water at a temperature around 0 degrees. An example of the use of this process for the preparation of sulfonyl chlorides can be seen in V. Percec et al. J. Org. Chem. 2001, 66, 2104.

### Sources of Amines of Formula 5

Amines of formula 5 can be purchased or they can be prepared using one of a large variety of different synthetic procedures well known in the field of organic synthesis, as outlined below.

Several thousand amines of formula 5 are commercially available from suppliers such as Aldrich Chemical Company, Inc. (Milwaukee, Wisc.), Lancaster Synthesis Ltd. (Lancashire, UK), TCI America (Portland, Oreg.), and Maybridge plc (Tintagel, Cornwall, UK). Other examples of amines are found in the Available Chemicals Directory (MDL Information Systems, San Leandro, Calif.) or SciFinder (Chemical Abstracts Service, Columbus, Ohio).

Amines of formula 5 can also be made by reactions that are well known in the field of organic synthesis, such as those outlined in “Comprehensive Organic Transformations: A Guide to Functional Group Preparations” [R. C. Larock, VCH Publishers, Inc., N.Y. 1989, pages 385-438] and in “Advanced Organic Chemistry” [J. March, 3^{rd} Edition, Wiley Interscience, NY, 1985].

Resin-bound amines of formula 5 in which R₂ represents a resin to which an amine can be attached can be prepared by reactions that are familiar to one of average skill in the art of solid-phase organic synthesis. For example, an amine of formula 5 where R₂ represent the FMPB resin can be prepared according to Scheme 11 by treating FMPB resin (20) with a primary amine of formula 21 in the presence of a reducing agent such as sodium triacetoxyborohydride in an inert solvent such as a halogenated hydrocarbon (such as 1,2-dichloroethane) at room temperature.

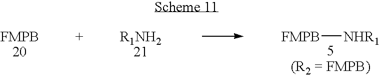

Some examples of amines that can be prepared by known methods are shown in the table below:

| | |
|---|---|
| Tetrahydro-N-methyl-3-Thiophenamine, | B. Loev J. Org. Chem. |
| 1,1-dioxide | 1961, 26, 4394 |
| Tetrahydro-3-thiophenamine, | Thomas P. Johnston et al. |
| 1,1-dioxide | J. Med. Chem. 1971, |
| | 14, 600 |
| 2-Cyclohex-1-enyl-ethylamine | R. S. Coleman and J. A. |
| | Shah Synthesis 1999, 1399 |
| N-[(4-Fluorophenyl)methyl]- | S. Casadio Bollettino Chimico |
| benzeneethanamine, hydrochloride | Farmaceutico 1978, V117, |
| | P83-9 Chemical Abstracts CAN |
| | 90: 16185 |
| 3-Isopropoxypropylamine | J. C. Little |
| | U.S. Pat. No. 3,372,195 |
| endo-Norbornylamine | R. F. Borch et al. J. Am. |
| | Chem. Soc. 1971, 93, 2897 |
| N-Cyclopropyl-N-(2-thienylmethyl)- | N. R. Easton DE 1,568,438 |
| amine | |
| Bis-(2-methoxy-ethyl)-amine | Monsanto Chm. Co. |
| | U.S. Pat. No. 2,876,243 |

In addition, a series of aminomethylpyrazoles can be prepared using the reductive amination procedure described by Borch et al (R. F. Borch et al. J. Am. Chem. Soc. 1971, 93, 2897), starting from pyrazole-carboxaldehydes that are commercially available, as shown in the table below:

| Amine | Aldehyde | Aldehyde Supplier |
|---|---|---|
| 1,3,5-Trimethyl-1H- | 1,3,5-Trimethyl-1H- | Maybridge plc, |
| pyrazole-4-methylamine | pyrazole-4- | Tintagel, |
| | carbaldehyde | Cornwall, UK |
| 1,5-Dimethyl-1H- | 1,5-Dimethyl-1H- | Fluorochem Ltd., |
| pyrazole-4-methylamine | pyrazole-4- | Old Glossop, |
| | carbaldehyde | Derbyshire, UK |
| 1,3-Dimethyl-1H- | 1,3-Dimethyl-1H- | Acros Organics |
| pyrazole-4-methylamine | pyrazole-4- | USA, Morris |
| | carbaldehyde | Plains, NJ |
| 5-Chloro-1,3-dimethyl- | 5-Chloro-1,3-dimethyl- | Key Organics |
| 1H-pyrazole-4- | 1H-pyrazole-4- | Limited/Bionet |
| methylamine | carbaldehyde | Research, |
| | | Camelford, UK |
| 4-Chloro-1-methyl-1H- | 4-Chloro-1-methyl-1H- | Butt Park Ltd., |
| pyrazole-3-methylamine | pyrazole-3-carbaldehyde | Bath, UK |
| 4-Bromo-1-methyl-1H- | 4-Bromo-1-methyl-1H- | Apollo Scientific |
| pyrazole-3-methylamine | pyrazole-3-carbaldehyde | Ltd., |
| | | Stockport, UK |
| 1-Methyl-1H-pyrazole-4- | 1-methyl-1H-pyrazole-4- | Fluorochem Ltd., |
| methylamine | carbaldehyde | Old Glossop, |
| | | Derbyshire, UK |
| 1-Ethyl-5-methyl-1H- | 1-Ethyl-5-methyl-1H- | Fluorochem Ltd., |
| pyrazole-4-methylamine | pyrazole-4-carbaldehyde | Old Glossop, |
| | | Derbyshire, UK |
| 1-Ethyl-3-methyl-1H- | 1-Ethyl-3-methyl-1H- | Fluorochem Ltd., |
| pyrazole-4-methylamine | pyrazole-4-carbaldehyde | Old Glossop, |
| | | Derbyshire, UK |
| 1-Ethyl-1H-pyrazole-4- | 1-Ethyl-1H-pyrazole-4- | Fluorochem Ltd., |
| methylamine | carbaldehyde | Old Glossop, |
| | | Derbyshire, UK |
| 1-Ethyl-1H-pyrazole- | 1-Ethyl-1H-pyrazole-2,5- | N. D. Zelinsky |
| 2,5-dimethyl-4- | dimethyl-4-carbaldehyde | Institute, |
| methylamine | | Moscow, Russia |
| 1,3-Dimethyl-1H- | 1,3-Dimethyl-1H- | Maybridge plc, |
| pyrazole-5-methylamine | pyrazole-5-carbaldehyde | Tintagel, |
| | | Cornwall, UK |
| 3-Methyl-1-propyl-1H- | 3-Methyl-1-propyl-1H- | Ost-West |
| pyrazole-4-methylamine | pyrazole-4-carbaldehyde | Handelsservice, |
| | | Zepernick, Germany |
| 4-Bromo-1-methyl-1H- | 4-Bromo-1-methyl-1H- | Maybridge plc, |
| pyrazole-5-methylamine | pyrazole-5-carbaldehyde | Tintagel, |
| | | Cornwall, UK |
| 5-Chloro-3-ethyl-1- | 5-Chloro-3-ethyl-1- | Oakwood Products, |
| methyl-1H-pyrazole-4- | methyl-1H-pyrazole-4- | Inc., West |
| methylamine | carboxaldehyde | Columbia, SC |

General Synthesis of Adamantanamines

Amines of formula 5 in which R₁ represents hydrogen and R₂ represents unsubstituted or substittued adamantane are either commercially available or can be made by methods that are well known to one of average skill in the art. Examples of commercially available adamantan-1-yl-amines are shown in the table below.

| Name | Supplier |
|---|---|
| 1-Adamantanamine | Aldrich Chemical Company, |
| | Inc., Milwaukee, WI |
| 2-Adamantanamine hydrochloride | Aldrich Chemical Company, |
| | Inc., Milwaukee, WI |
| 3,5,7-Trimethyl-1-adamantanamine | ChemDiv, Inc., |
| | San Diego, CA |
| 3,5-Bis(1-methylethyl)-1- | MicroChemistry Ltd., |
| adamantanamine hydrochloride | Moscow, Russia |
| 3-Amino-1-adamantanol | Aldrich Chemical Company, |
| | Inc., Milwaukee, WI |
| 3-Cyclohexyl-1-adamantanamine | MicroChemistry Ltd., Moscow, |
| hydrochloride | Russia |
| 3-Ethyl-1-adamantanamine | Apin Chemicals Ltd., |
| hydrochloride | Abingdon, UK |
| 3-Ethyl-5,7-dimethyl-1- | MicroChemistry Ltd., Moscow, |
| adamantanamine hydrochloride | Russia |
| 3-Ethyl-5-methyl-1-adamantanamine | MicroChemistry Ltd., Moscow, |
| hydrochloride | Russia |
| 3-Isopropyl-1-adamantanamine | Chembridge, San Diego, CA |
| 3-Methyl-1-adamantanamine | Ambinter, Paris, France |
| hydrochloride | |
| 3-n-Propyl-1-adamantanamine | ChemDiv, Inc., |
| | San Diego, CA |
| 3-Trifluoromethyl-1-adamantanamine | Interchim, Montlucon, |
| hydrochloride | France |
| 4-Amino-1-adamantanol | MicroChemistry Ltd., Moscow, |
| | Russia |
| 5-Amino-2-adamantanol | MicroChemistry Ltd., Moscow, |
| | Russia |
| 5-Amino-3,7-dimethyl-adamantan-1-ol | MicroChemistry Ltd., Moscow, |
| | Russia |
| (5-Amino-3-methyl-adamantan-1-yl)- | ChemDiv, Inc., |
| methanol | San Diego, CA |
| Memantine hydrochloride | Sigma, St. Louis, MOI |

Amines of formula 5 in which R₁ represents hydrogen and R₂ represents unsubstituted or substituted adamantane which are not commercially available can be made using a number of different reactions known in the literature. For example, 2-adamantanamine derivatives can be prepared from the corresponding adamantan-2-ones by conversion of the ketone to the oxime followed by reduction to the amine. Such reactions can be carried out using the procedures described in K. Banert et al. Chem. Ber. 1986, 119, 3826-3841. 2-Adamantanamines can also be prepared from 4-alkyl-4-protoadamantanols by a Ritter reaction with acetonitrile in the presence of sulfuric acid to give the acetamide which is then hydrolyzed to give the 2-adamantanamine, as described in D. Lenoir et al. J. Org. Chem. 1971, 36, 1821-1826.

Adamantanamines can be prepared from the corresponding 1-adamantane-carboxamides using a Hoffmann rearrangement or similar reaction. A variety of conditions for effecting this reaction are known in the art, and there have been a number of publications disclosing the application of this reaction for the preparation of 1-adamantanamines. Among these are the hypervalent iodine-mediated Hoffmann rearrangement described in R. M. Moriarty et al. Synth. Commun. 1988, 18, 1179 and G. Loudon et al. J. Org Chem. 1984, 49, 4272-4276, and the hypochlorite-mediated reaction reported in G. L. Anderson et al. Synth. Commun. 1988, 18, 1967. 1-Adamantanamines can also be prepared using the Ritter reaction starting from the corresponding 1-adamantanol and treating with chloro-acetonitrile under acidic conditions, followed by hydrolysis of the amide. The preparation of 1-adamantanamine using such a process has been described by A. Jirgensons et al. in Synthesis 2000, 1709-1712. Alternatively, 1-adamantanamines can be prepared from the corresponding 1-bromo-adamantanes using either Ritter-like conditions followed by hydrolysis (see K. Gerzon et al. J. Med. Chem. 1963, 6, 760-763 or O. Cervinka et al. Collect. Czech Chem. Commun. 1974, 39, 1592-1588), or by reaction of the 1-bromo-adamantanes with acetamide followed by hydrolysis (see K. Gerzon et al. J. Med. Chem. 1967, 10, 603-606). The 1-bromo-adamantanes are readily available by bromination of the hydroxy-adamantanes using bromine/triphenylphosphine or from the adamantane using bromine (see J. G. Henkel et al. J. Med Chem. 1982, 25, 51-56). 1-Adamantanamines can also be prepared from the corresponding 1-adamantanols by displacement of the hydroxy group by azide under acidic conditions, followed by reduction of the azide (see T. Sasaki et al. J. Org. Chem. 1977, 42, 3741-3743).

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as an “effective amount”. For example, the dose of a compound of the present invention is typically in the range of about 10 to about 1000 mg per day.

The invention will now be further described in the Examples below, which are intended as an illustration only and do not limit the scope of the invention.

### EXAMPLES

### Part I: Preferred Intermediates

The following reagents were obtained from the vendors listed in the table, unless otherwise indicated in the experimental descriptions.

| Starting Material | Supplier |
|---|---|
| 4-Acetamido-benzenesulfonyl chloride | Aldrich Chemical Company, |
| | Inc., Milwaukee, WI |
| 1-Adamantanamine | Aldrich Chemical Company, |
| | Inc., Milwaukee, WI |
| 1-Aminoindan | Aldrich Chemical Company, |
| | Inc., Milwaukee, WI |
| 2-Amino-1-methoxybutane | TCI America, Portland, OR |
| Benzenesulfonyl chloride | Aldrich Chemical Company, |
| | Inc., Milwaukee, WI |
| Benzylamine | Aldrich Chemical Company, |
| | Inc., Milwaukee, WI |
| 4-Bibenzenesulfonyl chloride | Fluka Chemical Corp., |
| | Milwaukee, WI |
| 4-n-Butyl-benzenesulfonyl chloride | Maybridge plc, Tintagel, |
| | Cornwall, UK |
| 4-tert-Butylcyclohexylamine | TCI America, Portland, OR |
| 2-Chlorobenzenesulfonyl chloride | Aldrich Chemical Company, |
| | Inc., Milwaukee, WI |
| 2-Chlorobenzenesulfonyl chloride | Aldrich Chemical Company, |
| | Inc., Milwaukee, WI |
| 3-Chlorobenzenesulfonyl chloride | Lancaster Synthesis Ltd., |
| | Lancashire, UK |
| 4-Chlorobenzenesulfonyl chloride | Aldrich Chemical Company, |
| | Inc., Milwaukee, WI |
| 2-Chloro-benzylamine | Aldrich Chemical Company, |
| | Inc., Milwaukee, WI |
| 3-Chloro-4-fluoro-benzenesulfonyl | Alfa Aesar, Ward Hill, MA |
| chloride | |
| 3-Chloro-2-methyl-benzenesulfonyl | Aldrich Chemical Company, |
| chloride | Inc., Milwaukee, WI |
| 2-(3-Chlorophenyl)ethylamine | Aldrich Chemical Company, |
| | Inc., Milwaukee, WI |
| Cyclohexylamine | Eastman Kodak, Rochester, NY |
| Cyclopentylamine | Lancaster Synthesis Ltd., |
| | Lancashire, UK |
| Decahydroisoquinoline | Aldrich Chemical Company, |
| | Inc., Milwaukee, WI |
| trans-Decahydroisoquinoline | TCI America, Portland, OR |
| Decahydroquinoline | Aldrich Chemical Company, |
| | Inc., Milwaukee, WI |
| Decahydroquinoline | Aldrich Chemical Company, |
| | Inc., Milwaukee, WI |
| 2,4-Dichlorobenzenesulfonyl chloride | Aldrich Chemical Company, |
| | Inc., Milwaukee, WI |
| 2,4-Dichlorobenzenesulfonyl chloride | Aldrich Chemical Company, |
| | Inc., Milwaukee, WI |
| 1-(3-Dimethylaminopropyl)-3- | Advanced ChemTech, |
| ethylcarbodiimide hydrochloride | Louisville, KY |
| N,N-Dimethylaminopyridine | Aldrich Chemical Company, |
| | Inc., Milwaukee, WI |
| 4-Fluoro-benzenesulfonyl chloride | Aldrich Chemical Company, |
| | Inc., Milwaukee, WI |
| 1-(4-Fluorophenyl)ethylamine | Aldrich Chemical Company, |
| | Inc., Milwaukee, WI |
| 2-(2-Fluorophenyl)ethylamine | Aldrich Chemical Company, |
| | Inc., Milwaukee, WI |
| 2-(4-Fluorophenyl)ethylamine | Aldrich Chemical Company, |
| | Inc., Milwaukee, WI |
| Hexamethyleneimine | Aldrich Chemical Company, |
| | Inc., Milwaukee, WI |
| Hexamethyleneimine | Aldrich Chemical Company, |
| | Inc., Milwaukee, WI |
| 1-Hydroxybenzotriazole hydrate | Acros Organics USA, |
| | Morris Plains, NJ |
| 4-Hydroxypiperidine | Aldrich Chemical Company, |
| | Inc., Milwaukee, WI |
| 4-Hydroxy-piperidine | Fluka Chemical Corp., |
| | Milwaukee, WI |
| Isoamylamine | Aldrich Chemical Company, |
| | Inc., Milwaukee, WI |
| Isoamylamine | Aldrich Chemical Company, |
| | Inc., Milwaukee, WI |
| Isobutylamine | Aldrich Chemical Company, |
| | Inc., Milwaukee, WI |
| Isopropylamine | Aldrich Chemical Company, |
| | Inc., Milwaukee, WI |
| 4-Isopropyl-benzenesulfonyl chloride | Aldrich Chemical Company, |
| | Inc., Milwaukee, WI |
| Lithium hydroxide monohydrate | Aldrich Chemical Company, |
| | Inc., Milwaukee, WI |
| 4-Methoxy-benzenesulfonyl chloride | Aldrich Chemical Company, |
| | Inc., Milwaukee, WI |
| 2-Methoxy-benzylamine | Aldrich Chemical Company, |
| | Inc., Milwaukee, WI |
| 2-(Methoxycarbony)-benzenesulfonyl | Alfa Aesar, Ward Hill, MA |
| chloride | |
| 2-(2-Methoxyphenyl)ethylamine | TCI America, Portland, OR |
| 3-Methoxypropylamine | Lancaster Synthesis Ltd., |
| | Lancashire, UK |
| Methylamine | Aldrich Chemical Company, |
| | Inc., Milwaukee, WI |
| 2-Methyl-benzylamine | Aldrich Chemical Company, |
| | Inc., Milwaukee, WI |
| dl-alpha-Methylbenzylamine | Aldrich Chemical Company, |
| | Inc., Milwaukee, WI |
| 4-Methylpiperidine | Aldrich Chemical Company, |
| | Inc., Milwaukee, WI |
| 4-Methyl-piperidine | Aldrich Chemical Company, |
| | Inc., Milwaukee, WI |
| Morpholine | Aldrich Chemical Company, |
| | Inc., Milwaukee, WI |
| 2-(4-Morpholino)-ethylamine | TCI America, Portland, OR |
| 1-Naphthalenemethylamine | Aldrich Chemical Company, |
| | Inc., Milwaukee, WI |
| 2-Naphthylsulfonyl chloride | Aldrich Chemical Company, |
| | Inc., Milwaukee, WI |
| Nipecotic acid ethyl ester | Aldrich Chemical Company, |
| | Inc., Milwaukee, WI |
| Phenethylamine | Aldrich Chemical Company, |
| | Inc., Milwaukee, WI |
| 2-Phenyl-propylamine | Aldrich Chemical Company, |
| | Inc., Milwaukee, WI |
| 3-Phenyl-propylamine | Aldrich Chemical Company, |
| | Inc., Milwaukee, WI |
| 8-Quinolinesulfonyl chloride | Lancaster Synthesis Ltd., |
| | Lancashire, UK |
| 1,2,3,4-Tetrahydro-1-naphthylamine | Aldrich Chemical Company, |
| | Inc., Milwaukee, WI |
| Thiophene-2-sulfonyl chloride | Aldrich Chemical Company, |
| | Inc., Milwaukee, WI |
| Thiophene-2-sulfonyl chloride | Aldrich Chemical Company, |
| | Inc., Milwaukee, WI |
| Triethylamine | Aldrich Chemical Company, |
| | Inc., Milwaukee, WI |
| 2-(Trifluoromethyl)-benzylamine | Aldrich Chemical Company, |
| | Inc., Milwaukee, WI |

### Intermediate A1: (3R)-1-(2-Chloro-benzenesulfonyl)-piperidine-3-carboxylic acid

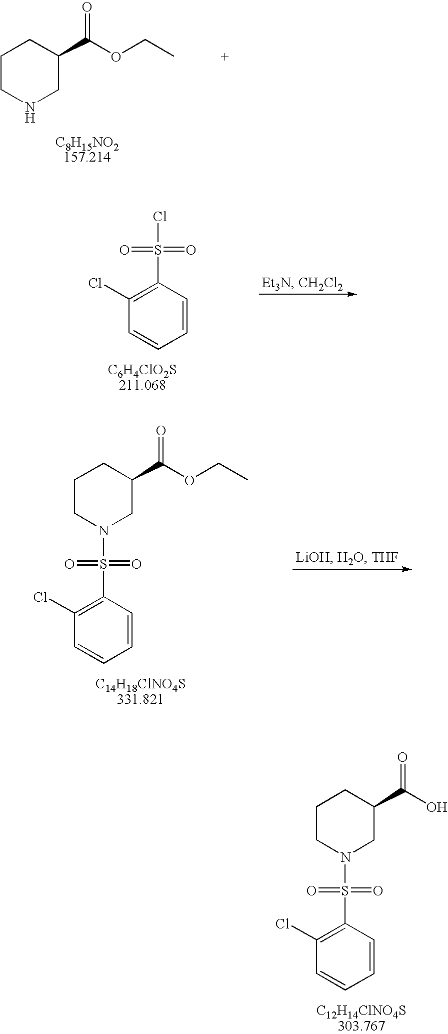

### Step 1: (3R)-1-(2-Chloro-benzenesulfonyl)-piperidine-3-carboxylic acid ethyl ester

Chlorobenzenesulfonyl chloride (0.25 mL, 1.8 mmol) was added to a solution of (R)-(+)-nipecotic acid ethyl ester (available from Aldrich Chemical Company; Inc., Milwaukee, Wisc.; 250 mg, 1.6 mmol) and triethylamine (0.5 mL, 3.6 mmol) in dichloromethane (5 mL) under argon. An additional portion of dichloromethane (10 mL) was added and the solution was stirred for five days at room temperature. The reaction mixture was washed with water and the water layer was back-extracted with dichloromethane. The combined organic layers were washed with 80% saturated brine, dried (magnesium sulfate), filtered and evaporated to give (3R)-1-(2-chloro-benzenesulfonyl)-piperidine-3-carboxylic acid ethyl ester (561 mg) as a colorless viscous oil, which was used directly in the next step. NMR indicated the presence of the desired product along with a small amount of dichloromethane.

### Step 2: (3R)-1-(2-Chloro-benzenesulfonyl)-piperidine-3-carboxylic acid

1 M Aqueous lithium hydroxide solution (3.5 mL) was added to a solution of (3R)-1-(2-chloro-benzenesulfonyl)-piperidine-3-carboxylic acid ethyl ester (from Step 1; 560 mg) in tetrahydrofuran (10 mL). The reaction mixture was stirred overnight at room temperature, the solvent was evaporated, the residue was diluted with water and the solution was acidified to pH 1. The solution was extracted three times with ethyl acetate, and the combined organic layers were washed with 80% saturated brine, dried (magnesium sulfate), filtered and evaporated to give (3R)-1-(2-chloro-benzenesulfonyl)-piperidine-3-carboxylic acid (450 mg, 92%) as a colorless semisolid.

### Intermediate A2: (3S)-1-(2-Chloro-benzenesulfonyl)-piperidine-3-carboxylic acid

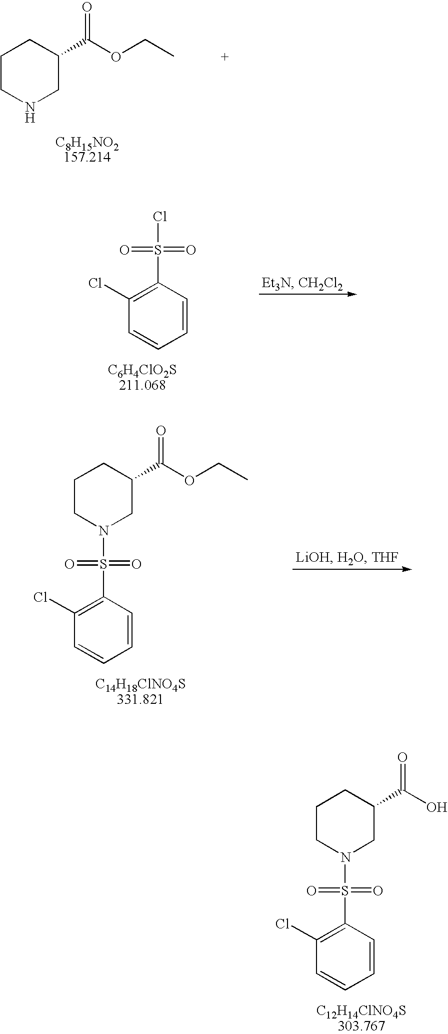

(3S)-1-(2-Chloro-benzenesulfonyl)-piperidine-3-carboxylic acid was prepared from 2-chlorobenzenesulfonyl chloride and (S)-(+)-nipecotic acid ethyl ester (available from Aldrich Chemical Company, Inc., Milwaukee, Wisc.; 166 mg, 1.1 mmol) using the procedure described for the preparation of Intermediate A1.

### Intermediate A3: (rac)-1-(2-Chloro-benzenesulfonyl)-piperidine-3-carboxylic acid

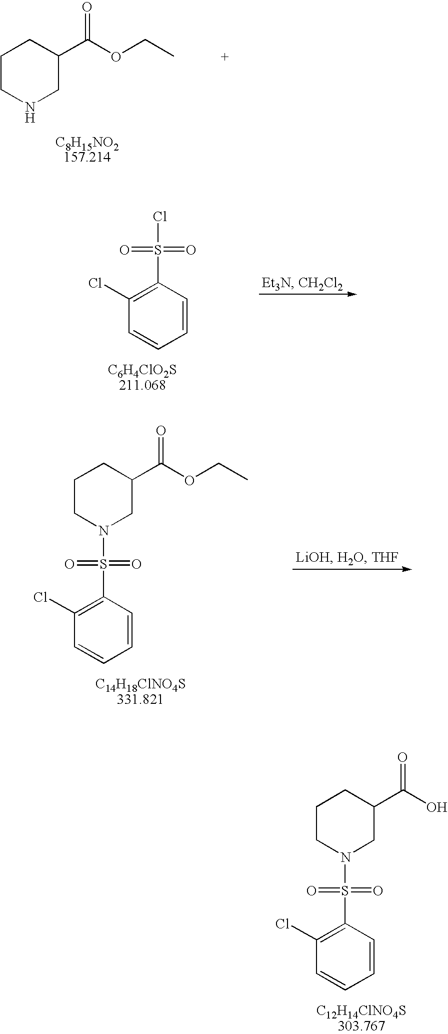

(rac)-1-(2-Chloro-benzenesulfonyl)-piperidine-3-carboxylic acid was prepared from 2-chlorobenzenesulfonyl chloride and (rac)-nipecotic acid ethyl ester using the procedure described for the preparation of Intermediate A1.

### Intermediate A4: (3R)-1-(4-Chloro-benzenesulfonyl)-piperidine-3-carboxylic acid

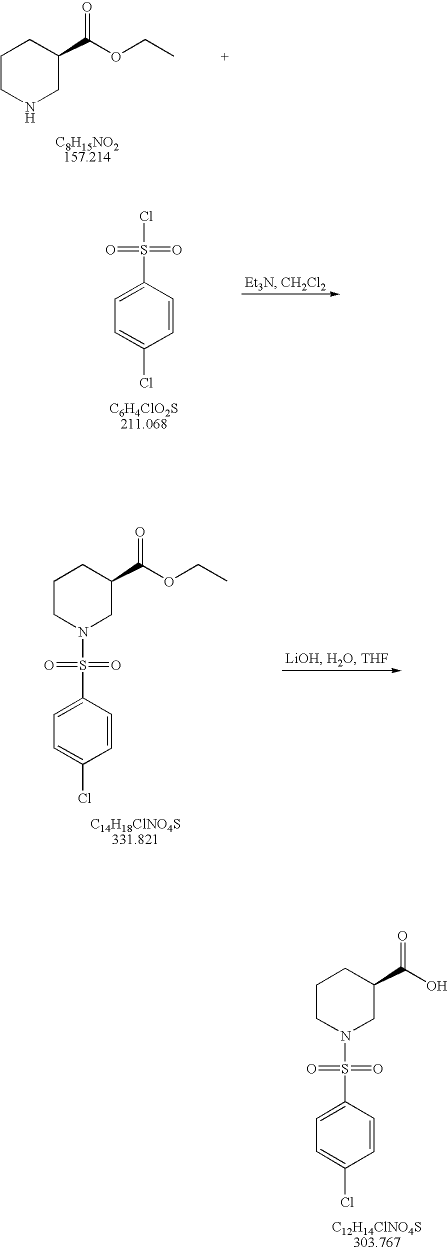

(3R)-1-(4-Chloro-benzenesulfonyl)-piperidine-3-carboxylic acid was prepared from 4-chlorobenzenesulfonyl chloride and (R)-(+)-nipecotic acid ethyl ester (available from Aldrich Chemical Company, Inc., Milwaukee, Wisc.) using the procedure described for the preparation of Intermediate A1.

### Intermediate A5: (3S)-1-(2,4-Dichloro-benzenesulfonyl)-piperidine-3-carboxylic acid

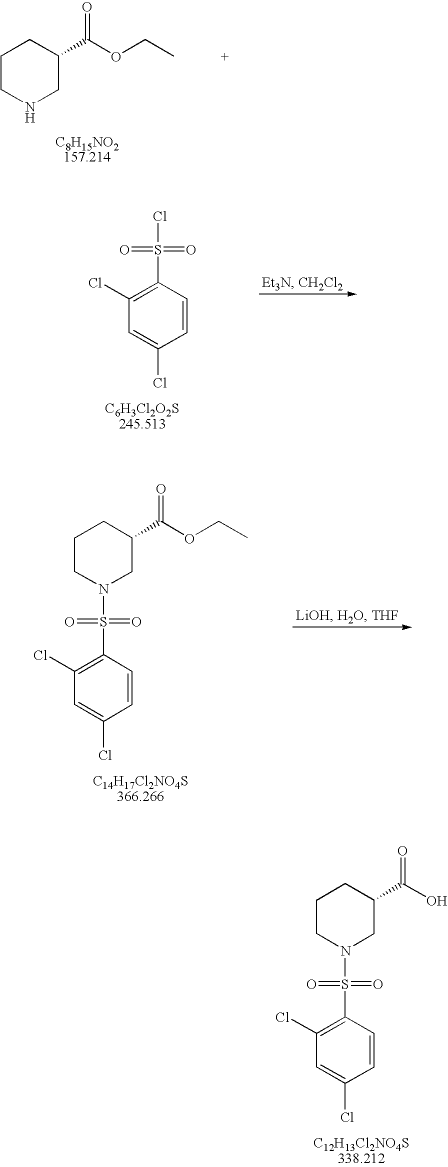

(3S)-1-(2,4-Dichloro-benzenesulfonyl)-piperidine-3-carboxylic acid was prepared from 2,4-dichlorobenzenesulfonyl chloride and (S)-(−)-nipecotic acid ethyl ester (available from Aldrich Chemical Company, Inc., Milwaukee, Wisc.) using the procedure described for the preparation of Intermediate A1.

### Intermediate A6: (3S)-1-(4-Chloro-benzenesulfonyl)-piperidine-3-carboxylic acid

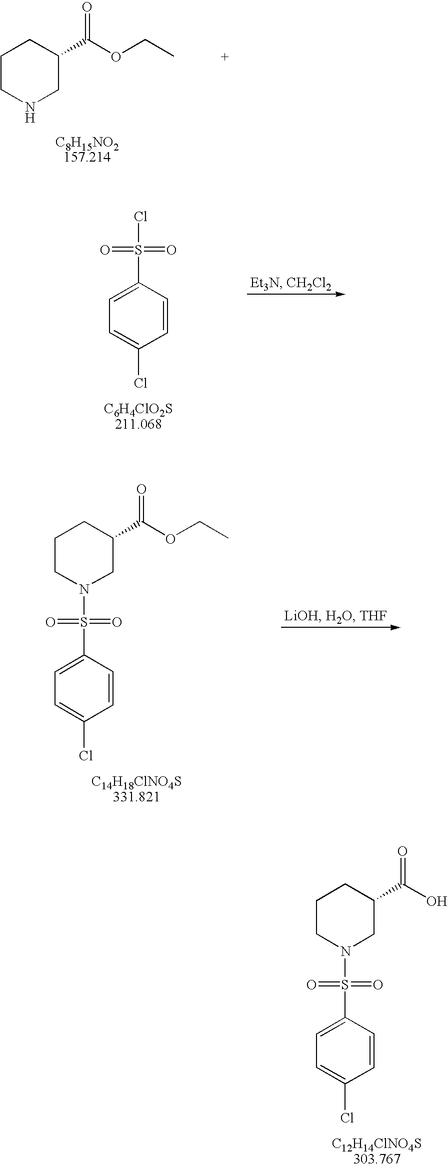

(3S)-1-(4-Chloro-benzenesulfonyl)-piperidine-3-carboxylic acid was prepared from 4-chlorobenzenesulfonyl chloride and (S)-(−)-nipecotic acid ethyl ester (available from Aldrich Chemical Company, Inc., Milwaukee, Wisc.) using the procedure described for the preparation of Intermediate A1.

### Intermediate A7: (3R)-1-(Thiophene-2-sulfonyl)-piperidine-3-carboxylic acid

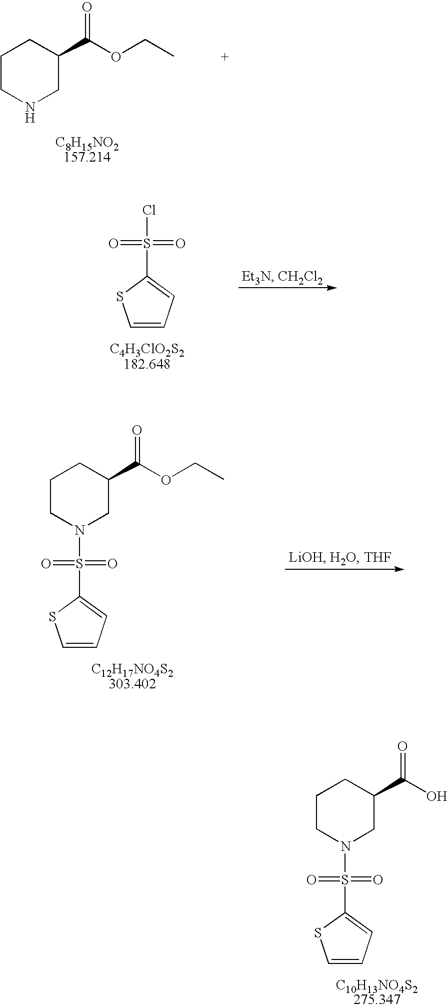

(3R)-1-(Thiophene-2-sulfonyl)-piperidine-3-carboxylic acid was prepared from thiophene-2-sulfonyl chloride and (R)-(+)-nipecotic acid ethyl ester (available from Aldrich Chemical Company, Inc., Milwaukee, Wisc.; 166 mg, 1.1 mmol) using the procedure described for the preparation of Intermediate A1, with the following modification. A second equivalent of thiophene-2-sulfonyl chloride from a different bottle and a second equivalent of triethylamine were added to the reaction mixture because it was determined by NMR that the sulfonyl chloride had hydrolyzed.

### Intermediate A8: (3S)-1-(Thiophene-2-sulfonyl)-piperidine-3-carboxylic acid

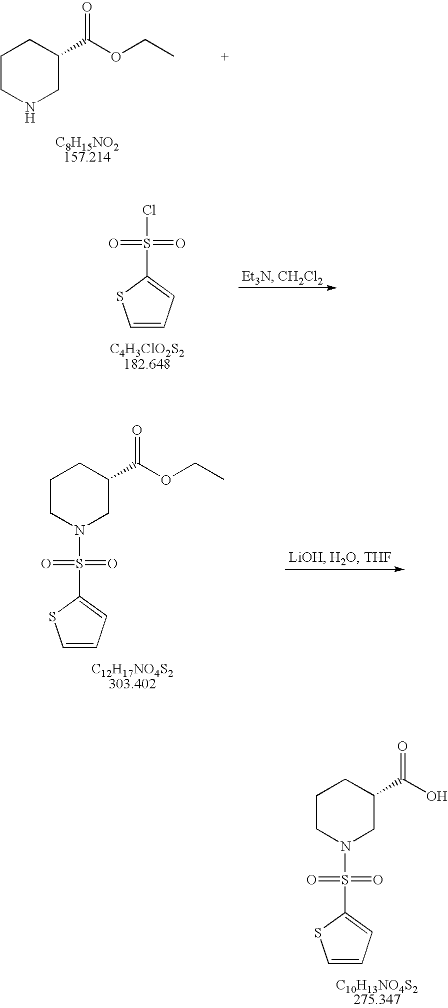

(3S)-1-(Thiophene-2-sulfonyl)-piperidine-3-carboxylic acid was prepared from thiophene-2-sulfonyl chloride and (S)-(+)-nipecotic acid ethyl ester (available from Aldrich Chemical Company, Inc., Milwaukee, Wisc.; 166 mg, 1.1 mmol) using the procedure described for the preparation of Intermediate A1, with the following modification. A second equivalent of thiophene-2-sulfonyl chloride from a different bottle and a second equivalent of triethylamine were added to the reaction mixture because it was determined by NMR that the sulfonyl chloride had hydrolyzed.

### Intermediate B1: 2-Methyl-cyclopentylamine hydrochloride

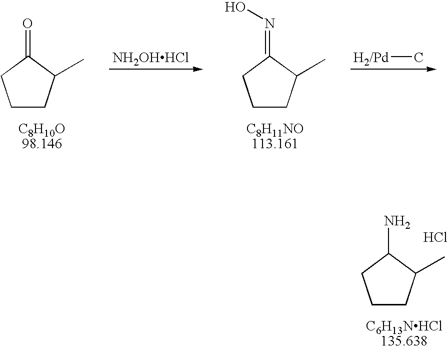

### Step 1. 2-Methylcyclopentanone oxime

A solution of 2-methylcyclopentanone (11 mL, 100 mmol), hydroxylamine hydrochloride (17.76 g, 250 mmol), and triethylamine (42.5 mL, 300 mmol) in ethanol (150 mL) was heated at reflux overnight. The solvent was evaporated and the residue was diluted with water and acidified to pH 1. The mixture was extracted three times with ethyl acetate, and the combined organic layers were washed with water and brine, dried (magnesium sulfate), filtered and evaporated to give 2-methylcyclopentanone oxime (10 g, 88%) as a pale yellow oil.

### Step 2. 2-Methyl-cyclopentylamine hydrochloride

A solution of ethanolic HCl was prepared by adding acetyl chloride (2 mL) to ethanol (100 mL) at 5 degrees, then removing the cooling bath and allowing the solution to stir for 1 h at room temperature. 2-Methylcyclopentanone oxime (from Step 1, 550 mg) was added to this solution along with 10% palladium-on-carbon (two spatulas-full). The mixture was hydrogenated overnight at atmospheric pressure, and then filtered through Celite. The Celite was washed well with ethanol, and the solvents were removed under vacuum. Recrystallization from ethyl acetate gave 2-methyl-cyclopentylamine hydrochloride as a brown solid (330 mg, 50%).

### Part II: Preparation of Preferred Compounds

### Example 1

### (3S)-1-(2-Chloro-benzenesulfonyl)-piperidine-3-carboxylic acid (3-methyl-butyl)-amide

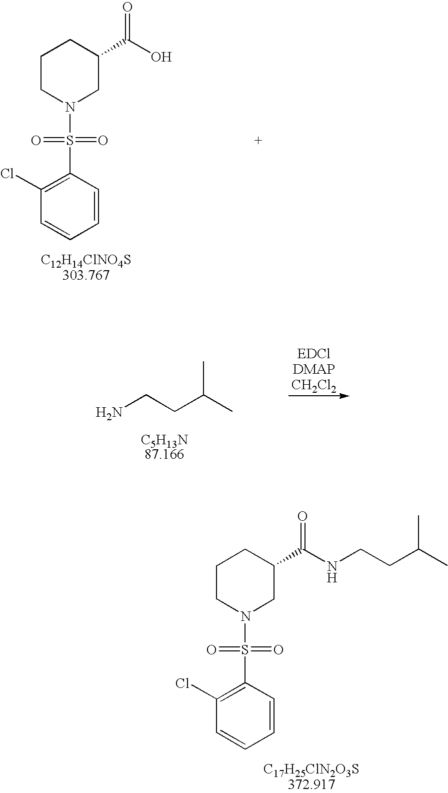

Isoamylamine (0.12 mL, 1.0 mmol) was added to a solution of (3S)-1-(2-chloro-benzenesulfonyl)-piperidine-3-carboxylic acid (of Intermediate A1; 248 mg, 0.8 mmol), 1-hydroxybenzotriazole hydrate (146 mg, 1.1 mmol), N,N-dimethylaminopyridine (202 mg, 1.7 mmol), and 1-(3-dimethylaminopropyl)-3-ethylcarbodiimide hydrochloride (205 mg, 1.1 mmol) in dichloromethane (10 mL). The solution was stirred at room temperature for 5 days, and then diluted with dichloromethane, washed with 1 M HCl (20 mL) and then brine (30 mL), dried (magnesium sulfate), filtered and evaporated. The crude product was purified using an Isco Sg100c RS-40 column, eluting with 15-50% ethyl acetate/hexanes to give (3S)-1-(2-Chloro-benzenesulfonyl)-piperidine-3-carboxylic acid (3-methyl-butyl)-amide (192 mg, 64%) as a white solid. Mass spectrum (ES) MH+=373.

### Example 2

### (3R)-1-(2-Chloro-benzenesulfonyl)-piperidine-3-carboxylic acid (3-methyl-butyl)-amide

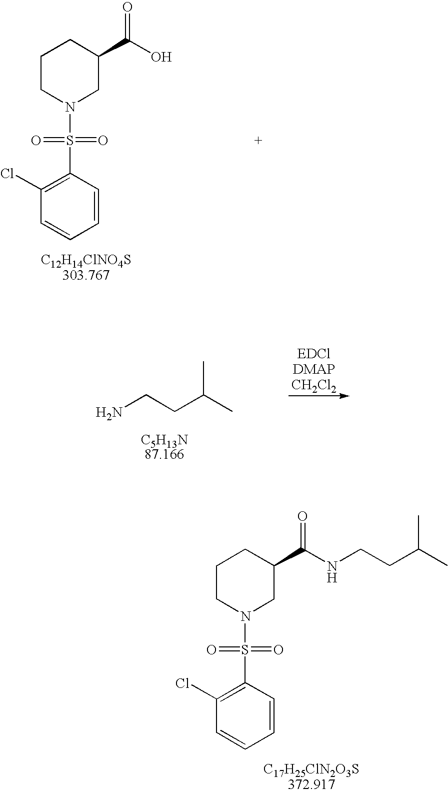

(3R)-1-(2-Chloro-benzenesulfonyl)-piperidine-3-carboxylic acid (3-methyl-butyl)-amide was prepared from (3R)-1-(2-chloro-benzenesulfonyl)-piperidine-3-carboxylic acid (of Intermediate A2) and isoamylamine using the procedure described for the preparation of Example 1. White solid. Yield: 74%. Mass spectrum (ES) MH+=373.

### Example 3

### (rac)-1-(2-Chloro-benzenesulfonyl)-piperidin-3-yl]-(4-hydroxy-piperidin-1-yl)-methanone

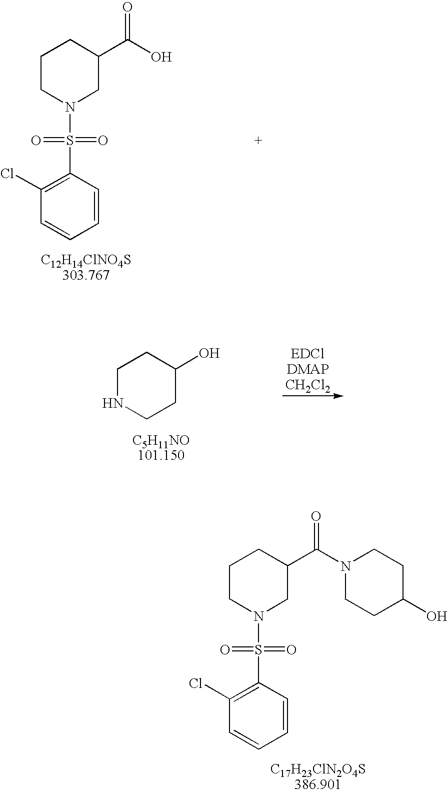

(3R)-1-(2-Chloro-benzenesulfonyl)-piperidine-3-carboxylic acid (3-methyl-butyl)-amide was prepared from (rac)-1-(2-chloro-benzenesulfonyl)-piperidine-3-carboxylic acid (of Intermediate A3) and 4-hydroxypiperidine using the procedure described for the preparation of Example 1. White solid. Yield: 67%. Mass spectrum (ES) MH+=387.

### Example 4

### (3R)-1-(Thiophene-2-sulfonyl)-piperidine-3-carboxylic acid cyclopentylamide

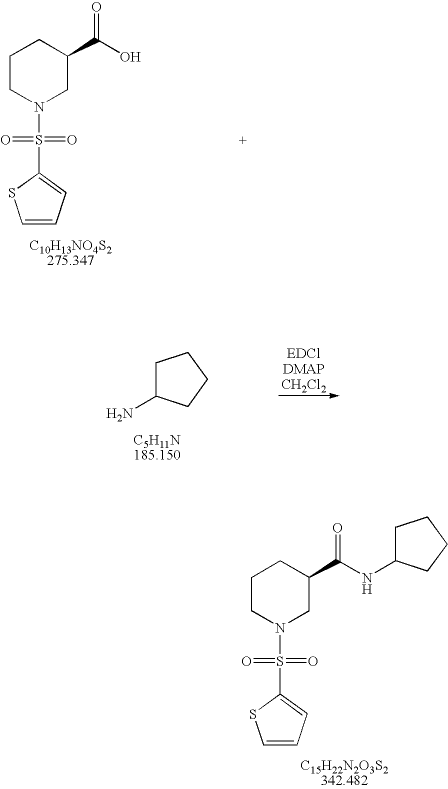

(3R)-1-(Thiophene-2-sulfonyl)-piperidine-3-carboxylic acid cyclopentylamide was prepared from (3R)-1-(thiophene-2-sulfonyl)-piperidine-3-carboxylic acid (of Intermediate A7) and cyclopentylamine using the procedure described for the preparation of Example 1. Off-white solid. Yield: 73%. Mass spectrum (ES) MH+=343.

### Example 5

### (3S)-1-(Thiophene-2-sulfonyl)-piperidine-3-carboxylic acid cyclopentylamide

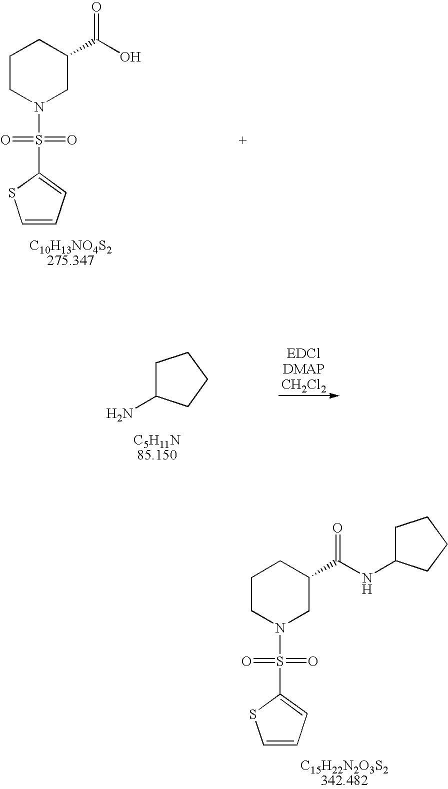

(3S)-1-(Thiophene-2-sulfonyl)-piperidine-3-carboxylic acid cyclopentylamide was prepared from (3S)-1-(thiophene-2-sulfonyl)-piperidine-3-carboxylic acid (of Intermediate A8) and cyclopentylamine using the procedure described for the preparation of Example 1. Off-white solid. Yield: 73%. Mass spectrum (ES) MH+=343.

### Example 6

### (3R)-1-(4-Chloro-benzenesulfonyl)-piperidine-3-carboxylic acid cyclopentylamide

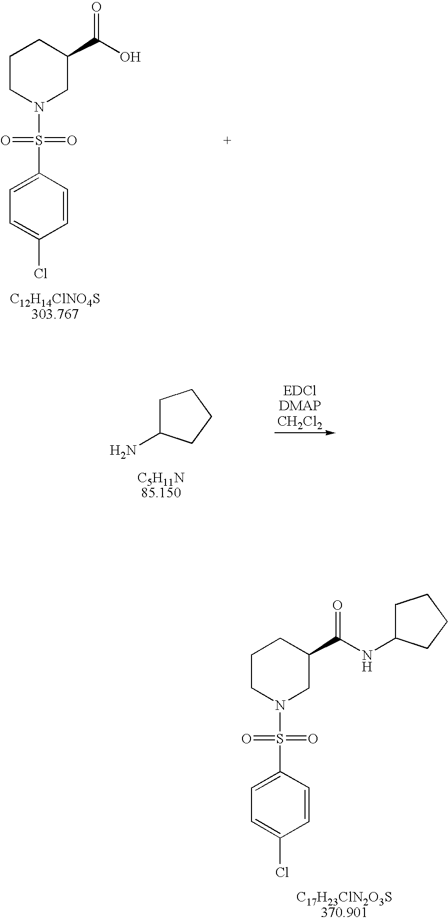

(3R)-1-(4-Chloro-benzenesulfonyl)-piperidine-3-carboxylic acid cyclopentylamide was prepared from (3R)-1-(4-chloro-benzenesulfonyl)-piperidine-3-carboxylic acid (of Intermediate A4) and cyclopentylamine using the procedure described for the preparation of Example 1. White solid. Yield: 80%. Mass spectrum (ES) MH+=371.

### Example 7

### (3S)-1-(4-Chloro-benzenesulfonyl)-piperidine-3-carboxylic acid cyclopentylamide

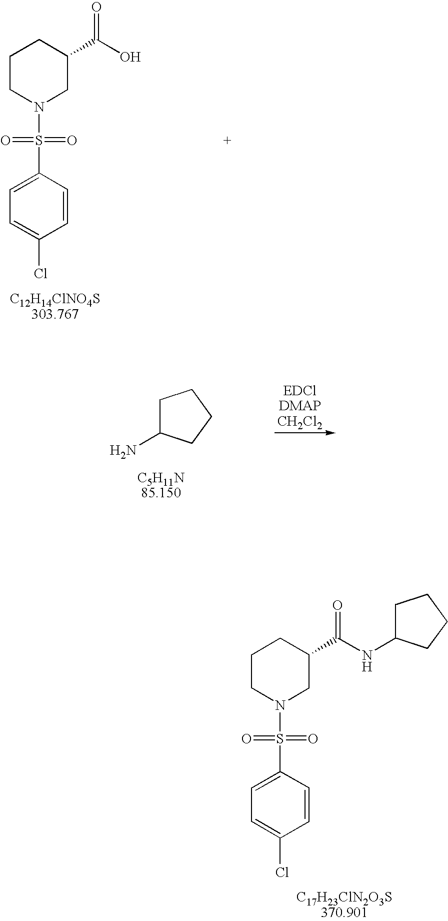

(3S)-1-(4-Chloro-benzenesulfonyl)-piperidine-3-carboxylic acid cyclopentylamide was prepared from (3S)-1-(4-chloro-benzenesulfonyl)-piperidine-3-carboxylic acid (of Intermediate A4) and cyclopentylamine using the procedure described for the preparation of Example 1. White solid. Yield: 69%. Mass spectrum (ES) MH+=371.

### Example 8

### (rac)-[1-(2-Chloro-benzenesulfonyl)-piperidin-3-yl]-(octahydro-quinolin-1-yl)-methanone

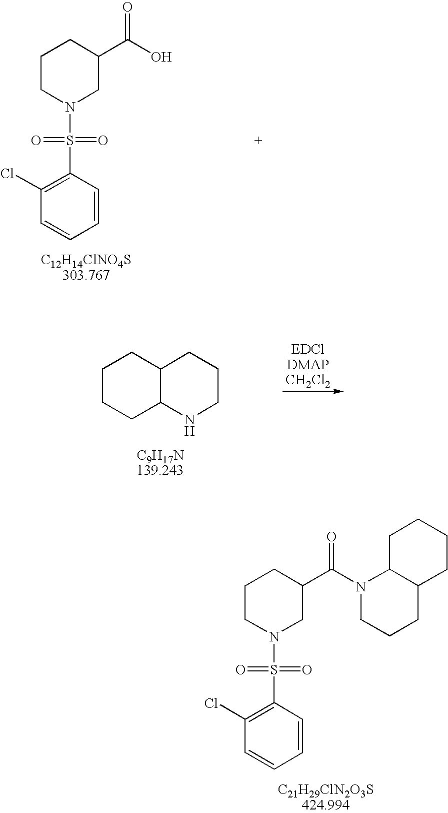

(rac)-[1-(2-Chloro-benzenesulfonyl)-piperidin-3-yl]-(octahydro-quinolin-1-yl)-methanone was prepared from (rac)-1-(2-chloro-benzenesulfonyl)-piperidine-3-carboxylic acid (of Intermediate A3) and decahydroquinoline using the procedure described for the preparation of Example 1. White solid. Yield: 87%. Mass spectrum (ES) MH+=425.

### Example 9

### (rac)-Azepan-1-yl-[1-(2-chloro-benzenesulfonyl)-piperidin-3-yl]-methanone

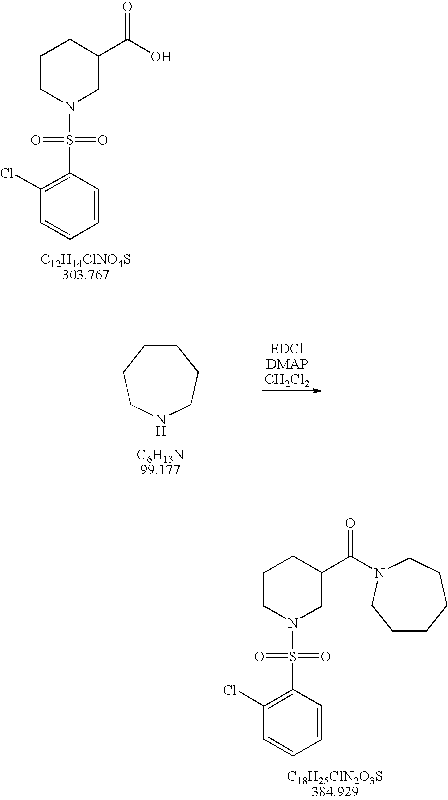

(rac)-Azepan-1-yl-[1-(2-chloro-benzenesulfonyl)-piperidin-3-yl]-methanone was prepared from (rac)-1-(2-chloro-benzenesulfonyl)-piperidine-3-carboxylic acid (of Intermediate A3) and hexamethyleneimine using the procedure described for the preparation of Example 1. White solid. Yield: 65%. Mass spectrum (ES) MH+=385.

### Example 10

### (rac)-[1-(2-Chloro-benzenesulfonyl)-piperidin-3-yl]-(4-methyl-piperidin-1-yl)-methanone

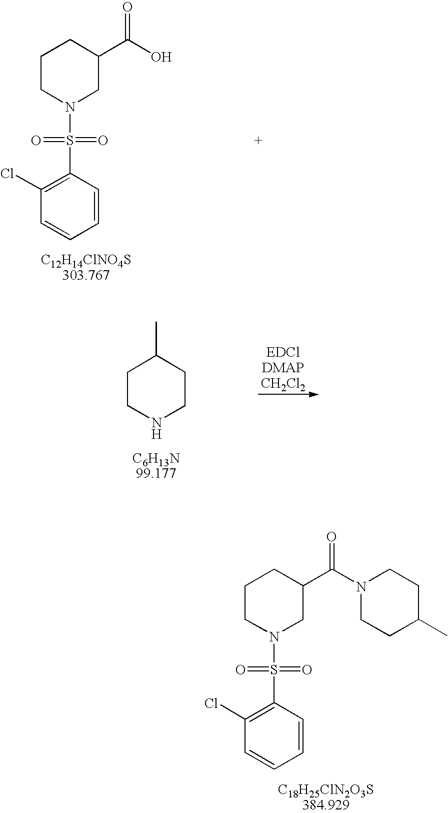

(rac)-[1-(2-Chloro-benzenesulfonyl)-piperidin-3-yl]-(4-methyl-piperidin-1-yl)-methanone was prepared from (rac)-1-(2-chloro-benzenesulfonyl)-piperidine-3-carboxylic acid (of Intermediate A3) and 4-methylpiperidine using the procedure described for the preparation of Example 1. White solid. Yield: 77%. Mass spectrum (ES) MH+=385.

### Example 11

### (rac)-[1-(2-Chloro-benzenesulfonyl)-piperidin-3-yl]-(4,4-dimethyl-piperidin-1-yl)-methanone

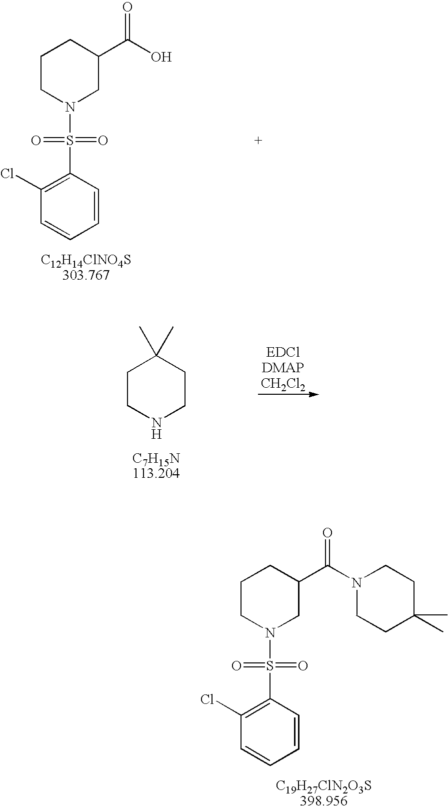

(rac)-[1-(2-Chloro-benzenesulfonyl)-piperidin-3-yl]-(4,4-dimethyl-piperidin-1-methanone was prepared from (rac)-1-(2-chloro-benzenesulfonyl)-piperidine-3-carboxylic acid (of Intermediate A3) and 4,4-dimethylpiperidine (prepared by the reduction of 3,3-dimethyl-glutarimide using lithium aluminum hydride; see D. Hoch and P. Karrer Helv. Chim. Acta 1954, 37, 397) using the procedure described for the preparation of Example 1. White solid. Yield: 82%. Mass spectrum (ES) MH+=399.

### Example 12

### (3S)-1-(2,4-Dichloro-benzenesulfonyl)-piperidine-3-carboxylic acid cyclopentylamide

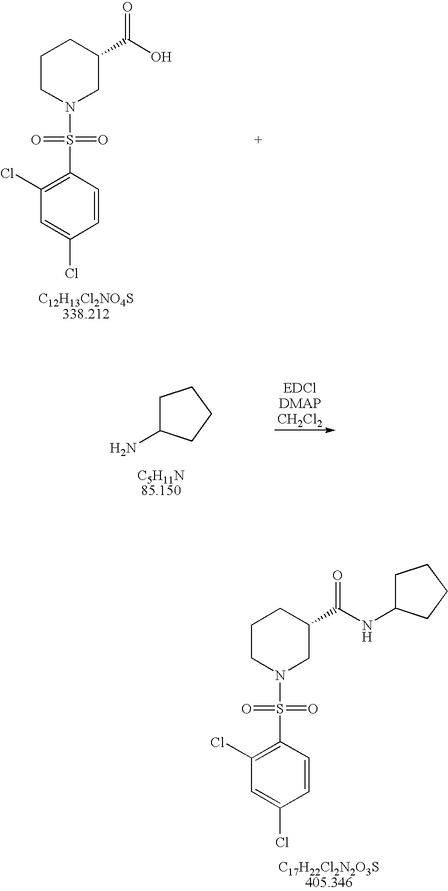

(3S)-1-(2,4-Dichloro-benzenesulfonyl)-piperidine-3-carboxylic acid cyclopentylamide was prepared from (3S)-1-(2,4-dichloro-benzenesulfonyl)-piperidine-3-carboxylic acid (of Intermediate A5) and cyclopentylamine using the procedure described for the preparation of Example 1. White solid. Yield: 60%. Mass spectrum (ES) MH+=405.

### Example 13

### (3S)-1-(2-Chloro-benzenesulfonyl)-piperidine-3-carboxylic acid adamantan-1-ylamide

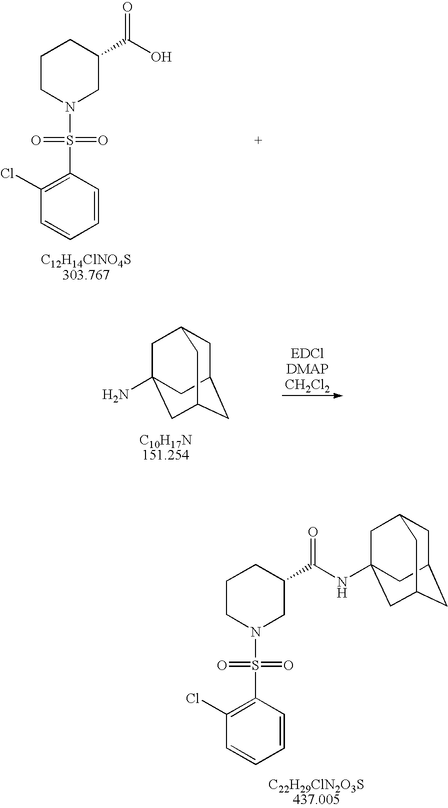

(3S)-1-(2-Chloro-benzenesulfonyl)-piperidine-3-carboxylic acid adamantan-1-ylamide was prepared from (3S)-1-(2-chloro-benzenesulfonyl)-piperidine-3-carboxylic acid (of Intermediate A2) and 1-adamantanamine using the procedure described for the preparation of Example 1. White solid. Yield: 86%. Mass spectrum (ES) MH+=437.

### Example 14

### (3S)-(7-Aza-bicyclo[2.2.1]hept-7-yl)-[1-(2-chloro-benzenesulfonyl)-piperidin-3-yl]-methanone

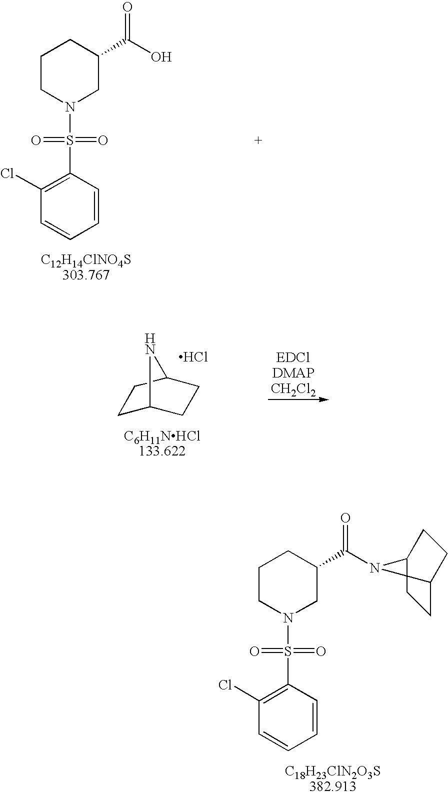

(3 S)-(7-Aza-bicyclo[2.2.1]hept-7-yl)-[1-(2-chloro-benzenesulfonyl)-piperidin-3-yl]-methanone was prepared from (3S)-1-(2-chloro-benzenesulfonyl)-piperidine-3-carboxylic acid (of Intermediate A2) and 7-aza-bicyclo[2.2.1]heptane hydrochloride (Tyger Scientific Inc., Ewing, N.J.) using the procedure described for the preparation of Example 1. White solid. Yield: 76%. Mass spectrum (ES) MH+=383.

### Example 15

### (3S)-[1-(2-Chloro-benzenesulfonyl)-piperidin-3-yl]-(octahydro-quinolin-2-yl)-methanone

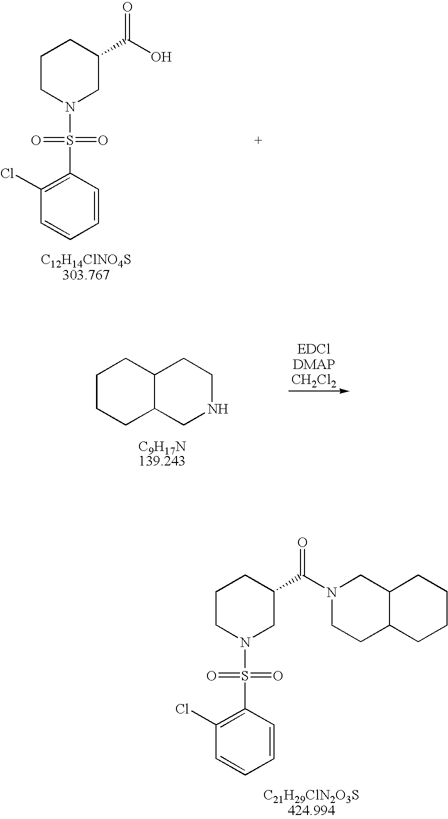

(3S)-[1-(2-Chloro-benzenesulfonyl)-piperidin-3-yl]-(octahydro-quinolin-2-yl)-methanone was prepared from (3S)-1-(2-chloro-benzenesulfonyl)-piperidine-3-carboxylic acid (of Intermediate A2) and decahydroisoquinoline using the procedure described for the preparation of Example 1. White solid. Yield: 84%. Mass spectrum (ES) MH+=425.

### Example 16

### (3S)-(4aR,8aS)-rel-[1-(2-Chloro-benzenesulfonyl)-piperidin-3-yl]-(octahydro-quinolin-2-yl)-methanone

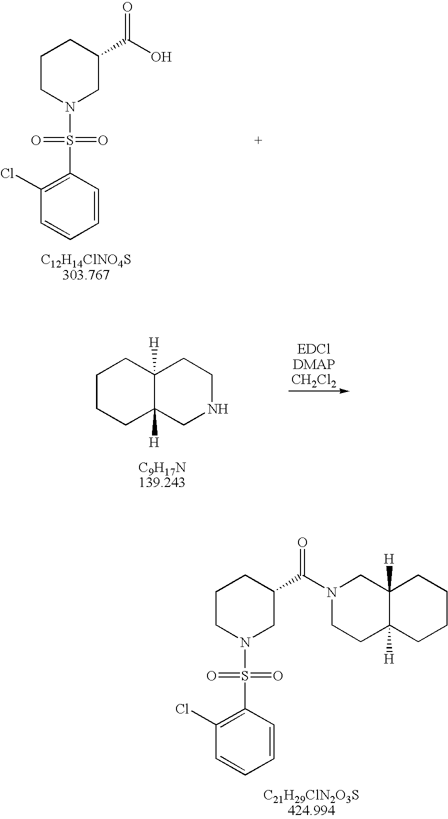

(3S)-(4aR,8aS)-rel-[1-(2-Chloro-benzenesulfonyl)-piperidin-3-yl]-(octahydro-quinolin-2-yl)-methanone was prepared from (3S)-1-(2-chloro-benzenesulfonyl)-piperidine-3-carboxylic acid (of Intermediate A2) and racemic-trans-decahydroisoquinoline (TCI America, Portland, Oreg.) using the procedure described for the preparation of Example 1. White solid. Yield: 90%. Mass spectrum (ES) MH+=425.

### Example 17

### (rac)-[1-(2-Chloro-benzenesulfonyl)-piperidin-3-yl]-morpholin-4-yl-methanone

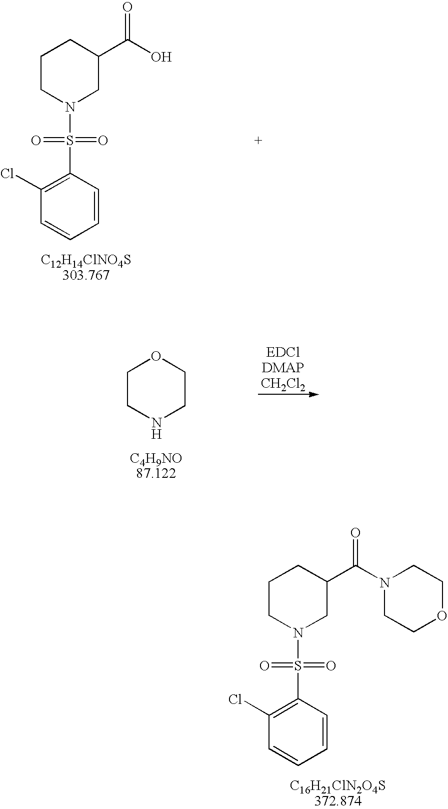

(rac)-[1-(2-Chloro-benzenesulfonyl)-piperidin-3-yl]-morpholin-4-yl-methanone was prepared from (rac)-1-(2-chloro-benzenesulfonyl)-piperidine-3-carboxylic acid (of Intermediate A2) and morpholine using the procedure described for the preparation of Example 1. White foam. Yield: 56%. Mass spectrum (ES) MH+=373.

### Example 18

### (3S)-([1-(2-Chloro-benzenesulfonyl)-piperidin-3-yl-[(cis)-1,3,3a,4,7,7a-hexahydro-isoindol-2-yl]-methanone

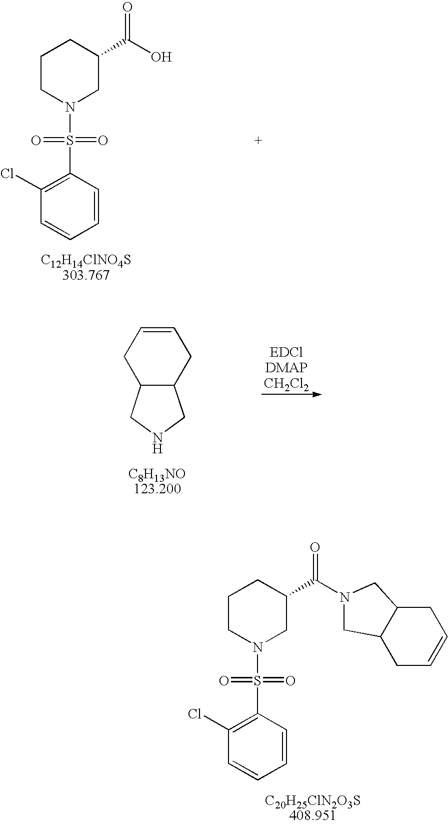

(3S)-([1-(2-Chloro-benzenesulfonyl)-piperidin-3-yl]-[(cis)-1,3,3a,4,7,7a-hexahydro-isoindol-2-yl]-methanone was prepared from (3S)-1-(2-chloro-benzenesulfonyl)-piperidine-3-carboxylic acid (of Intermediate A2) and cis-2,3,3a,4,7,7a-hexahydro-1H-isoindole (prepared by the procedure described in R. D. Otzenberger et al. J. Org. Chem. 1974, 39, 319) using the procedure described for the preparation of Example 1. Pale yellow semi-solid. Yield: 41%. Mass spectrum (ES) MH+=409.

### Example 19

### (3S)-1-(2-Chloro-benzenesulfonyl)-piperidine-3-carboxylic acid (2-methyl-cyclopentyl)-amide

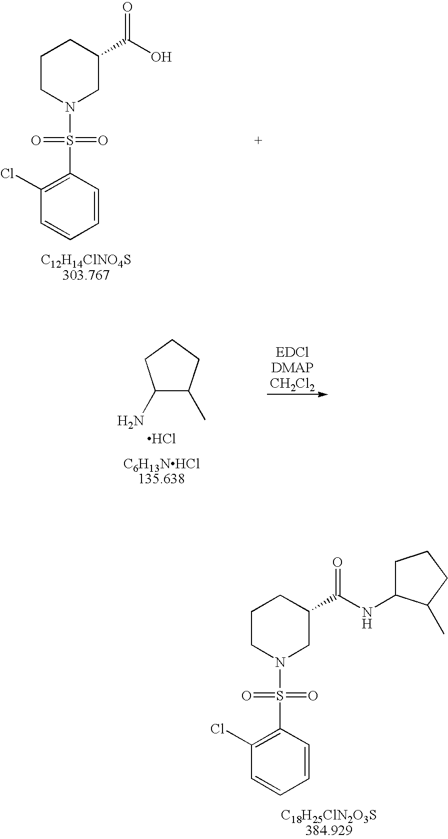

(3S)-1-(2-Chloro-benzenesulfonyl)-piperidine-3-carboxylic acid (2-methyl-cyclopentyl)-amide was prepared from (3S)-1-(2-chloro-benzenesulfonyl)-piperidine-3-carboxylic acid (of Intermediate A2) and 2-methyl-cyclopentylamine hydrochloride (of Intermediate B1) using the procedure described for the preparation of Example 1. Pale white solid. Yield: 35%. Mass spectrum (ES) MH+=385.

### Examples 20 to 201

### Preparation of Compounds of the Invention using Solid-Phase Synthesis

General Procedure 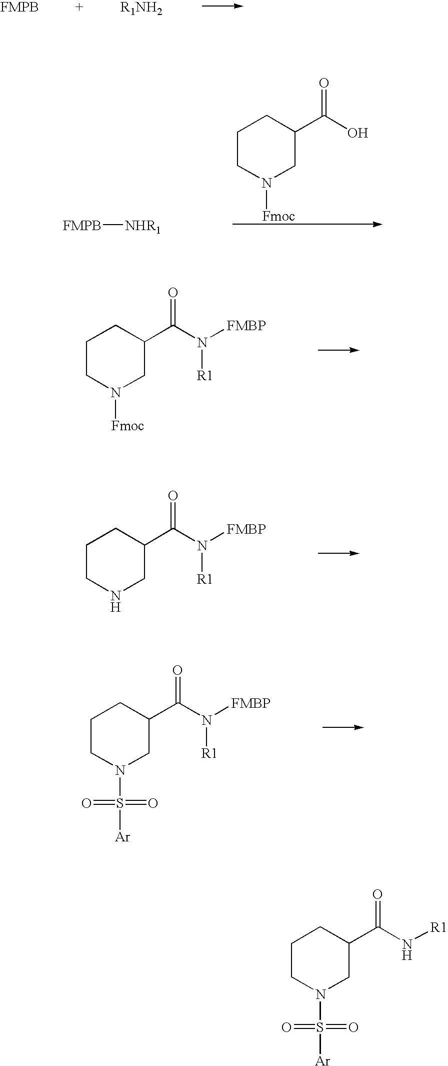
Step 1: Loading of Amine onto FMPB Resin

FMPB resin (Calbiochem-NovaBiochem Corp., San Diego, Calif.; 4-(4-formyl-3-methoxyphenoxy)butyryl AM resin, 50-100 mesh, loading 0.98 mmol/g) was loaded into the IRORI MiniKans (Discovery Partners International, San Diego, Calif.; 85 mg of resin per can). MiniKans to react with the same amine were combined together in one reaction vessel and suspended in a mixture of 1,2-dichloroethane, sodium triacetoxyborohydride (7 eq.), and the appropriate amine (7 eq.) and allowed to react overnight at room temperature. After the reaction solution was drained from each reaction vessel, MiniKans were washed twice with methanol and once with 10% (v/v) triethylamine/dichloromethane. At this stage all MiniKans from different reaction vessels (i.e. reacted with different amines) were combined together and washed sequentially with DMF (once), methanol (once), and dichloromethane (once), and then with DMF (twice), methanol (twice), and dichloromethane (twice). The MiniKans were dried under vacuum overnight.

Step 2: Coupling of Resin-Bound Amine with Fmoc-Nipecotic Acid

The MiniKans from the previous step were suspended in a 50/50 mixture of dichoromethane and DMF, and then N-Fmoc nipecotic acid (Chem-Impex International, Inc., Wood Dale, Ill.; 7 eq.), bromotris(pyrrolydino)phophonium hexafluorophosphate (PyBroP; Calbiochem-NovaBiochem Corp., San Diego, Calif.; 7 eq.) or O-Benzotriazole-N,N,N′,N′-tetramethyl-uronium-hexafluoro-phosphate (HBTU; Alfa Aesar, Ward Hill, Mass.; 7 eq.), and diisopropylethylamine (7 eq.) were added. The reaction was carried out at room temperature overnight. After the reaction solution was drained from the reaction vessel, MiniKans were washed and dried as described above.

Step 3: Capping Procedure

The MiniKans were suspended in DMF solution of acetic anhydride (3 eq.) and diisopropylethylamine (6 eq.) and allowed to react for 2 hours at room temperature. After 2 hours the capping solution was drained and MiniKans were washed and dried as described above.

Step 4: Removal of Fmoc Protective Group

The MiniKans were suspended in 20% (v/v) piperidine/DMF solution and allowed to react for 2 hours at room temperature. After 2 hours the reaction solution was drained and MiniKans were washed and dried as described above.

Step 5: Sulfonylation

The MiniKans were sorted on the IRORI sorter for the sulfonylation reaction. MiniKans to react with the same sulfonyl chloride were combined together in one reaction vessel and suspended in dichloromethane. Then the appropriate sulfonyl chloride (7 eq.) and diisopropylethylamine (7 eq.) were added and the reaction was allowed to go overnight at room temperature. After the reaction solution was drained from each reaction vessel, MiniKans were washed with dichloromethane in each individual reaction vessel. At this stage all MiniKans from different reaction vessels (i.e. reacted with different sulfonyl chlorides) were combined together and washed as described above. The MiniKans were then dried under vacuum overnight.

Step 6: Cleavage of Product from Solid Support

The MiniKans were sorted on the IRORI sorter for cleavage. The final products were cleaved from the solid support on the IRORI cleavage station as follows: TFA/dichloromethane (50/50, v/v; 3 mL) was added to each well. After 3 hours the solution was drained and collected, and each well containing a MiniKan was rinsed with dichloromethane (3 mL) for 20 minutes. The rinse was combined with the solution from the cleavage step and the combined solution was evaporated to dryness on the Genevac. The products were analyzed by LC-MS. Compounds with purity less than 85% were purified as follows:

Description of HT Purification

Samples were dissolved in mixtures of Methanol, ACN and DMSO and purified using the following instruments: Sciex 150 EX Mass Spec, Gilson 215 collector, Shimadzu prep HPLC system, Leap autoinjector. All compounds were purified using TFA buffers LC/MS in the positive ion detection: Solvent (A) 0.05% TFA/H20 (B) 0.035% TFA/ACN, using the appropriate linear gradient mode in 10 minutes, with a C-18 column, 2.0×10 cm eluting at 20 ml/min and mass directed collection

The following compounds were prepared by solid phase synthesis, using the amines and sulfonyl chlorides indicated:

| | | | | | M + H |
|---|---|---|---|---|---|
| Example | Structure | Sulfonyl chloride | Amine | Name | Observed |
| 20 | 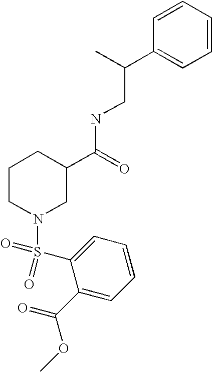 | 2-(Methoxycarbonyl)- benzenesulfonyl chloride | 2-Phenyl- propylamine | 2-[3-(2-Phenyl- propylcarbamoyl)- piperidine-1- sulfonyl]- benzoic acid methyl ester | 445 |
| 21 | 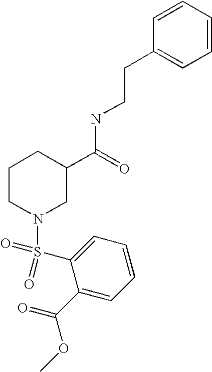 | 2-(Methoxycarbonyl)- benzenesulfonyl chloride | Cyclohexyl- methylamine | 2-[3- (Cyclohexyl- methyl-carbamoyl)- piperidine-1- sulfonyl]- benzoic acid methyl ester | 423 |
| 22 | 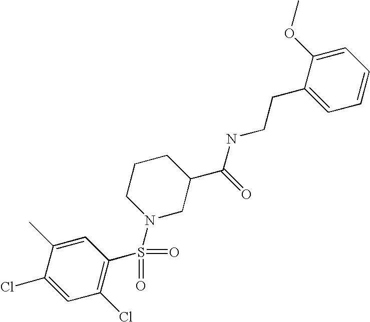 | 2,4-Dichloro-5- methyl- benzenesulfonyl chloride | 2-(2-Methoxy- phenyl)- ethylamine | 1-(2,4-Dichloro- 5-methyl- benzenesulfonyl)- piperidine-3- carboxylic acid [2-(2-methoxy- phenyl)-ethyl]- amide | 485 |
| 23 | 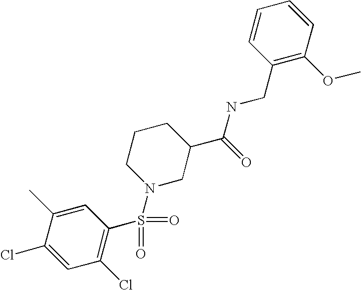 | 2,4-Dichloro-5- methyl- benzenesulfonyl chloride | 2-Methoxy- benzylamine | 1-(2,4-Dichloro- 5-methyl- benzenesulfonyl)- piperidine-3- carboxylic acid 2-methoxy- benzylamide | 471 |
| 24 | 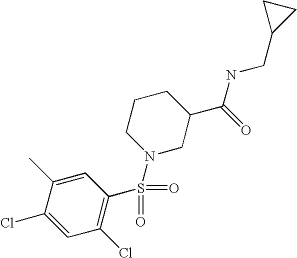 | 2,4-Dichloro-5- methyl- benzenesulfonyl chloride | Cyclopropyl- methylamine | 1-(2,4-Dichloro- 5-methyl- benzenesulfonyl)- piperidine-3- carboxylic acid cyclopropylmethyl- amide | 405 |
| 25 | 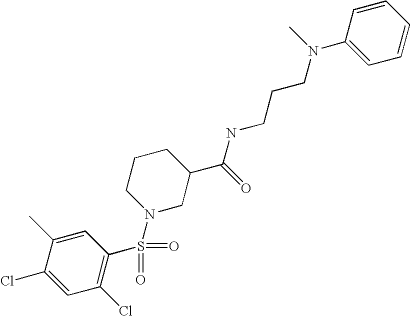 | 2,4-Dichloro-5- methyl- benzenesulfonyl chloride | N-(3- Aminopropyl)-n- methylaniline | 1-(2,4-Dichloro- 5-methyl- benzenesulfonyl)- piperidine-3- carboxylic acid [3-(methyl- phenyl-amino)- propyl]-amide | 498 |
| 26 | 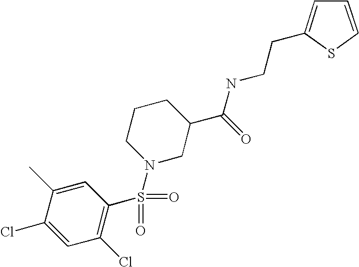 | 2,4-Dichloro-5- methyl- benzenesulfonyl chloride | Thiophene-2- ethylamine | 1-(2,4-Dichloro- 5-methyl- benzenesulfonyl)- piperidine-3- carboxylic acid (2-thiophen-2- yl-ethyl)-amide | 461 |
| 27 | 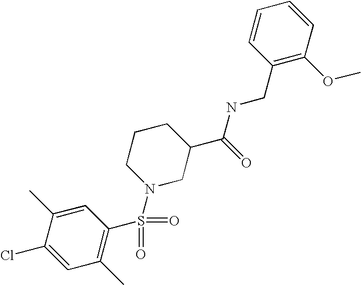 | 2,5-Dimethyl-4- chloro- benzenesulfonyl chloride | 2-Methoxy- benzylamine | 1-(4-Chloro-2,5- dimethyl- benzenesulfonyl)- piperidine-3- carboxylic acid 2-methoxy- benzylamide | 451 |
| 28 | 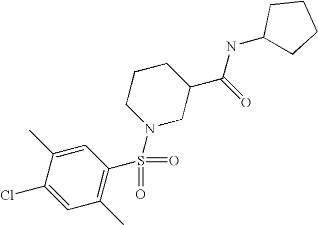 | 2,5-Dimethyl-4- chloro- benzenesulfonyl chloride | Cyclopentylamine | 1-(4-Chloro-2,5- dimethyl- benzenesulfonyl)- piperidine-3- carboxylic acid cyclopentylamide | 399 |
| 29 | 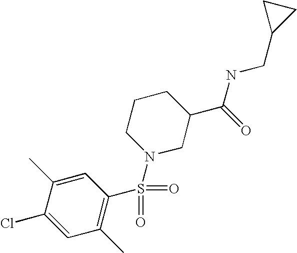 | 2.5-Dimethyl- chloro- benzenesulfonyl chloride | Cyclopropyl- methylamine | 1-(4-Chloro-2,5- dimethyl- benzenesulfonyl)- piperidine-3- carboxylic acid cyclopropylmethyl- amide | 385 |
| 30 | 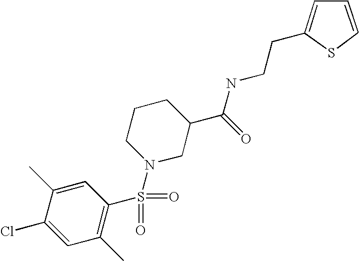 | 2,5-Dimethyl-4- chloro- benzenesulfonyl chloride | Thiophene-2- ethylamine | 1-(4-Chloro-2,5- dimethyl- benzenesulfonyl)- piperidine-3- carboxylic acid (2-thiophen-2- yl-ethyl)-amide | 441 |
| 31 | 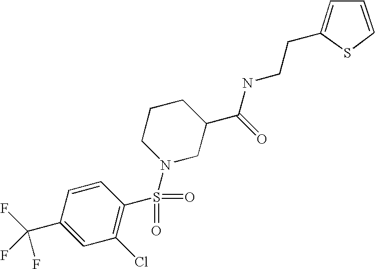 | 2-Chloro-4- trifluoromethyl- bezenesulfonyl chloride | Thiophene-2- ethylamine | 1-(2-Chloro-4- trifluoromethyl- benzenesulfonyl)- piperidine-3- carboxylic acid (2-thiophen-2- yl-ethyl)-amide | 481 |
| 32 | 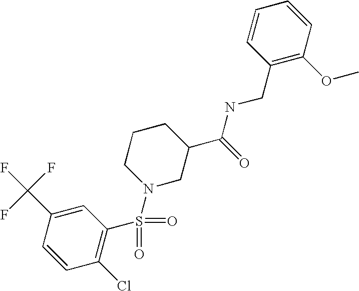 | 2-Chloro-5- trifluoromethyl- benzenesulfonyl chloride | 2-Methoxy- benzylamine | 1-(2-Chloro-5- trifluoromethyl- benzenesulfonyl)- piperidine-3- carboxylic acid 2-methoxy- benzylamide | 491 |
| 33 | 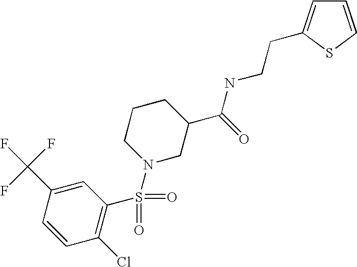 | 2-Chloro-5- trifluoromethyl- benzenesulfonyl chloride | Thiophene-2- ethylamine | 1-(2-Chloro-5- trifluoromethyl- benzenesulfonyl)- piperidine-3- carboxylic acid (2-thiophen-2- yl-ethyl)-amide | 481 |
| 34 | 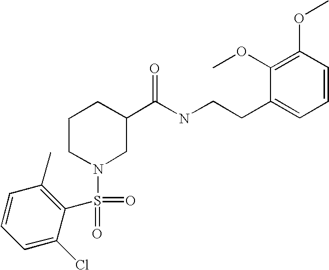 | 2-Chloro-6- methyl- benzenesulfonyl chloride | 2-(2,3- Dimethoxy- phenyl)- ethylamine | 1-(2-Chloro-6- methyl- benzenesulfonyl)- piperidine-3- carboxylic acid [2-(2,3- dimethoxy- phenyl)-ethyl]- amide | 481 |
| 35 | 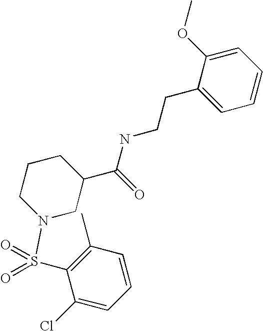 | 2-Chloro-6- methyl- benzenesulfonyl chloride | 2-(2-Methoxy- phenyl)- ethylamine | 1-(2-Chloro-6- methyl- benzenesulfonyl)- piperidine-3- carboxylic acid [2-(2-methoxy- phenyl)-ethyl]- amide | 451 |
| 36 | 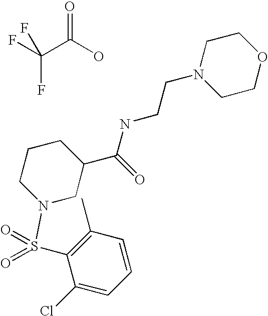 | 2-Chloro-6- methyl- benzenesulfonyl chloride | 2-(Morpholin-4- yl)-ethylamine | 1-(2-Chloro-6- methyl- benzenesulfonyl)- piperidine-3- carboxylic acid (2-morpholin-4- yl-ethyl)-amide; compound with trifluoro-acetic acid | 430 |
| 37 | 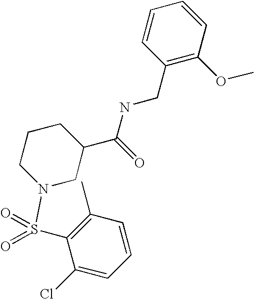 | 2-Chloro-6- methyl- benzenesulfonyl chloride | 2-Methoxy- benzylamine | 1-(2-Chloro-6- methyl- benzenesulfonyl)- piperidine-3- carboxylic acid 2-methoxy- benzylamide | 437 |
| 38 | 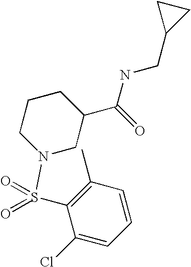 | 2-Chloro-6- methyl- benzenesulfonyl chloride | Cyclopropyl- methylamine | 1-(2-Chloro-6- methyl- benzenesulfonyl)- piperidine-3- carboxylic acid cyclopropylmethyl- amide | 371 |
| 39 | 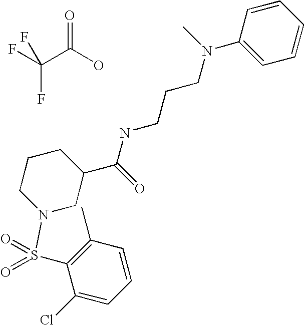 | 2-Chloro-6- methyl- benzenesulfonyl chloride | N-(3- Aminopropyl)-n- methylaniline | 1-(2-Chloro-6- methyl- benzenesulfonyl)- piperidine-3- carboxylic acid [3-(methyl- phenyl-amino)- propyl]-amide; compound with trifluoro-acetic acid | 464 |
| 40 | 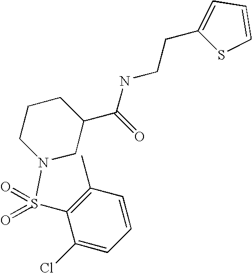 | 2-Chloro-6- methyl- benzenesulfonyl chloride | Thiophene-2- ethylamine | 1-(2-Chloro-6- methyl- benzenesulfonyl)- piperidine-3- carboxylic acid (2-thiophen-2- yl-ethyl)-amide | 427 |
| 41 | 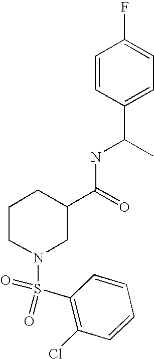 | 2-Chloro- benzenesulfonyl chloride | 1-(4- Fluorophenyl)ethyl- amine | 1-(2-Chloro- benzenesulfonyl)- piperidine-3- carboxylic acid [1-(4-fluoro- phenyl)-ethyl]- amide | 425 |
| 42 | 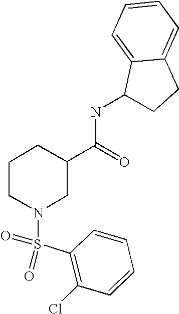 | 2-Chloro- benzenesulfonyl chloride | 1-Aminoindan | 1-(2-Chloro- benzenesulfonyl)- benzenesulfonyl)- piperidine-3- carboxylic acid indan-1-ylamide | 419 |
| 43 | 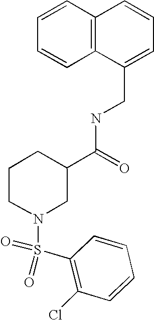 | 2-Chloro- benzenesulfonyl chloride | 1-Naphthalenemethyl- amine | 1-(2-Chloro- benzenesulfonyl)- piperidine-3- carboxylic acid (naphthalen-1- ylmethyl)-amide | 443 |
| 44 | 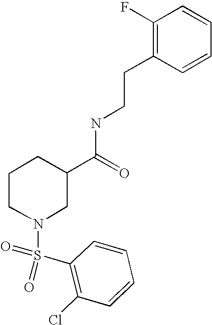 | 2-Chloro- benzenesulfonyl chloride | 2-(2- Fluorophenyl)ethyl- amine | 1-(2-Chloro- benzenesulfonyl)- piperidine-3- carboxylic acid [2-(2-fluoro- phenyl)-ethyl]- amide | 425 |
| 45 | 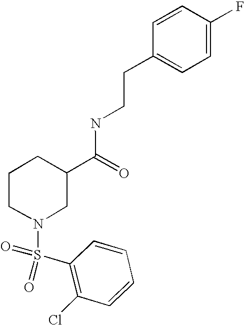 | 2-Chloro- benzenesulfonyl chloride | 2-(4- Fluorophenyl)ethyl- amine | 1-(2-Chloro- benzenesulfonyl)- piperidine-3- carboxylic acid [2-(4-fluoro- phenyl)-ethyl]- amide | 425 |
| 46 | 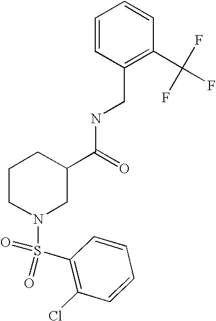 | 2-Chloro- benzenesulfonyl chloride | 2- (Trifluoromethyl)- benzylamine | 1-(2-Chloro- benzenesulfonyl)- piperidine-3- carboxylic acid 2-trifluoromethyl- benzylamide | 461 |
| 47 | 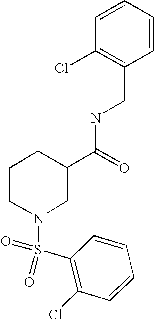 | 2-Chloro- benzenesulfonyl chloride | 2-Chloro- benzylamine | 1-(2-Chloro- benzenesulfonyl)- piperidine-3- carboxylic acid 2-chloro- benzylamide | 427 |
| 48 | 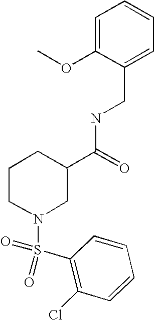 | 2-Chloro- benzenesulfonyl chloride | 2-Methoxy- benzylamine | 1-(2-Chloro- benzenesulfonyl)- piperidine-3- carboxylic acid 2-methoxy- benzylamide | 423 |
| 49 | 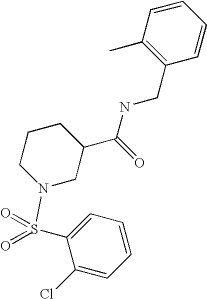 | 2-Chloro- benzenesulfonyl chloride | 2-Methyl-benzylamine | 1-(2-Chloro- benzenesulfonyl)- piperidine-3- carboxylic acid 2-methyl- benzylamide | 407 |
| 50 | 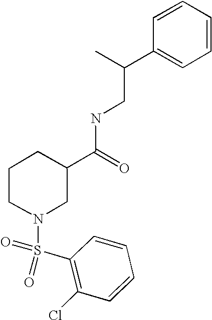 | 2-Chloro- benzenesulfonyl chloride | 2-Phenyl- propylamine | 1-(2-Chloro- benzenesulfonyl)- benzenesulfonyl)- piperidine-3- carboxylic acid (2-phenyl- propyl)-amide | 421 |
| 51 | 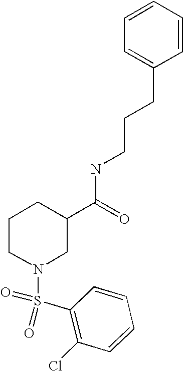 | 2-Chloro- benzenesulfonyl chloride | 3-Phenyl- propylamine | 1-(2-Chloro- benzenesulfonyl)- piperidine-3- carboxylic acid (3-phenyl- propyl)-amide | 421 |
| 52 | 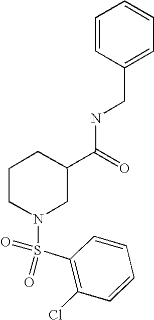 | 2-Chloro- benzenesulfonyl chloride | Benzylamine | 1-(2-Chloro- benzenesulfonyl)- piperidine-3- carboxylic acid benzylamide | 393 |
| 53 | 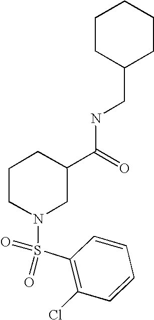 | 2-Chloro- benzenesulfonyl chloride | Cyclohexyl- methylamine | 1-(2-Chloro- benzenesulfonyl)- piperidine-3- carboxylic acid cyclohexylmethyl- amide | 399 |
| 54 | 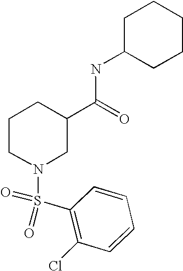 | 2-Chloro- benzenesulfonyl chloride | Cyclohexylamine | 1-(2-Chloro- benzenesulfonyl)- piperidine-3- carboxylic acid cyclohexylamide | 385 |
| 55 | 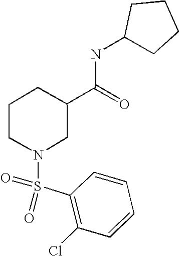 | 2-Chloro- benzenesulfonyl chloride | Cyclopentamine | 1-(2-Chloro- benzenesulfonyl)- piperidine-3- carboxylic acid cyclopentylamide | 371 |
| 56 | 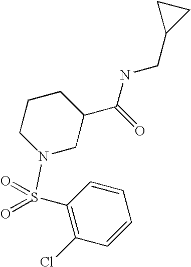 | 2-Chloro- benzenesulfonyl chloride | Cyclopropyl- methylamide | 1-(2-Chloro- benzenesulfonyl)- piperidine-3- carboxylic acid cyclopropylmethyl- amide | 357 |
| 57 | 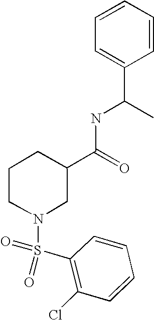 | 2-Chloro- benzenesulfonyl chloride | dl-alpha- Methylbenzyl- amine | 1-(2-Chloro- benzenesulfonyl)- piperidine-3- carboxylic acid (1-phenyl- ethyl)-amide | 407 |
| 58 | 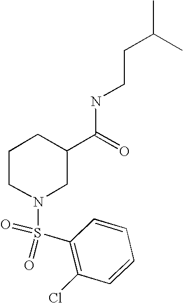 | 2-Chloro- benzenesulfonyl chloride | Isoamylamine | 1-(2-Chloro- benzenesulfonyl)- piperidine-3- carboxylic acid (3-methyl- butyl)-amide | 373 |
| 59 | 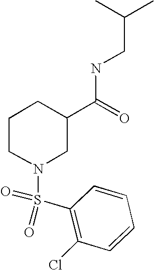 | 2-Chloro- benzenesulfonyl chloride | Isobutylamine | 1-(2-Chloro- benzenesulfonyl)- piperidine-3- carboxylic acid isobutyl-amide | 359 |
| 60 | 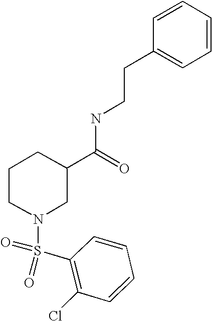 | 2-Chloro- benzenesulfonyl chloride | Phenethylamine | 1-(2-Chloro- benzenesulfonyl)- piperidine-3- carboxylic acid phenethyl-amide | 407 |
| 61 | 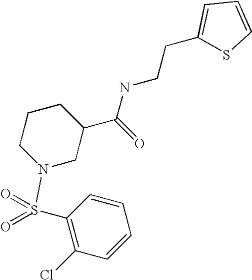 | 2-Chloro- benzenesulfonyl chloride | Thiophene-2- ethylamine | 1-(2-Chloro- benzenesulfonyl)- piperidine-3- carboxylic acid (2-thiophen-2- yl-ethyl)-amide | 413 |
| 62 | 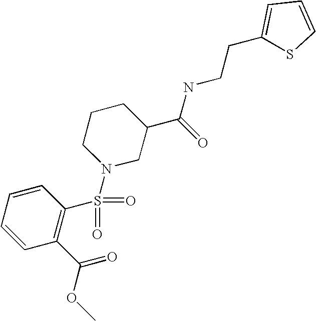 | 2-Methoxycarbonyl- benzenesulfonyl chloride | Thiophene-2- ethylamine | 2-[3-(2- Thiophene-2-yl- ethylcarbamoyl)- piperidine-1- sulfonyl]- benzoic acid methyl ester | 437 |
| 63 | 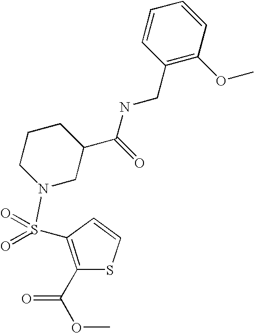 | 2-Methoxycarbonyl- thiophene-3- sulfonyl chloride | 2-Methoxy- benzylamine | 3-[3-(2- Methoxy- benzylcarbamoyl)- piperidine-1- sulfonyl]- thiophene-2- carboxylic acid methyl ester | 453 |
| 64 | 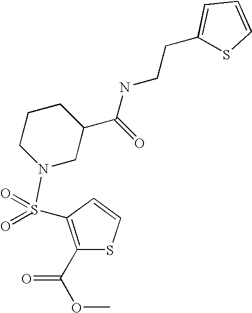 | 2-Methoxycarbonyl- thiophene-3- sulfonyl chloride | Thiophene-2- ethylamine | 3-[3-(2- Thiophen-2-yl- ethylcarbamoyl)- piperidine-1- thiophene-2- carboxylic acid methyl ester | 443 |
| 65 | 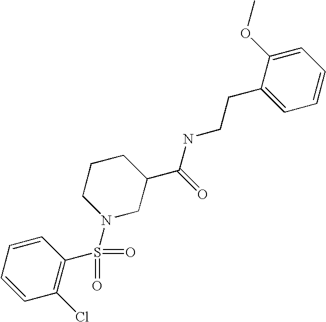 | 2-Methyl- benzenesulfonyl chloride | 2-(2-Methoxy- phenyl)- ethylamine | 1-(Toluene-2- sulfonyl)- piperidine-3- carboxylic acid [2-(2-methoxy- phenyl)-ethyl]- amine | 417 |
| 66 | 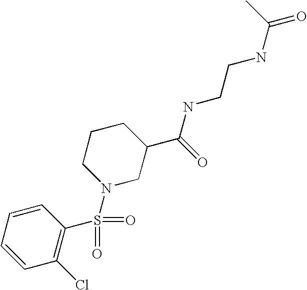 | 2-Methyl- benzenesulfonyl chloride | 2-(Acetamido)- ethylamine | 1-(Toluene-2- sulfonyl)- piperidine-3- carboxylic acid (2-acetylamino- ethyl)-amide | 368 |
| 67 | 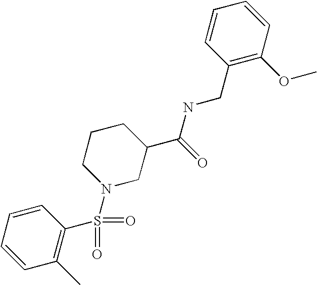 | 2-Methyl-benzenesulfonyl chloride | 2-Methoxy- benzylamine | 1-(Toluene-2- sulfonyl)- piperidine-3- carboxylic acid 2-methoxy- benzylamide | 403 |
| 68 | 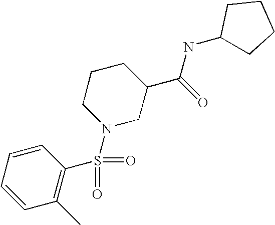 | 2-Methyl- benzenesulfonyl chloride | Cyclopentylamine | 1-(Toluene-2- sulfonyl)- piperidine-3- carboxylic acid cyclopentylamide | 351 |
| 69 | 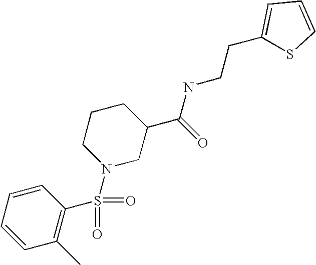 | 2-Methyl- benzenesulfonyl chloride | Thiophene-2- ethylamine | 1-(Toluene-2- sulfonyl)- piperidine-3- carboxylic acid (2-thiophen-2- yl)-ethyl)-amide | 393 |
| 70 | 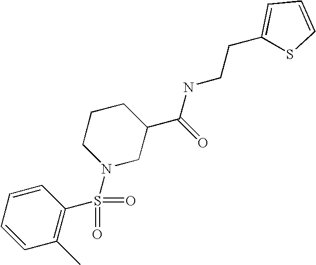 | 2-Naphthylsulfonyl chloride | 2-(2- Fluorophenyl)ethyl- amine | 1-(Naphthalene- 2-sulfonyl)- piperidine-3- carboxylic acid [2-(2-fluoro- phenyl)-ethyl]- amide | 441 |
| 71 | 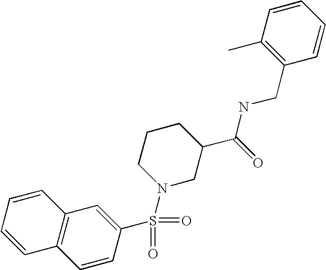 | 2-Naphthylsulfonyl chloride | 2-Methyl- benzylamine | 1-(Naphthalene- 2-sulfonyl)- piperidine-3- carboxylic acid 2-methyl- benzylamide | 423 |
| 72 | 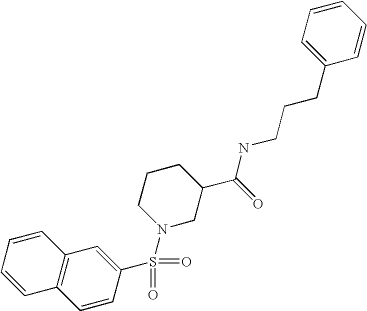 | 2-Naphthylsulfonyl chloride | 3-Phenyl- propylamine | 1-(Naphthalene- 2-sulfonyl)- piperidine-3- carboxylic acid (3-phenyl- propyl)-amide | 437 |
| 73 | 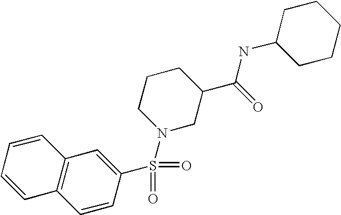 | 2-Naphthylsulfonyl chloride | Cyclohexylamine | 1-(Naphthalene- 2-sulfonyl)- piperidine-3- carboxylic acid cyclohexylamide | 401 |
| 74 | 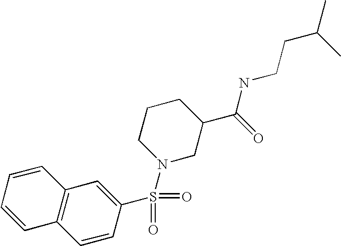 | 2-Naphthylsulfonyl chloride | Isoamylamine | 1-(Naphthalene- 2-sulfonyl)- piperidine-3- carboxylic acid (3-methyl- butyl)-amide | 389 |
| 75 | 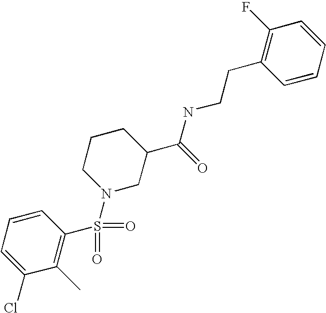 | 3-Chloro-2- methyl- benzenesulfonyl chloride | 2-(2- Fluorophenyl)ethyl- amine | 1-(3-Chloro-2- methyl- benzenesulfonyl)- piperidine-3- carboxylic acid [2-(2-fluoro- phenyl)-ethyl]- amide | 439 |
| 76 | 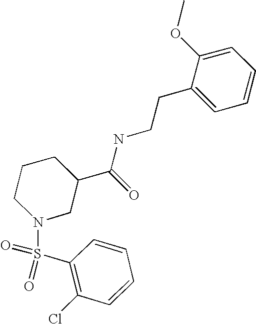 | 3-Chloro-2- methyl- benzenesulfonyl chloride | 2-(2-Methoxy- phenyl)- ethylamine | 1-(3-Chloro-2- methyl- benzenesulfonyl)- piperidine-3- carboxylic acid [2-(2-methoxy- phenyl)-ethyl]- amide | 451 |
| 77 | 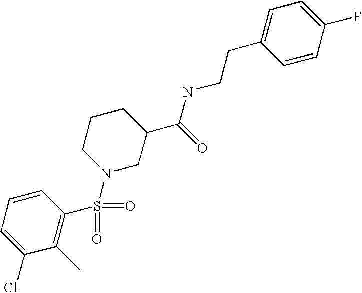 | 3-Chloro-2- methyl- benzenesulfonyl chloride | 2-(4- Fluorophenyl)ethyl- amine | 1-(3-Chloro-2- methyl- benzenesulfonyl)- piperidine-3- carboxylic acid [2-(4-fluoro- phenyl)-ethyl]- amide | 439 |
| 78 | 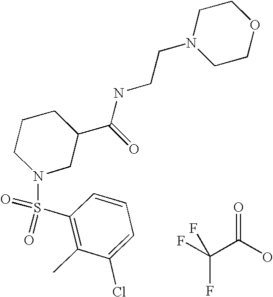 | 3-Chloro-2- methyl- benzenesulfonyl chloride | 2-(Morpholin-4- yl)-ethylamine | 1-(3-Chloro-2- methyl- benzenesulfonyl)- piperidine-3- carboxylic acid (2-morpholin-4- yl-ethyl)-amide; compound with trifluoro-acetic acid | 430 |
| 79 | 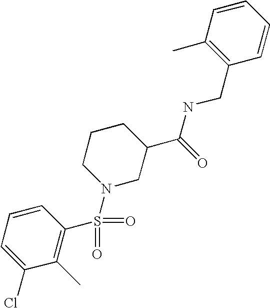 | 3-Chloro-2- methyl- benzenesulfonyl chloride | 2-Methyl- benzylamine | 1-(3-Chloro-2- methyl- benzenesulfonyl)- piperidine-3- carboxylic acid 2-methyl- benzylamide | 421 |
| 80 | 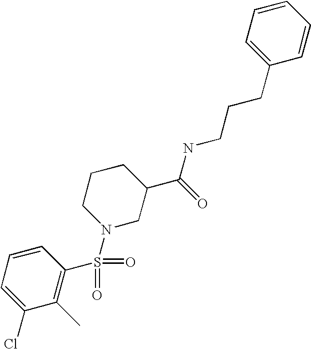 | 3-Chloro-2- methyl- benzenesulfonyl chloride | 3-Phenyl- propylamine | 1-(3-Chloro-2- methyl- benzenesulfonyl)- piperidine-3- carboxylic acid (3-phenyl- propyl)-amide | 435 |
| 81 | 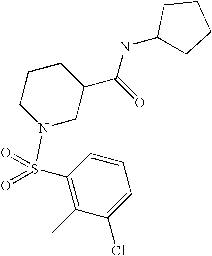 | 3-Chloro-2- methyl- benzenesulfonyl chloride | Cyclopentylamine | 1-(3-Chloro-2- methyl- benzenesulfonyl)- piperidine-3- carboxylic acid cyclopentylamide | 385 |
| 82 | 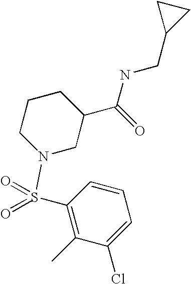 | 3-Chloro-2- methyl- benzenesulfonyl chloride | Cyclopropyl- methylamine | 1-(3-Chloro-2- methyl- benzenesulfonyl)- piperidine-3- carboxylic acid cyclopropylmethyl- amide | 371 |
| 83 | 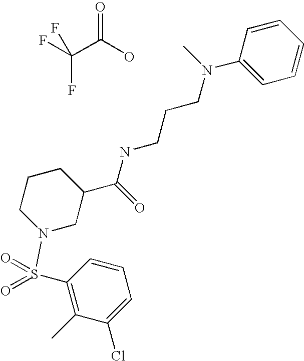 | 3-Chloro-2- methyl- benzenesulfonyl chloride | N-(3- Aminopropyl)-n- methylaniline | 1-(3-Chloro-2- methyl- benzenesulfonyl)- piperidine-3- carboxylic acid [3-(methyl- phenyl-amino)- compound with trifluoro-acetic acid | 464 |
| 84 | 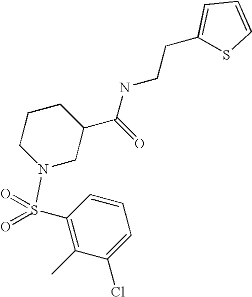 | 3-Chloro-2- methyl- benzenesulfonyl chloride | Thiophene-2- ethylamine | 1-(3-Chloro-2- methyl- benzenesulfonyl)- pipendine-3- carboxylic acid (2-thiophen-2- yl-ethyl)-amide | 427 |
| 85 | 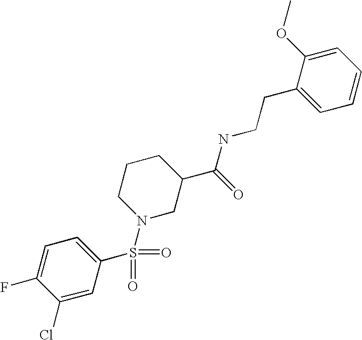 | 3-Chloro-4- fluoro- benzenesulfonyl chloride | 2-(2- Methoxyphenyl)ethyl- amine | 1-(3-Chloro-4- fluoro- benzenesulfonyl)- piperidine-3- carboxylic acid [2-(2-methoxy- phenyl)-ethyl]- amide | 455 |
| 86 | 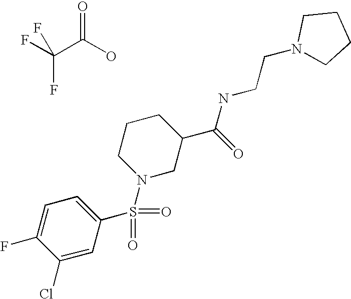 | 3-Chloro-4- fluoro- benzenesulfonyl chloride | 2-(Pyrrolidin-1- yl)-ethylamine | 1-(3-Chloro-4- fluoro- benzenesulfonyl)- piperidine-3- carboxylic acid (2-pyrrolidin-1- yl-ethyl)-amide; compound with trifluoro-acetic acid | 418 |
| 87 | 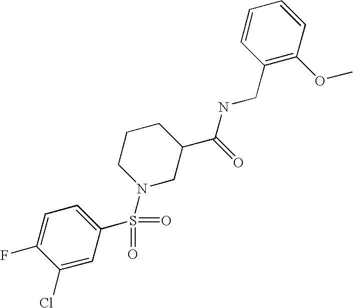 | 3-Chloro-4- fluoro- benzenesulfonyl chloride | 2-Methoxy- benzylamine | 1-(3-Chloro-4- fluoro- benzenesulfonyl)- piperidine-3- carboxylic acid 2-methoxy- benzylamide | 441 |
| 88 | 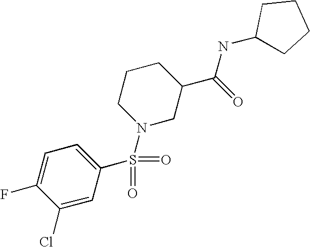 | 3-Chloro-4- fluoro- benzenesulfonyl chloride | Cyclopentylamine | 1-(3-Chloro-4- fluoro- benzenesulfonyl)- piperidine-3- carboxylic acid cyclopentylamide | 389 |
| 89 | 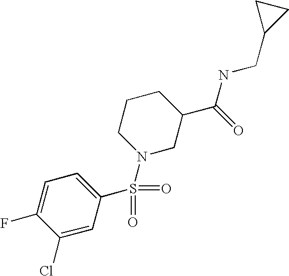 | 3-Chloro-4- fluoro- benzenesulfonyl chloride | Cyclopropyl- methylamine | 1-(3-Chloro-4- fluoro- benzenesulfonyl)- piperidine-3- carboxylic acid cyclopropylmethyl- amide | 375 |
| 90 | 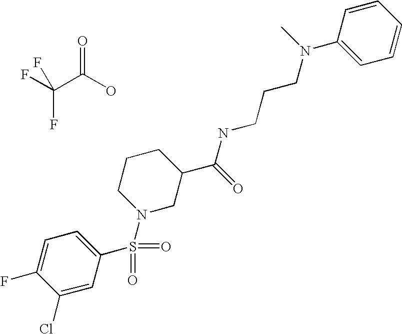 | 3-Chloro-4- fluoro- benzenesulfonyl chloride | N-(3- Aminopropyl)-n- methylaniline | 1-(3-Chloro-4- fluoro- benzenesulfonyl)- piperidine-3- carboxylic acid [3-(methyl- phenyl-amino)- propyl]-amide; compound with trifluoro-acetic acid | 468 |
| 91 | 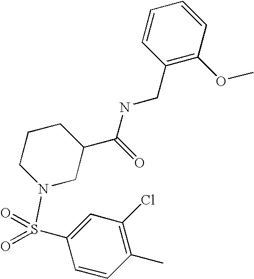 | 3-Chloro-4- methyl- benzenesulfonyl chloride | 2-Methoxy- benzylamine | 1-(3-Chloro-4- methyl- benzenesulfonyl)- methyl- benzenesulfonyl)- piperidine-3- carboxylic acid 2-methoxy- benzylamide | 437 |
| 92 | 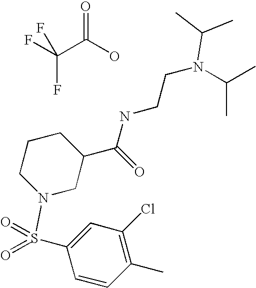 | 3-Chloro-4- methyl- benzenesulfonyl chloride | 3-(N,N- Diisopropylamino)- propylamine | 1-(3-Chloro-4- methyl- benzenesulfonyl)- piperidine-3- carboxylic acid (2-diisopropylamino- ethyl)-amide; compound with trifluoro-acetic acid | 444 |
| 93 | 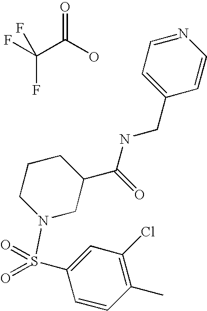 | 3-Chloro-4- methyl- benzenesulfonyl chloride | Pyridine-4- methylamine | 1-(3-Chloro-4- methyl- benzenesulfonyl)- piperidine-3- carboxylic acid (pyridin-4- ylmethyl)- amide; compound with trifluoro-acetic acid | 408 |
| 94 | 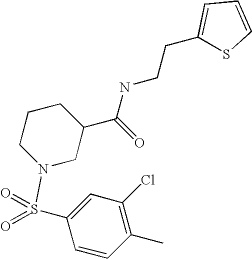 | 3-Chloro-4- methyl- benzenesulfonyl chloride | Thiophene-2- ethylamine | 1-(3-Chloro-4- methyl- benzenesulfonyl)- piperidine-3- carboxylic acid (2-thiophen-2- yl-ethyl)-amide | 427 |
| 95 | 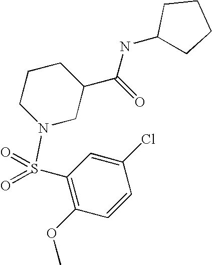 | 3-Chloro-6- methoxy- benzenesulfonyl chloride | Cyclopentylamine | 1-(5-Chloro-2- methoxy- benzenesulfonyl)- piperidine-3- carboxylic acid cyclopentylamide | 401 |
| 96 | 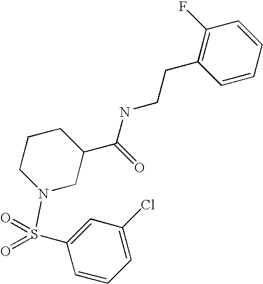 | 3-Chloro- benzenesulfonyl chloride | 2-(2- Fluorophenyl)ethyl- amine | 1-(3-Chloro- benzenesulfonyl)- piperidine-3- carboxylic acid [2-(2-fluoro- phenyl)-ethyl]- amide | 425 |
| 97 | 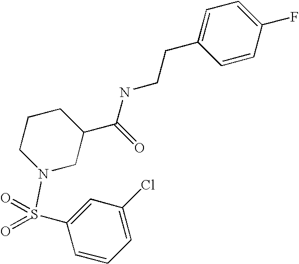 | 3-Chloro- benzenesulfonyl chloride | 2-(4- Fluorophenyl)ethyl- amine | 1-(3-Chloro- benzenesulfonyl)- piperidine-3- carboxylic acid [2-(4-fluoro- phenyl)-ethyl]- amide | 425 |
| 98 | 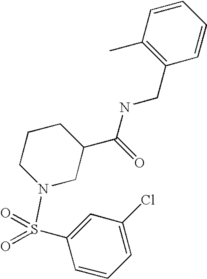 | 3-Chloro- benzenesulfonyl chloride | 2-Methyl- benzylamine | 1-(3-Chloro- benzenesulfonyl)- piperidine-3- carboxylic acid 2-methyl- benzylamide | 407 |
| 99 | 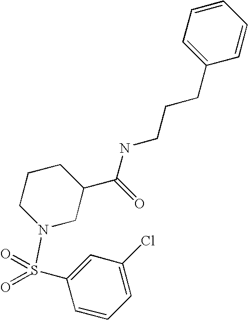 | 3-Chloro- benzenesulfonyl chloride | 3-Phenyl- propylamine | 1-(3-Chloro- benzenesulfonyl)- piperidine-3- carboxylic acid (3-phenyl- propyl)-amide | 421 |
| 100 | 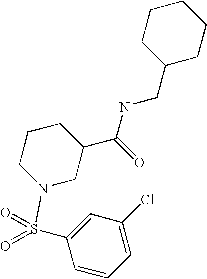 | 3-Chloro- benzenesulfonyl chloride | Cyclohexyl- methylamine | 1-(3-Chloro- benzenesulfonyl)- piperidine-3- carboxylic acid cyclohexylmethyl- amide | 399 |
| 101 | 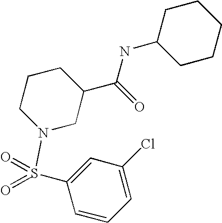 | 3-Chloro- benzenesulfonyl chloride | Cyclohexylamine | 1-(3-Chloro- benzenesulfonyl)- piperidine-3- carboxylic acid cyclohexylamide | 385 |
| 102 | 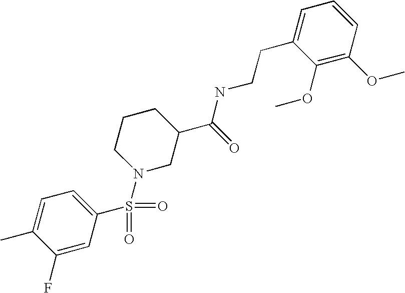 | 3-Fluoro-4- methyl- benzenesulfonyl chloride | 2-(2,3- Dimethoxy- phenyl)- ethylamine | 1-(3-Fluoro-4- methyl- benzenesulfonyl)- piperidine-3- carboxylic acid [2-(2,3- dimethoxy- phenyl)-ethyl]- amide | 465 |
| 103 | 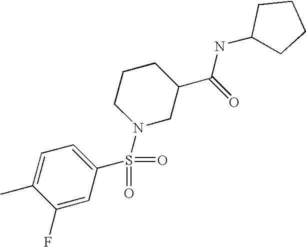 | 3-Fluoro-4- methyl- benzenesulfonyl chloride | Cyclopentylamine | 1-(3-Fluoro-4- methyl- benzenesulfonyl)- piperidine-3- carboxylic acid cyclopentylamide | 369 |
| 104 | 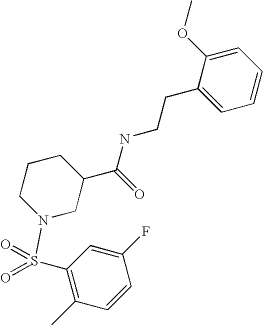 | 3-Fluoro-6- methyl-benzene- sulfonyl chloride | 2-(2-Methoxy- phenyl)- ethylamine | 1-(5-Fluoro-2- methyl- benzenesulfonyl)- piperidine-3- carboxylic acid [2-(2-methoxy- phenyl)-ethyl]- amide | 435 |
| 105 | 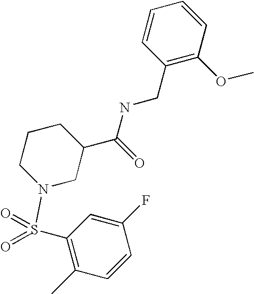 | 3-Fluoro-6- methyl-benzene- sulfonyl chloride | 2-Methoxy- benzylamine | 1-(5-Fluoro-2- methyl- benzenesulfonyl)- piperidine-3- carboxylic acid 2-methoxy- benzylamide | 421 |
| 106 | 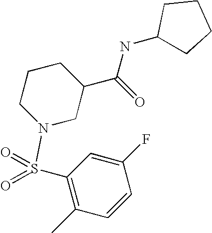 | 3-Fluoro-6- methyl-benzene- sulfonyl chloride | Cyclopentylamine | 1-(5-Fluoro-2- methyl- benzenesulfonyl)- piperidine-3- carboxylic acid cyclopentylamide | 369 |
| 107 | 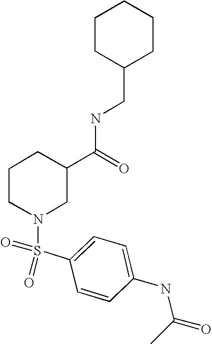 | 4-Acetamido- benzenesulfonyl chloride | Cyclohexyl- methylamine | 1-(4- Acetylamino- benzenesulfonyl)- piperidine-3- carboxylic acid cyclohexylmethyl- amide | 422 |
| 108 | 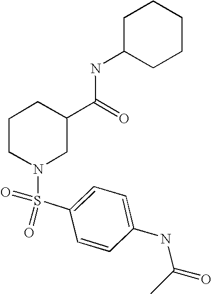 | 4-Acetamido- benzenesulfonyl chloride | cyclohexylamine | 1-(4- Acetylamino- benzenesulfonyl)- piperidine-3- carboxylic acid cyclohexylamide | 408 |
| 109 | 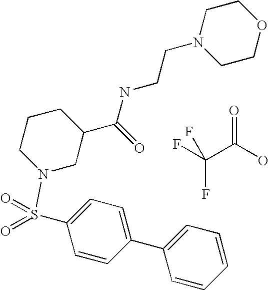 | 4-Bibenzenesulfonyl chloride | 2-(4-Morpholino)- ethylamine | 1-(Biphenyl-4- sulfonyl)- piperidine-3- carboxylic acid (2-morpholin-4- yl-ethyl)-amide; compound with trifluoro-acetic acid | 458 |
| 110 | 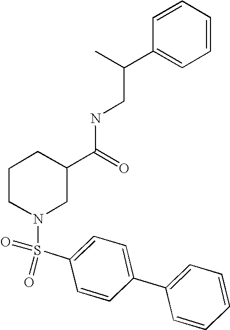 | 4-Bibenzenesulfonyl chloride | 2-Phenyl- propylamine | 1-(Biphenyl-4- sulfonyl)- piperidine-3- carboxylic acid (2-phenyl- propyl)-amide | 463 |
| 111 | 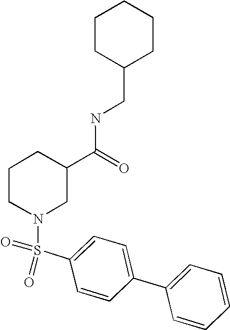 | 4-Bibenzenesulfonyl chloride | Cyclohexyl- methylamine | 1-(Biphenyl-4- sulfonyl)- piperidine-3- carboxylic acid cyclohexylmethyl- amide | 441 |
| 112 | 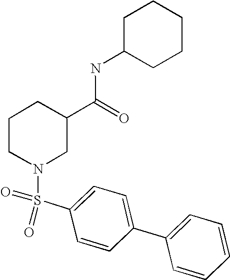 | 4-Bibenzenesulfonyl chloride | Cyclohexylamine | 1-(Biphenyl-4- sulfonyl)- piperidine-3- carboxylic acid cyclohexylamide | 427 |
| 113 | 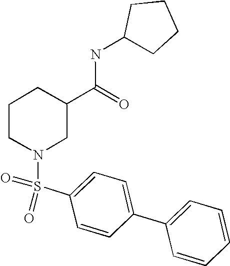 | 4-Bibenzenesulfonyl chloride | Cyclopentamine | 1-(Biphenyl-4- sulfonyl)- piperidine-3- carboxylic acid cyclopentylamide | 413 |
| 114 | 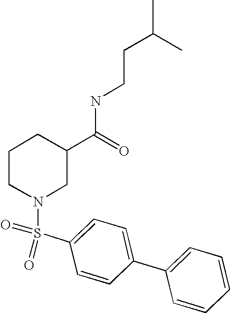 | 4-Bibenzenesulfonyl chloride | Isoamylamine | 1-(Biphenyl-4- sulfonyl)- piperidine-3- carboxylic acid (3-methyl- butyl)-amide | 415 |
| 115 | 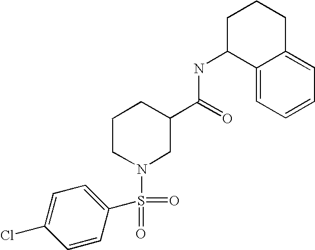 | 4-Chloro- benzenesulfonyl chloride | 1,2,3,4- Tetrahydro-1- naphthylamine | 1-(4-Chloro- benzenesulfonyl)- piperidine-3- carboxylic acid (1,2,3,4- tetrahydro- naphthalen-1- yl)-amide | 433 |
| 116 | 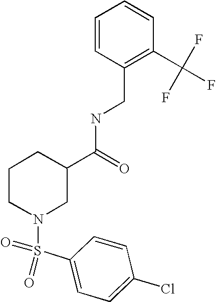 | 4-Chloro- benzenesulfonyl chloride | 2-(TRifluoromethyl)- benzylamine | 1-(4-Chloro- benzenesulfonyl)- piperidine-3- carboxylic acid 2-trifluoromethyl- benzylamide | 461 |
| 117 | 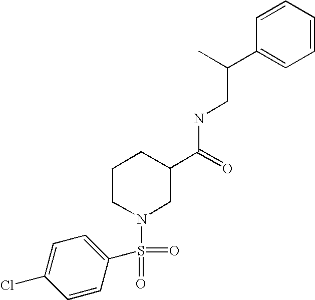 | 4-Chloro- benzenesulfonyl chloride | 2-Phenyl- propylamine | 1-(4-Chloro- benzenesulfonyl)- piperidine-3- carboxylic acid (2-phenyl- propyl)-amide | 421 |
| 118 | 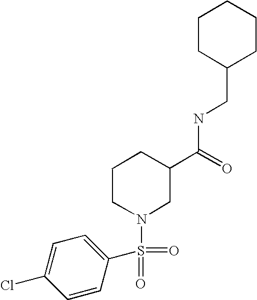 | 4-Chloro- benzenesulfonyl chloride | Cyclohexyl- methylamine | 1-(4-Chloro- benzenesulfonyl)- piperidine-3- carboxylic acid cyclohexylmethyl- amide | 399 |
| 119 | 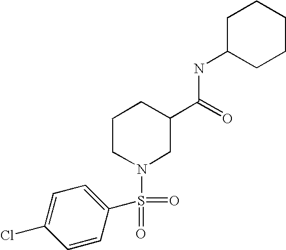 | 4-Chloro- benzenesulfonyl chloride | Cyclohexylamine | 1-(4-Chloro- benzenesulfonyl)- piperidine-3- carboxylic acid cyclohexylamide | 385 |
| 120 | 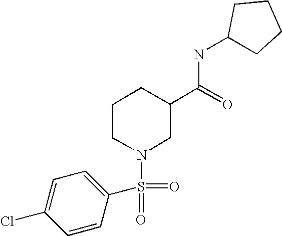 | 4-Chloro- benzenesulfonyl chloride | Cyclopentamine | 1-(4-Chloro- benzenesulfonyl)- piperidine-3- carboxylic acid cyclopentylamide | 371 |
| 121 | 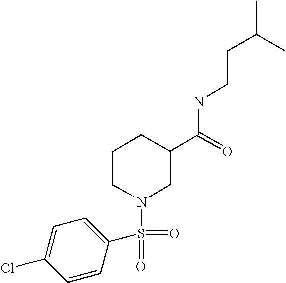 | 4-Chloro- benzenesulfonyl chloride | Isoamylamine | 1-(4-Chloro- benzenesulfonyl)- piperidine-3- carboxylic acid (3-methyl- butyl)-amide | 373 |
| 122 | 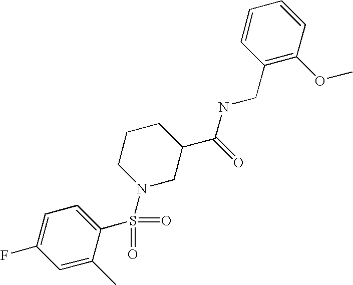 | 4-Fluoro-2- methyl- benzenesulfonyl chloride | 2-Methoxy- benzylamine | 1-(4-Fluoro-2- methyl- benzenesulfonyl)- piperidine-3- carboxylic acid 2-methoxy- benzylamide | 421 |
| 123 | 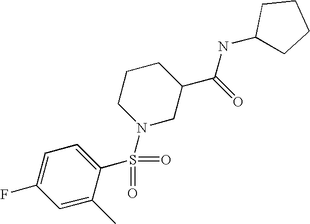 | 4-Fluoro-2- methyl- benzenesulfonyl chloride | Cyclopentylamine | 1-(4-Fluoro-2- methyl- benzenesulfonyl)- piperidine-3- carboxylic acid cyclopentylamide | 369 |
| 124 | 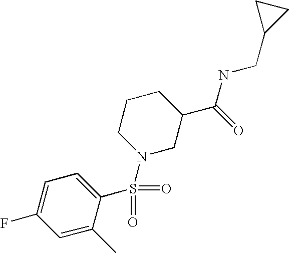 | 4-Fluoro-2- methyl- benzenesulfonyl chloride | Cyclopropyl- methylamine | 1-(4-Fluoro-2- methyl- benzenesulfonyl)- piperidine-3- carboxylic acid cyclopropylmethyl- amide | 355 |
| 125 | 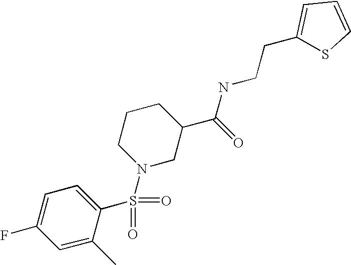 | 4-Fluoro-2- methyl- benzenesulfonyl chloride | Thiophene-2- ethylamine | 1-(4-Fluoro-2- methyl- benzenesulfonyl)- piperidine-3- carboxylic acid (2-thiophen-2- yl-ethyl)-amide | 411 |
| 126 | 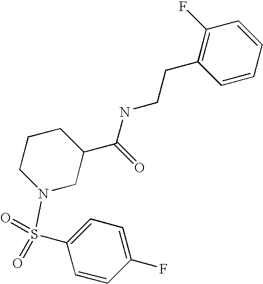 | 4-Fluoro- benzenesulfonyl chloride | 2-(2- Fluorophenyl)ethyl- amine | 1-(4-Fluoro- benzenesulfonyl)- piperidine-3- carboxylic acid [2-(2-fluoro- phenyl)-ethyl]- amide | 409 |
| 127 | 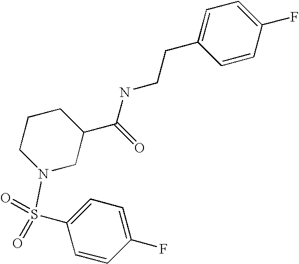 | 4-Fluoro- benzenesulfonyl chloride | 2-(4- Fluorophenyl)ethyl- amine | 1-(4-Fluoro- benzenesulfonyl)- piperidine-3- carboxylic acid [2-(4-fluoro- phenyl)-ethyl]- amide | 409 |
| 128 | 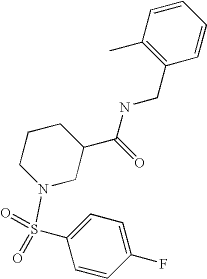 | 4-Fluoro- benzenesulfonyl chloride | 2-Methyl- benzylamine | 1-(4-Fluoro- benzenesulfonyl)- piperidine-3- carboxylic acid 2-methyl- benzylamide | 391 |
| 129 | 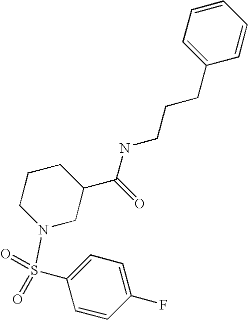 | 4-Fluoro- benzenesulfonyl chloride | 3-Phenyl- propylamine | 1-(4-Fluoro- benzenesulfonyl)- piperidine-3- carboxylic acid (3-phenyl- propyl)-amide | 405 |
| 130 | 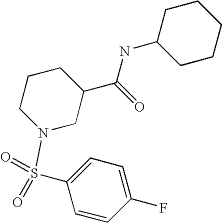 | 4-Fluoro- benzenesulfonyl chloride | Cyclohexylamine | 1-(4-Fluoro- benzenesulfonyl)- piperidine-3- carboxylic acid cyclohexylamide | 369 |
| 131 | 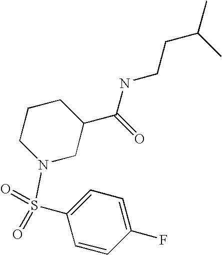 | 4-Fluoro- benzenesulfonyl chloride | Isomaylamine | 1-(4-Fluoro- benzenesulfonyl)- piperidine-3- carboxylic acid (3-methyl- butyl)-amide | 357 |
| 132 | 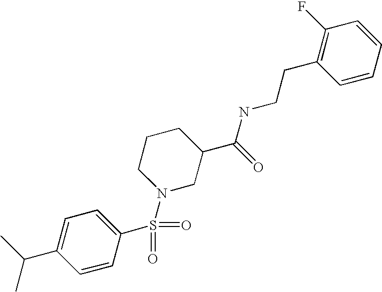 | 4-Isopropyl- benzenesulfonyl chloride | 2-(2- Fluorophenyl)ethyl- amine | 1-(4-Isopropyl- benzenesulfonyl)- piperidine-3- carboxylic acid [2-(2-fluoro- phenyl)-ethyl]- amide | 433 |
| 133 | 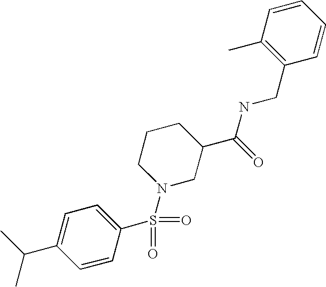 | 4-Isopropyl- benzenesulfonyl chloride | 2-Methyl- benzylamine | 1-(4-Isopropyl- benzenesulfonyl)- piperidine-3- carboxylic acid 2-methyl- benzylamide | 415 |
| 134 | 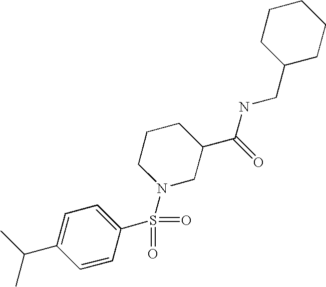 | 4-Isopropyl- benzenesulfonyl chloride | Cyclohexyl- methylamine | 1-(4-Isopropyl- benzenesulfonyl)- piperidine-3- carboxylic acid cyclohexylmethyl- amide | 407 |
| 135 | 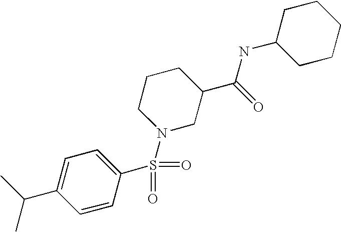 | 4-Isopropyl- benzenesulfonyl chloride | Cyclohexylamine | 1-(4-Isopropyl- benzenesulfonyl)- piperidine-3- carboxylic acid cyclohexylamide | 393 |
| 136 | 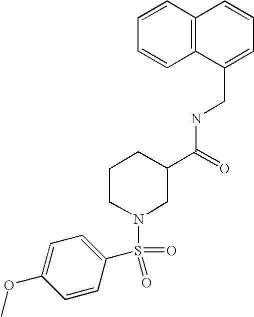 | 4-Methoxy- benzenesulfonyl chloride | 1-Naphthalene- methylamine | 1-(4-Methoxy- benzenesulfonyl)- piperidine-3- carboxylic acid (naphthalen-1- ylmethyl)-amide | 439 |
| 137 | 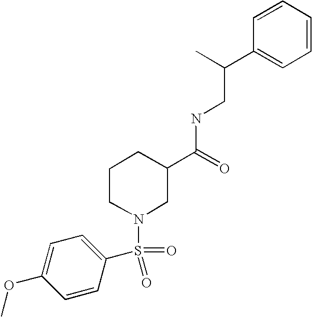 | 4-Methoxy- benzenesulfonyl chloride | 2-phenyl- propylamine | 1-(4-Methoxy- benzenesulfonyl)- piperidine-3- carboxylic acid (2-phenyl- propyl)-amide | 417 |
| 138 | 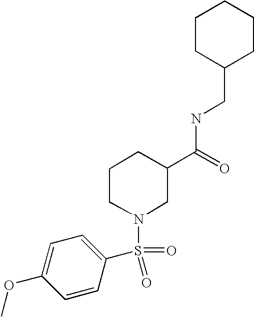 | 4-Methoxy- benzenesulfonyl chloride | Cyclohexyl- methylamine | 1-(4-Methoxy- benzenesulfonyl)- piperidine-3- carboxylic acid cyclohexylmethyl- amide | 395 |
| 139 | 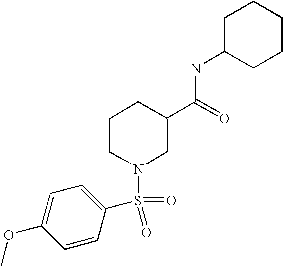 | 4-Methoxy- benzenesulfonyl chloride | Cyclohexylamine | 1-(4-Methoxy- benzenesulfonyl)- piperidine-3- carboxylic acid cyclohexylamine | 381 |
| 140 | 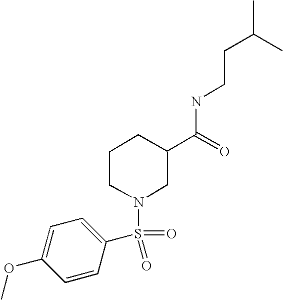 | 4-Methoxy- benzenesulfonyl chloride | Isoamylamine | 1-(4-Methoxy- benzenesulfonyl)- piperidine-3- carboxylic acid (3-methyl- butyl)-amide | 369 |
| 141 | 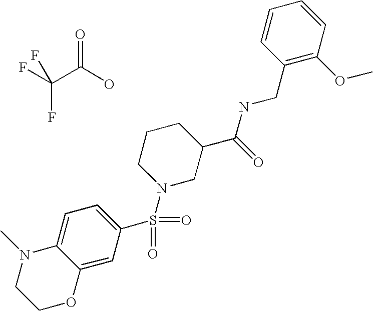 | 4-Methyl-3,4- dihydro-2H- benzp[1,4]oxazone- 7-sulfonyl | 2-Methoxy- benzylamine | 1-(4-Methyl- 3,4-dihydro-2H- benzo[1,4]oxazine- 7-sulfonyl)- piperidine-3- carboxylic acid 2-methoxy- benzylamine; compound with trifluoro-acetic acid | 460 |
| 142 | 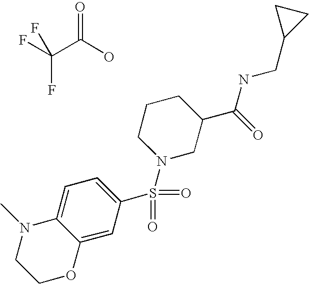 | 4-Methyl-3,4- dihydro-2H- benzo[1,4]oxazine- 7-sulfonyl | Cyclopropyl- methylamine | 1-(4-Methyl- 3,4-dihydro-2H- benzo[1,4]oxazine- 7-sulfonyl)- piperidine-3- carboxylic acid cyclopropylmethyl- amide; compound with trifluoro-acetic acid | 394 |
| 143 | 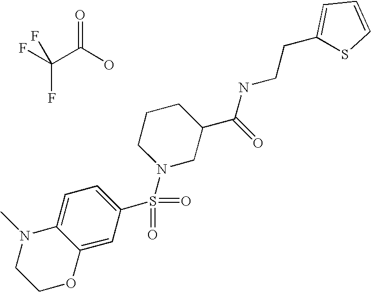 | 4-Methyl-3,4- dihydro-2H- benzo[1,4]oxazine- 7-sulfonyl | Thiophene-2- ethylamine | 1-(4-Methyl- 3,4-dihydro-2H- benzo[1,4]oxazine- 7-sulfonyl)- piperidine-3- carboxylic acid (2-thiophen-2- yl-ethyl)-amide; compound with trifluoro-acetic acid | 450 |
| 144 | 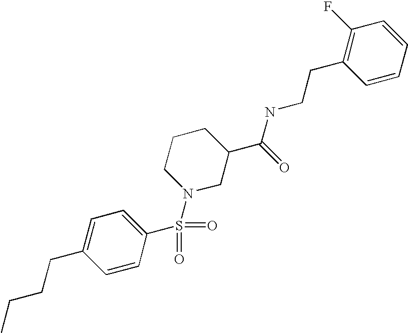 | 4-n-Butyl- benzenesulfonyl chloride | 2-(2- Fluorophenyl)ethyl- amine | 1-(4-Butyl- benzenesulfonyl)- piperidine-3- carboxylic acid [2-(2-fluoro- phenyl)-ethyl]- amide | 447 |
| 145 | 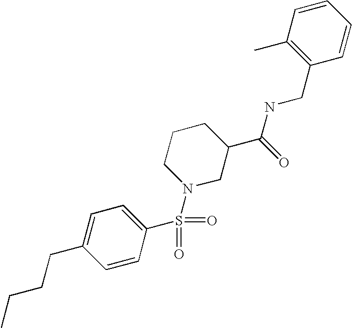 | 4-n-Butyl- benzenesulfonyl chloride | 2-Methyl- benzylamine | 1-(4-Butyl- benzenesulfonyl)- piperidine-3- carboxylic acid 2-methyl- benzylamide | 429 |
| 146 | 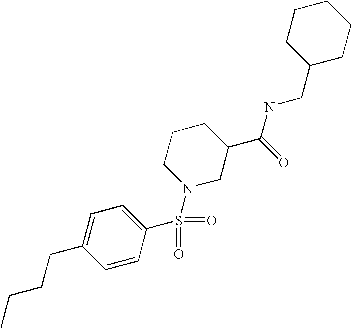 | 4-n-Butyl- benzenesulfonyl chloride | Cyclohexyl- methylamine | 1-(4-Butyl- benzenesulfonyl)- piperidine-3- carboxylic acid cyclohexylmethyl- amide | 421 |
| 147 | 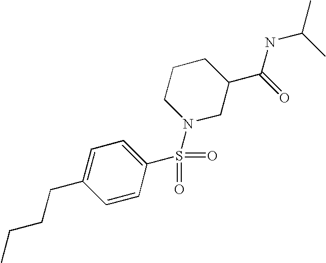 | 4-n-Butyl- benzenesulfonyl chloride | Isopropylamine | 1-(4-Butyl- benzenesulfonyl)- piperidine-3- carboxylic acid isopropylamide | 367 |
| 148 | 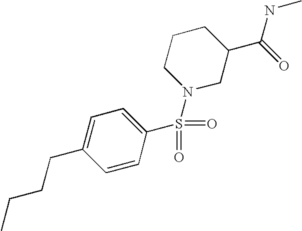 | 4-n-Butyl- benzenesulfonyl chloride | Methylamine | 1-(4-Butyl- benzenesulfonyl)- piperidine-3- carboxylic acid methylamide | 339 |
| 149 | 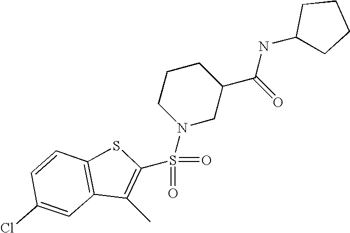 | 5-Chloro-3- methyl- benzo[b]thiophene- 2-sulfonyl chloride | Cyclopentylamine | 1-(5-Chloro-3- methyl- benzo[b]thiophene- 2-sulfonyl)- piperidine-3- carboxylic acid cyclopentylamide | 441 |
| 150 | 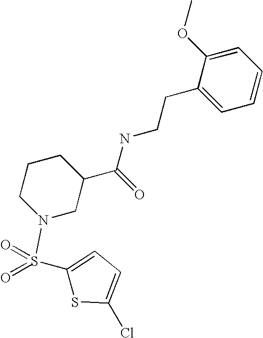 | 5-Chloro- thiophene- sulfonyl chloride | 2-(2-Methoxy- phenyl)- ethylamine | 1-(5-Chloro- thiophene-2- sulfonyl)- piperidine-3- carboxylic acid [2-(2-methoxy- phenyl)-ethyl]- amide | 443 |
| 151 | 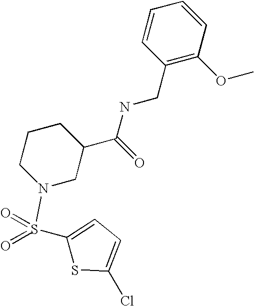 | 5-Chloro- thiophene- sulfonyl chloride | 2-Methoxy- benzylamine | 1-(5-Chloro- thiophene-2- sulfonyl)- piperidine-3- carboxylic acid 2-methoxy- benzylamide | 429 |
| 152 | 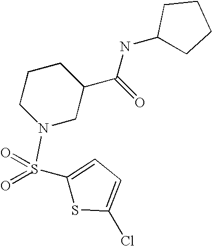 | 5-Chloro- thiophene- sulfonyl chloride | Cyclopentylamine | 1-(5-Chloro- thiophene-2- sulfonyl)- piperidine-3- carboxylic acid cyclopentylamide | 377 |
| 153 | 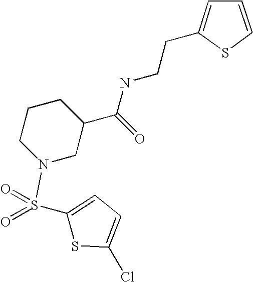 | 5-Chloro- thiophene- sulfonyl chloride | Thiophene-2- ethylamine | 1-(5-Chloro- thiophene-2- sulfonyl)- piperidine-3- carboxylic acid (2-thiophen-2- yl-ethyl)-amide | 419 |
| 154 | 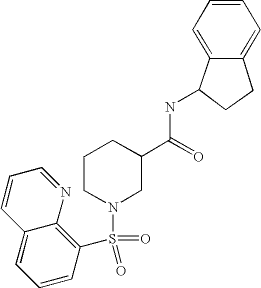 | 8-Quinolinesulfonyl chloride | 1-Aminoindan | 1-(Quinoline-8- sulfonyl)- piperidine-3- carboxylic acid indan-1-ylamide | 436 |
| 155 | 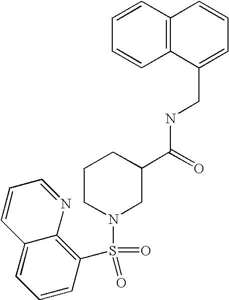 | 8-Quinolinesulfonyl chloride | 1-Naphthalenemethyl- amine | 1-(Quinoline-8- sulfonyl)- piperidine-3- carboxylic acid (naphthalen-1- ylmethyl)-amide | 460 |
| 156 | 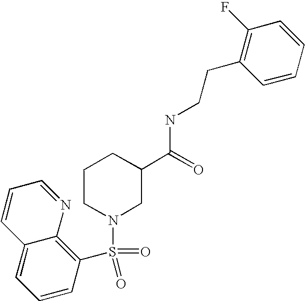 | 8-Quinolinesulfonyl chloride | 2-(2- fluorophenyl)ethyl- amine | 1-(Quinoline-8- sulfonyl)- piperidine-3- carboxylic acid [2-(2-fluoro- phenyl)-ethyl]- amide | 442 |
| 157 | 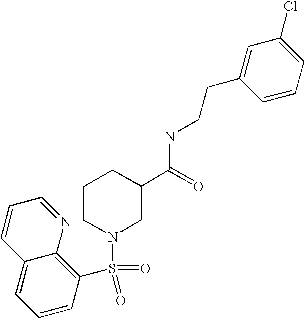 | 8-Quinolinesulfonyl chloride | 2-(3- Chlorophenyl)ethyl- amine | 1-(Quinoline-8- sulfonyl)- piperidine-3- carboxylic acid [2-(3-chloro- phenyl)-ethyl]- amide | 458 |
| 158 | 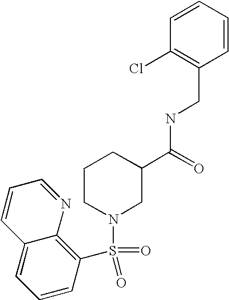 | 8-Quinolinesulfonyl chloride | 2-Chloro- benzylamine | 1-(Quinoline-8- sulfonyl)- piperidine-3- carboxylic acid 2-chloro- benzylamide | 444 |
| 159 | 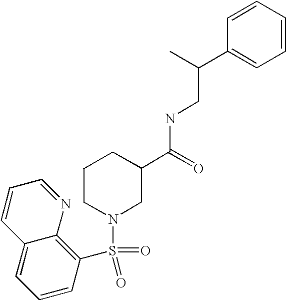 | 8-Quinolinesulfonyl chloride | 2-Phenyl- propylamine | 1-(Quinoline-8- sulfonyl)- piperidine-3- carboxylic acid (2-phenyl- propyl)-amide | 438 |
| 160 | 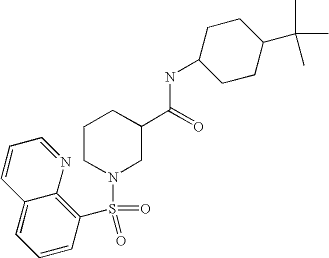 | 8-Quinolinesulfonyl chloride | 4-tert- Butylcyclohexyl- amine | 1-(Quinoline-8- sulfonyl)- piperidine-3- carboxylic acid 4-tert-butyl- cyclohexyl)- amide | 458 |
| 161 | 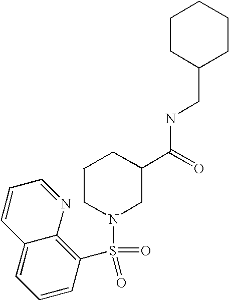 | 8-Quinolinesulfonyl chloride | Cyclohexyl- methylamine | 1-(Quinoline-8- sulfonyl)- piperidine-3- carboxylic acid cyclohexylmethyl- amide | 416 |
| 162 | 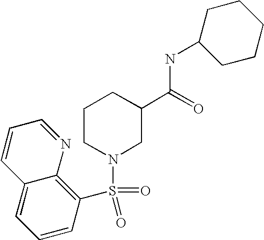 | 8-Quinolinesulfonyl chloride | Cyclohexylamine | 1-(Quinoline-8- sulfonyl)- piperidine-3- carboxylic acid cyclohexylamide | 402 |
| 163 | 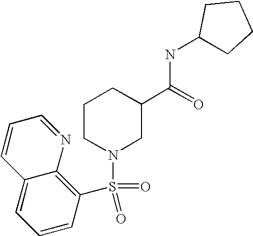 | 8-Quinolinesulfonyl chloride | Cyclopentamine | 1-(Quinoline-8- sulfonyl)- piperidine-3- carboxylic acid cyclopentylamide | 388 |
| 164 | 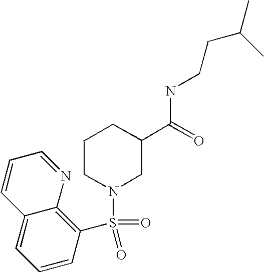 | 8-Quinolinesulfonyl chloride | Isoamylamine | 1-(Quinoline-8- sulfonyl)- piperidine-3- carboxylic acid (3-methyl- butyl)-amide | 390 |
| 165 | 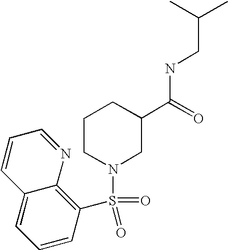 | 8-Quinolinesulfonyl chloride | Isobutylamine | 1-(Quinoline-8- sulfonyl)- piperidine-3- carboxylic acid isobutyl-amide | 376 |
| 166 | 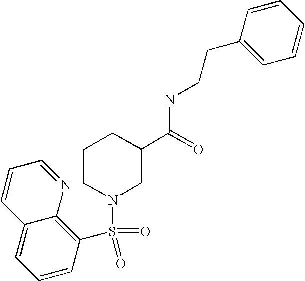 | 8-Quinolinesulfonyl chloride | Phenethylamine | 1-(Quinoline-8- sulfonyl)- piperidine-3- carboxylic acid phenethyl-amide | 424 |
| 167 | 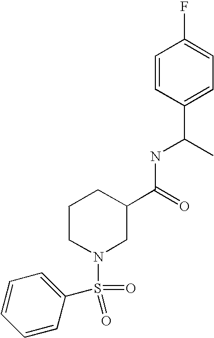 | Benzenesulfonyl chloride | 1-(4-Fluoro- phenyl)ethyl- amine | 1-Benzenesulfonyl)- piperidine-3- carboxylic acid [1-(4-fluoro- phenyl)-ethyl]- amide | 391 |
| 168 | 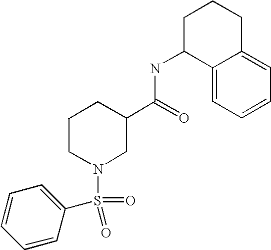 | Benzenesulfonyl chloride | 1,2,3,4- Tetrahydro-1- naphthylamine | 1-Benzenesulfonyl)- piperidine-3- carboxylic acid (1,2,3,4- tetrahydro- naphthalen-1- yl)-amide | 399 |
| 169 | 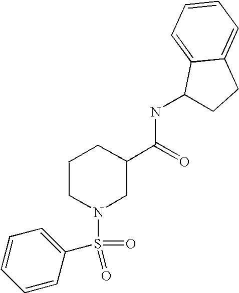 | Benzenesulfonyl chloride | 1-Aminoindan | 1-Benzenesulfonyl- piperidine-3- carboxylic acid indan-1-ylamide | 385 |
| 170 | 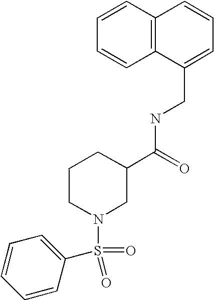 | Benzenesulfonyl chloride | 1-Naphthalene- methylamine | 1-Benzenesulfonyl- piperidine-3- carboxylic acid (naphthalen-1- ylmethyl)-amide | 409 |
| 171 | 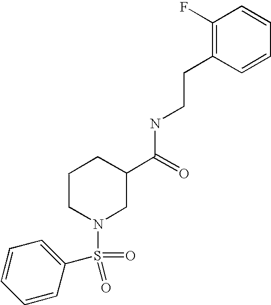 | Benzenesulfonyl chloride | 2-(2-Fluoro- phenyl)ethylamine | 1-Benzenesulfonyl- piperidine-3- carboxylic acid [2-(2-fluoro- phenyl)-ethyl]- amide | 391 |
| 172 | 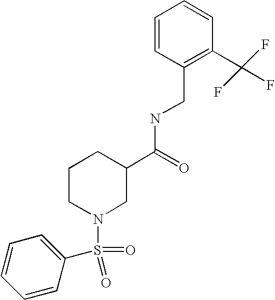 | Benzenesulfonyl chloride | 2-(TRifluoromethyl)- benzylamine | 1-Benzenesulfonyl- piperidine-3- carboxylic acid 2- trifluoromethyl- benzylamide | 427 |
| 173 | 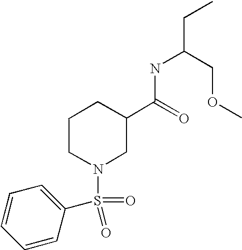 | Benzenesulfonyl chloride | 2-Amino-1- methoxybutane | 1-Benzenesulfonyl- piperidine-3- carboxylic acid (1-methoxymethyl- propyl)-amide | 355 |
| 174 | 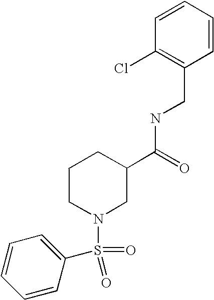 | Benzenesulfonyl chloride | 2-Chloro- benzylamine | 1-Benzenesulfonyl- piperidine-3- carboxylic acid 2-chloro- benzylamide | 393 |
| 175 | 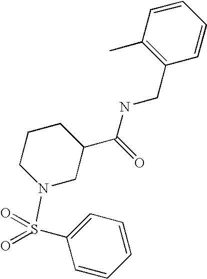 | Benzenesulfonyl chloride | 2-Methyl- benzylamine | 1-Benzenesulfonyl- piperidine-3- carboxylic acid 2-methyl- benzylamide | 373 |
| 176 | 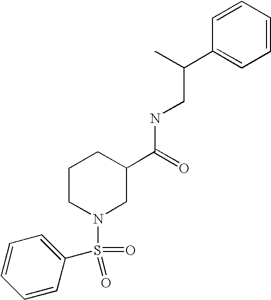 | Benzenesulfonyl chloride | 2-Phenyl- propylamine | 1-Benzenesulfonyl- piperidine-3- carboxylic acid (2-phenyl- propyl)-amide | 387 |
| 177 | 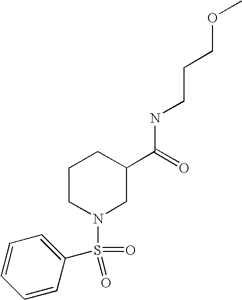 | Benzenesulfonyl chloride | 3-Methoxypropyl- amine | 1-Benzenesulfonyl- piperidine-3- carboxylic acid (3-methoxy- propyl)-amide | 341 |
| 178 | 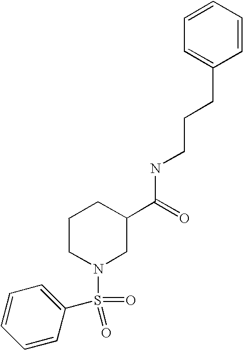 | Benzenesulfonyl chloride | 3-Phenyl- propylamine | 1-Benzenesulfonyl- piperidine-3- carboxylic acid (3-phenyl- propyl)-amide | 387 |
| 179 | 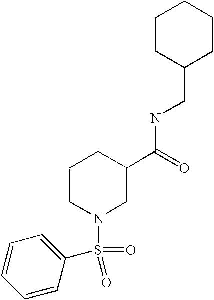 | Benzenesulfonyl chloride | Cyclohexyl- methylamine | 1-Benzenesulfonyl- piperidine-3- carboxylic acid cyclohexylmethyl- amide | 365 |
| 180 | 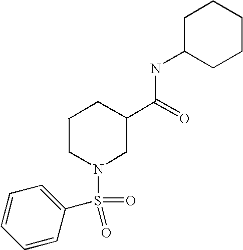 | Benzenesulfonyl chloride | Cyclohexylamine | 1-Benzenesulfonyl- piperidine-3- carboxylic acid cyclohexylamide | 351 |
| 181 | 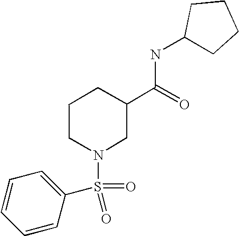 | Benzenesulfonyl chloride | Cyclopentamine | 1-Benzenesulfonyl- piperidine-3- carboxylic acid cyclopentylamide | 337 |
| 182 | 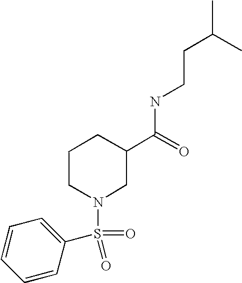 | Benzenesulfonyl chloride | Isoamylamine | 1-Benzenesulfonyl- piperidine-3- carboxylic acid (3-methyl- butyl)-amide | 339 |
| 183 | 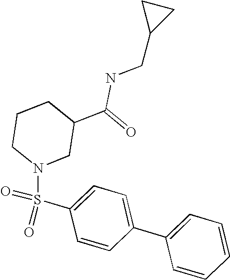 | Biphenyl-4- sulfonyl | Cyclopropyl- methylamine | 1-(Biphenyl-4- sulfonyl)- piperidine-3- carboxylic acid cyclopropylmethyl- amide | 399 |
| 184 | 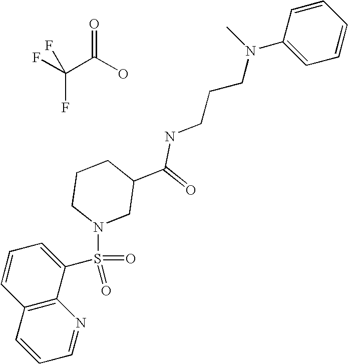 | Quinoline-8- sulfonyl chloride | N-(3- Aminopropyl)-n- methylaniline | 1-(Quinoline-8- sulfonyl)- piperidine-3- carboxylic acid [3-(methyl- phenyl-amino)- propyl]-amide; compound with trifluoro-acetic acid | 467 |
| 185 | 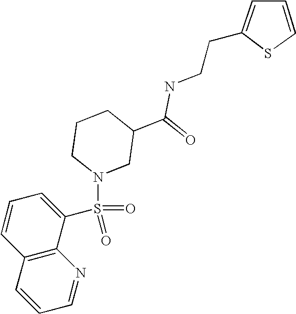 | Quinoline-8- sulfonyl chloride | Thiophene-2- ethylamine | 1-(Quinoline-8- sulfonyl)- piperidine-3- carboxylic acid (2-thiophen-2- yl-ethyl)-amide | 430 |
| 186 | 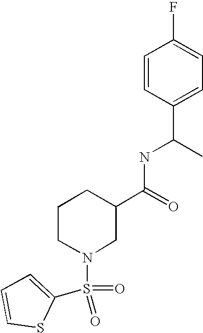 | Thiophene-2- sulfonyl chloride | 1-(4-Fluoro- phenyl)ethylamine | 1-(Thiophene-2- sulfonyl)- piperidine-3- carboxylic acid [1-(4-fluoro- phenyl)-ethyl]- amide | 397 |
| 187 | 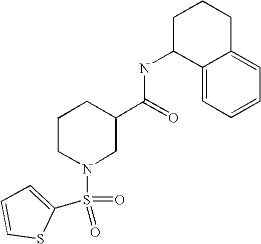 | Thiophene-2- sulfonyl chloride | 1,2,3,4- Tetrahydro-1- naphthylamine | 1-(Thiophene-2- sulfonyl)- piperidine-3- carboxylic acid (1,2,3,4- tetrahydro- naphthalen-1- yl)-amide | 405 |
| 188 | 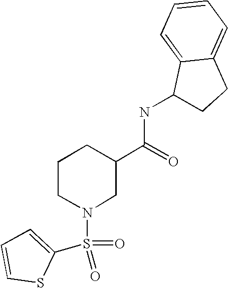 | Thiophene-2- sulfonyl chloride | 1-Aminoindan | 1-(Thiophene-2- sulfonyl)- piperidine-3- carboxylic acid indan-1-ylamide | 391 |
| 189 | 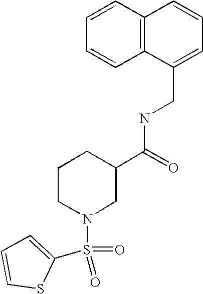 | Thiophene-2- sulfonyl chloride | 1-Naphthalene- methylamine | 1-(Thiophene-2- sulfonyl)- piperidine-3- carboxylic acid (naphthalen-1- ylmethyl)-amide | 415 |
| 190 | 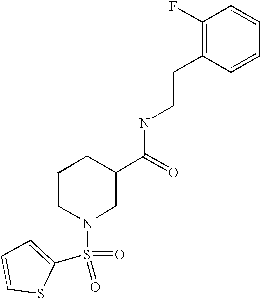 | Thiophene-2- sulfonyl chloride | 2-(2-Fluoro- phenyl)ethylamine | 1-(Thiophene-2- sulfonyl)- piperidine-3- carboxylic acid [2-(2-fluoro- phenyl)-ethyl]- amide | 397 |
| 191 | 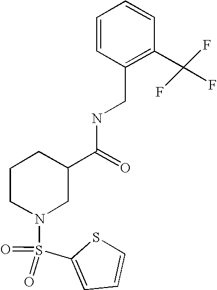 | Thiophene-2- sulfonyl chloride | 2-(Trifluoromethyl)- benzylamine | 1-(Thiophene-2- sulfonyl)- piperidine-3- carboxylic acid 2-trifluoromethyl- benzylamide | 433 |
| 192 | 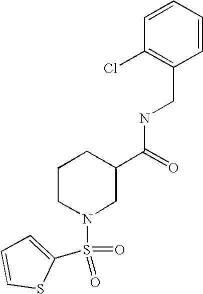 | Thiophene-2- sulfonyl chloride | 2-Chloro- benzylamine | 1-(Thiophene-2- sulfonyl)- piperidine-3- carboxylic acid 2-chloro- benzylamide | 399 |
| 193 | 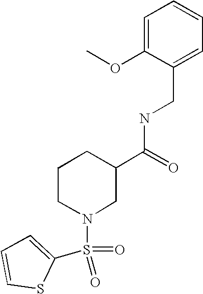 | Thiophene-2- sulfonyl chloride | 2-Methoxy- benzylamine | 1-(Thiophene-2- sulfonyl)- piperidine-3- carboxylic acid 2-methoxy- benzylamide | 395 |
| 194 | 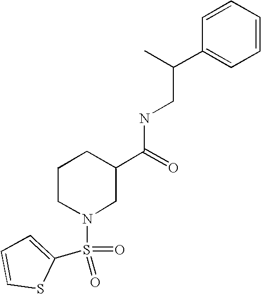 | Thiophene-2- sulfonyl chloride | 2-Phenyl- propylamine | 1-(Thiophene-2- sulfonyl)- piperidine-3- carboxylic acid (2-phenyl- propyl)-amide | 393 |
| 195 | 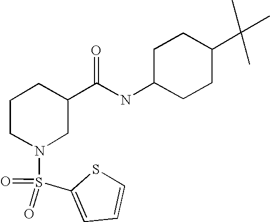 | Thiophene-2- sulfonyl chloride | 4-tert- Butylcyclohexyl- amine | 1-(Thiophene-2- sulfonyl)- piperidine-3- carboxylic acid (4-tert-butyl- cyclohexyl)- amide | 413 |
| 196 | 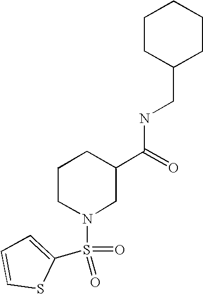 | Thiophene-2- sulfonyl chloride | Cyclohexyl- methylamine | 1-(Thiophene-2- sulfonyl)- piperidine-3- carboxylic acid cyclohexylmethyl- amide | 371 |
| 197 | 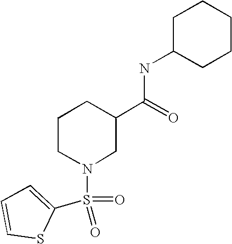 | Thiophene-2- sulfonyl chloride | Cyclohexylamine | 1-(Thiophene-2- sulfonyl)- piperidine-3- carboxylic acid cyclohexylamide | 357 |
| 198 | 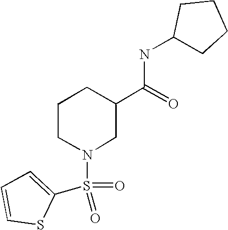 | Thiophene-2- sulfonyl chloride | Cyclopentamine | 1-(Thiophene-2- sulfonyl)- piperidine-3- carboxyhc acid cyclopentylamide | 343 |
| 199 | 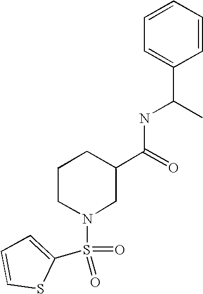 | Thiophene-2- sulfonyl chloride | dl-alpha- Methylbenzyl- amine | 1-(Thiophene-2- sulfonyl)- piperidine-3- carboxylic acid (1-phenyl- ethyl)-amide | 379 |
| 200 | 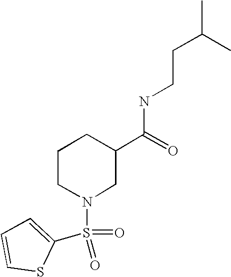 | Thiophene-2- sulfonyl chloride | Isoamylamine | 1-(Thiophene-2- sulfonyl)- piperidine-3- carboxylic acid (3-methyl- butyl)-amide | 345 |
| 201 | 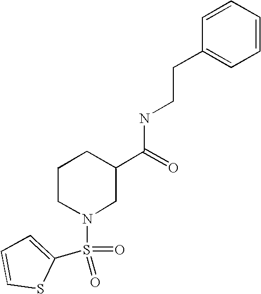 | Thiophene-2- sulfonyl chloride | Phenethylamine | 1-(Thiophene-2- sulfonyl)- piperidine-3- carboxylic acid phenethyl-amide | 379 |

### Example 202

### (rac)-1-(2-Chloro-benzenesulfonyl)-piperidine-3-carboxylic acid (3,5,7-trimethyl-adamantan-1-yl)-amide

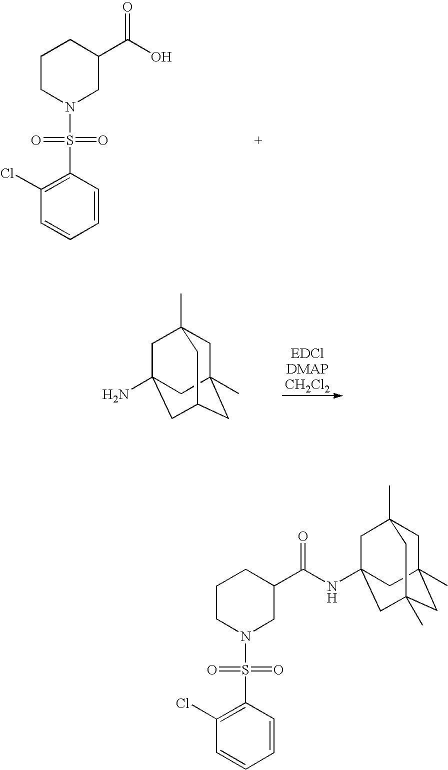

3,5,7-Trimethyl-1-adamantanamine (which can be prepared by the procedure described in J. G. Henkel and J. T. Hane J. Med Chem. 1982, 25, 51-56) (approx. 1.0 equiv) is added to a solution of (rac)-1-(2-chloro-benzenesulfonyl)-piperidine-3-carboxylic acid (of Intermediate A1; approx. 0.8 equiv), 1-hydroxybenzotriazole hydrate (1.1 equiv), N,N-dimethylaminopyridine (approx. 1.7 equiv), and 1-(3-dimethylaminopropyl)-3-ethylcarbodiimide hydrochloride (approx. 1.1 equiv) in dichloromethane (approx. 10 mL per equivalent). The solution is stirred for 24 h, and then diluted with dichloromethane, washed with 1 M HCl and then brine, dried (magnesium sulfate), filtered and evaporated. The crude product is purified by column chromatography, eluting with ethyl acetate/hexanes to give (rac)-1-(2-chloro-benzenesulfonyl)-piperidine-3-carboxylic acid (3,5,7-trimethyl-adamantan-1-yl)-amide.

### Example 203

### (rac)-1-(2-Chloro-benzenesulfonyl)-piperidine-3-carboxylic acid (3-hydroxy-adamantan-1-yl)-amide

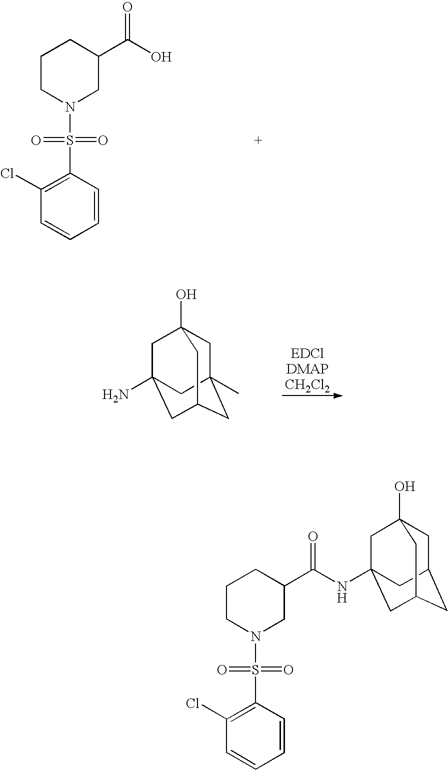

Amino-1-adamantanol (Aldrich Chemical Company, Inc., Milwaukee, Wisc.) (approx. 1.0 equiv) is added to a solution of (rac)-1-(2-chloro-benzenesulfonyl)-piperidine-3-carboxylic acid (of Intermediate A1; approx. 0.8 equiv), 1-hydroxybenzotriazole hydrate (1.1 equiv), N,N-dimethylaminopyridine (approx. 1.7 equiv), and 1-(3-dimethylaminopropyl)-3-ethylcarbodiimide hydrochloride (approx. 1.1 equiv) in dichloromethane (approx. 10 mL per equivalent). The solution is stirred for 24 h, and then diluted with dichloromethane, washed with 1 M HCl and then brine, dried (magnesium sulfate), filtered and evaporated. The crude product is purified by column chromatography, eluting with ethyl acetate/hexanes to give (rac)-1-(2-chloro-benzenesulfonyl)-piperidine-3-carboxylic acid (3-hydroxy-adamantan-1-yl)-amide.

### Example 204

### Testing of Compounds of the Invention in vitro

The in vitro inhibition of 11β-HSD1 by compounds of the present invention were demonstrated by means of the following test:

Purified human HSD1 was diluted in 50 mM Tris-HCl, 100 mM NaCl, 0.1 mg/ml BSA, 0.02% Lubrol, 20 mM MgCl2, 10 mM glucose 6-phosphate, 0.4 mM NADPH, 60 U/ml glucose 6-phosphate dehydrogenase to a concentration of 1.5 ug/ml (Enzyme Solution). Cortisone (100 uM) in DMSO was diluted to 1 uM with 50 mM Tris-HCl, 100 mM NaCl (Substrate Solution). Testing compounds (40 uM) in DMSO was diluted 3 fold in series in DMSO and further diluted 20 fold in Substrate Solution. Enzyme Solution (10 ul/well) was added into 384 well microtiter plates followed by diluted compound solutions (10 ul/well) and mixed well. Samples were then incubated at 370 C for 30 min. EDTA/biotin-cortisol solution (10 ul/well) in 28 mM EDTA, 100 nM biotin-cortisol, 50 mM Tris-HCl, 100 mM NaCl was then added followed by 5 ul/well of anti-cortisol antibody (3.2 ug/ml) in 50 mM Tris-HCl, 100 mM NaCl, 0.1 mg/ml BSA and the solution was incubated at 37 degrees for 30 min. Five ul per well of Eu-conjugated anti-mouse IgG (16 nM) and APC-conjugated streptavidin (160 nM) in 50 mM Tris-HCl, 100 mM NaCl, 0.1 mg/ml BSA was added and the solution was incubated at room temperature for 2 hours. Signals were quantitated by reading time-resolved fluorescence on a Victor 5 reader (Wallac).

Percent inhibition of HSD1 activity by an agent at various concentrations was calculated by the formula % Inhibition=100*[1−(Fs−Fb)/(Ft−Fb)], where:
Fs is the fluorescence signal of the sample which included the agent,
Fb is the fluorescence signal in the absence of HSD1 and agent,
Ft is the fluorescence signal in the presence of HSD1, but no agent.

The inhibitory activities of test compounds were determined by the IC₅₀s, or the concentration of compound that gave 50% inhibition.

The results of the in vitro inhibition of 11β-HSD1 by representative compounds of the present invention are shown in the following Table:

| Compound | hHSD1 IC₅₀ (μM) |
|---|---|
| Example 2 | 0.29 |
| Example 43 | 0.025 |
| Example 50 | 0.031 |
| Example 73 | 0.047 |
| Example 80 | 12 |
| Example 128 | 3 |
| Example 135 | 0.39 |
| Example 157 | 0.91 |
| Example 169 | 0.39 |
| Example 173 | 0.94 |
| Example 175 | 3 |
| Example 187 | 0.19 |

### Example 205

### Testing of Compounds of the Invention in vivo

The in vivo inhibition of 11β-HSD1 by compounds of the present invention can be demonstrated by means of the following test:

The compound of the invention is formulated in 7.5% Modified Gelatin in water and is administered IP at 100 mg/kg to mice (male C57B1/6J, age ˜97 Days). After 30 minutes, cortisone formulated in gelatin is administered by s.c. injection at 1 mg/kg. After a further 40 minutes, blood samples are taken from the mice and are analyzed using LC-MS for the concentrations of cortisone, cortisol, and drug.

Percent inhibition of HSD1 activity by the inhibitor is calculated by the following formula:
% Inhibition=100*[1−(Cᵢₙₕ/Cᵥₑₕ)]
where:
Cᵥₑₕ is the conversion of cortisone to cortisol when the animal is dosed with vehicle, and
Cᵢₙₕ is the conversion of cortisone to cortisol when the animal is dosed with inhibitor, where the conversion C is given by the formula C=[Cortisol]/([Cortisol]+[Cortisone]).

It is to be understood that the invention is not limited to the particular embodiments of the invention described above, as variations of the particular embodiments may be made and still fall within the scope of the appended claims.

What is claimed is:

1. A pharmaceutical composition comprising a therapeutically effective amount of a compound according to formula (I): 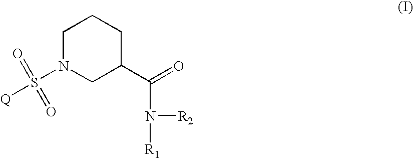
wherein
Q is unsubstituted phenyl,
substituted phenyl which is phenyl mono-, di-, or tri-substituted with a group independently selected from the group consisting of halogen, lower alkyl, —COOA, —CF3, —OA, —NC(═O)A, and phenyl,
unsubstituted heterocyclyl which is a 5- or 6-membered heteroaromatic ring which is connected by a ring carbon atom and which has from I to 3 hetero ring atoms selected from the group consisting of sulfur, nitrogen and oxygen,
substituted heterocyclyl which is heterocyclyl which is substituted with —COOA or halogen, naphthyl,
9- and 10-membered bicyclic unsaturated or partially unsaturated heterocyclyl which is connected by a ring carbon and which has from 1 to 3 hetero ring atoms selected from the group consisting of sulfur, nitrogen and oxygen,
substituted bicyclic heterocyclyl which is the 9- or 10-membered bicyclic heterocyclyl mono-,
bi- or tri-substituted with substituents selected from halogen or lower alkyl;
one of R₁ or R₂ is H and the other is selected from the group consisting of
lower alkyl,
a mono-substituted or unsubstituted saturated mono-, bi- or tri-cyclic 5 to 10 membered carbocyclic ring, wherein the mono-substituted carbocyclic ring is substituted with lower alkyl,
a bicyclic partially unsaturated 9- or 10-membered ring,
—CH₂B,
-D-phenyl or D-substituted phenyl, wherein D-substituted phenyl is D-phenyl in which the phenyl is mono- or di-substituted with —OA, halogen, or substituted or unsubstituted lower alkyl
-D-naphthyl,
-DE,
-DN(CH₃)n-phenyl,
-DNC(═O)A,
-DN(A)A,
-DOA;
or
R₁ and R₂, together with the N atom to which they are attached, form a substituted or unsubstituted ring Z, wherein Z is 6- or 7-membered monocyclic or 7- to 10-membered bicyclic saturated, partially unsaturated or unsaturated substituted or unsubstituted heterocyclic ring which contains the N atom to which R₁ and R₂ are attached, and optionally another hetero atom which is selected from N, O and S, wherein the substituted heterocyclic ring is mono- or di-substituted with lower alkyl or hydroxy or hydroxy-alkyl;
A is lower alkyl which has from 1 to 4 carbon atoms,
B is a 3- to 7-membered substituted or unsubstituted carbocyclic saturated ring,
D is the divalent form of A,
E is a 5- or 6-membered saturated, unsaturated or partially unsaturated heterocyclic ring having from 1 to 3 hetero atoms selected from the group consisting of S, N, and O,
n is zero or 1,
provided that where R₁ or R₂ is H and the other is lower alkyl, and where Q is monosubstituted in the para position with halogen, then the halogen is chloro,
provided that where R₁ or R₂ is H and the other is lower alkyl, and where Q is monosubstituted in the para position with lower alkyl, then the lower alkyl has from 1 to 3 carbon atoms,
provided that where R₁ or R₂ is H and the other is CH2B, and where Q is substituted phenyl wherein the phenyl ring is monosubstituted in the meta position with halogen, the halogen is not C₁,
provided that where R₁ or R₂ is H and the other is D-substituted phenyl in which D is —CH₂CH₂— and the phenyl is monosubstituted in the ortho position with F, and where Q is substituted phenyl wherein phenyl is monosubstituted with halogen, the halogen is not C₁ in the meta position,
provided that where R₁ or R₂ is H and the other is -D-substituted phenyl in which D is —CH₂— and the phenyl is monosubstituted with lower alkyl which is —CH₃ in the ortho position and where Q is substituted phenyl which is phenyl substituted with halogen, the halogen is not C₁ in the ortho position,
or a pharmaceutically acceptable salt thereof,
and a pharmaceutically acceptable carrier.

2. The pharmaceutical composition according to claim 1, wherein
Q is unsubstituted phenyl,
substituted phenyl which is phenyl mono-, di-, or tri-substituted with a group independently selected from the group consisting of halogen, lower alkyl, —COOA, —CF3, —OA, —NC(═O)A, and phenyl, and wherein
one of R₁ or R₂ is H and the other is selected from the group consisting of
lower alkyl,
a mono-substituted or unsubstituted saturated mono-, bi- or tri-cyclic 5 to 10 membered carbocyclic ring, wherein the mono-substituted carbocyclic ring is substituted with lower alkyl,
a bicyclic partially unsaturated 9- or 10-membered ring,
—CH₂B,
-D-phenyl or D-substituted phenyl, wherein D-substituted phenyl is D-phenyl in which the phenyl is mono- or di-substituted with —OA, halogen, or substituted or unsubstituted lower alkyl
-D-naphthyl,
-DE,
-DN(CH₃)n-phenyl,
-DNC(═O)A,
-DN(A)A, and
-DOA.

3. The pharmaceutical composition according to claim 1, wherein
Q is unsubstituted heterocyclyl which is a 5- or 6-membered heteroaromatic ring which is connected by a ring carbon atom and which has from 1 to 3 hetero ring atoms selected from the group consisting of sulfur, nitrogen and oxygen,
substituted heterocyclyl which is heterocyclyl which is substituted with —COOA or halogen, naphthyl, and wherein
one of R₁ or R₂ is H and the other is selected from the group consisting of
lower alkyl,
a mono-substituted or unsubstituted saturated mono-, bi- or tri-cyclic 5 to 10 membered carbocyclic ring, wherein the mono-substituted carbocyclic ring is substituted with lower alkyl,
a bicyclic partially unsaturated 9- or 10-membered ring,
—CH₂B,
-D-phenyl or D-substituted phenyl, wherein D-substituted phenyl is D-phenyl in which the phenyl is mono- or di-substituted with —OA, halogen, or substituted or unsubstituted lower alkyl
-D-naphthyl,
-DE,
-DN(CH₃)n-phenyl,
-DNC(═O)A,
-DN(A)A and
-DOA.

4. The pharmaceutical composition according to claim 1, wherein
Q is 9- and 10-membered bicyclic unsaturated or partially unsaturated heterocyclyl which is connected by a ring carbon and which has from I to 3 hetero ring atoms selected from the group consisting of sulfur, nitrogen and oxygen,
substituted bicyclic heterocyclyl which is the 9- or 10-membered bicyclic heterocyclyl mono-,
bi- or tri-substituted with substituents selected from halogen or lower alkyl; and wherein one of R₁ or R₂ is H and the other is selected from the group consisting of:
lower alkyl,
a mono-substituted or unsubstituted saturated mono-, bi- or tri-cyclic 5 to 10 membered carbocyclic ring, wherein the mono-substituted carbocyclic ring is substituted with lower alkyl,
a bicyclic partially unsaturated 9- or 10-membered ring,
—CH₂B,
-D-phenyl or D-substituted phenyl, wherein D-substituted phenyl is D-phenyl in which the phenyl is mono- or di-substituted with -OA, halogen, or substituted or unsubstituted lower alkyl
-D-naphthyl,
-DE,
-DN(CH₃)n-phenyl,
-DNC(═O)A,
-DN(A)A and
-DOA.

5. The pharmaceutical composition according to claim 1, wherein
Q is unsubstituted phenyl,
substituted phenyl which is phenyl mono-, di-, or tri-substituted with a group independently selected from the group consisting of halogen, lower alkyl, —COOA, —CF3, —OA, —NC(═O)A, and phenyl; and wherein
R₁ and R₂, together with the N atom to which they are attached, form a substituted or unsubstituted ring Z, wherein Z is 6- or 7-membered monocyclic or 7- to 10-membered bicyclic saturated, partially unsaturated or unsaturated substituted or unsubstituted heterocyclic ring which contains the N atom to which R₁ and R₂ are attached, and optionally another hetero atom which is selected from N, O and S, wherein the substituted heterocyclic ring is mono- or di-substituted with lower alkyl or hydroxy or hydroxy-alkyl.

6. The pharmaceutical composition according to claim 1, wherein
Q is unsubstituted heterocyclyl which is a 5- or 6-membered heteroaromatic ring which is connected by a ring carbon atom and which has from 1 to 3 hetero ring atoms selected from the group consisting of sulfur, nitrogen and oxygen,
substituted heterocyclyl which is heterocyclyl which is substituted with —COOA or halogen, naphthyl; and wherein
R₁ and R₂, together with the N atom to which they are attached, form a substituted or unsubstituted ring Z, wherein Z is 6- or 7-membered monocyclic or 7- to 10-membered bicyclic saturated, partially unsaturated or unsaturated substituted or unsubstituted heterocyclic ring which contains the N atom to which R₁ and R₂ are attached, and optionally another hetero atom which is selected from N, O and S, wherein the substituted heterocyclic ring is mono- or di-substituted with lower alkyl or hydroxy or hydroxy-alkyl.

7. The pharmaceutical composition according to claim 1, wherein
Q is 9- and 10-membered bicyclic unsaturated or partially unsaturated heterocyclyl which is connected by a ring carbon and which has from 1 to 3 hetero ring atoms selected from the group consisting of sulfur, nitrogen and oxygen,
substituted bicyclic heterocyclyl which is the 9- or 10-membered bicyclic heterocyclyl mono-,
bi- or tri-substituted with substituents selected from halogen or lower alkyl; and wherein
R₁ and R₂, together with the N atom to which they are attached, form a substituted or unsubstituted ring Z, wherein Z is 6- or 7-membered monocyclic or 7- to 10-membered bicyclic saturated, partially unsaturated or unsaturated substituted or unsubstituted heterocyclic ring which contains the N atom to which R₁ and R₂ are attached, and optionally another hetero atom which is selected from N, O and S, wherein the substituted heterocyclic ring is mono- or di-substituted with lower alkyl or hydroxy or hydroxy-alkyl.

8. The pharmaceutical composition according to claim 1, wherein said therapeutically effective amount of said compound is from about 10 mg to about 1000 mg per day.

9. The pharmaceutical composition according to claim 1, wherein halogen is Cl or F.

10. The pharmaceutical composition according to claim 1, wherein Q is unsubstituted thiophene, or heterocyclyl mono-substituted on a ring carbon with —COOCH3 or Cl.

11. The pharmaceutical composition according to claim 1, wherein Q is
9- or 10-membered bicyclic unsaturated or partially unsaturated heterocyclyl which is connected by a ring carbon and which has 1 or 2 hetero ring atoms selected from the group consisting of sulfur, nitrogen and oxygen, or
substituted bicyclic heterocyclyl which is the 9- or 10-membered bicyclic heterocyclyl with one or more substituents selected from halogen or lower alkyl.

12. The pharmaceutical composition according to claim 11, wherein Q is selected from the group consisting of

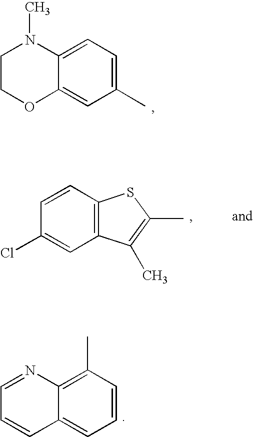

13. The pharmaceutical composition according to claim 1, wherein when one of R₁ or R₂ is H and the other is
a mono-substituted or unsubstituted saturated mono-, bi- or tri-cyclic 5 to 10 membered carbocyclic ring, said saturated carbocyclic ring is a five or six membered monocyclic ring or a 10 membered tricyclic ring, and wherein the mono-substituted carbocyclic ring is said saturated carbocyclic ring mono-substituted with lower alkyl.

14. The pharmaceutical composition according to claim 1, wherein when one of R₁ or R₂ is H and the other is
a bicyclic partially unsaturated 9- or 10-member ring, said ring is

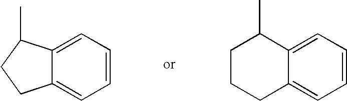

15. The pharmaceutical composition according to claim 1, wherein when one of R₁ or R₂ is H and the other is —CH₂B, B is a 3- or 6-membered carbocyclic saturated ring.

16. The pharmaceutical composition according to claim 1, wherein where one of R₁ or R₂ is H and the other is -D-phenyl or D-substituted phenyl, -D-phenyl is —CH₂CH(CH₃)-phenyl, —CH(CH₃)-phenyl, or —(CH₂)n-phenyl, and D-substituted phenyl is —CH(CH₃)-(fluoro-phenyl), —CH₂CH₂-(fluoro-phenyl), —CH₂-(trifluoromethyl-phenyl), —CH₂-(methyl-phenyl), —(CH₂)p-(chloro-phenyl), —(CH₂)p-(methoxy-phenyl), or —CH₂)p-(di-methoxy-phenyl),
wherein n is 1, 2, or 3, and
p is 1 or 2.

17. The pharmaceutical composition according to claim 1, wherein A is methyl.

18. The pharmaceutical composition according to claim 1, wherein where one of R₁ or R₂ is H and the other is DE, wherein D is —CH₂— or —CH₂CH₂—.

19. The pharmaceutical composition according to claim 1, wherein Z is selected from the group consisting of:

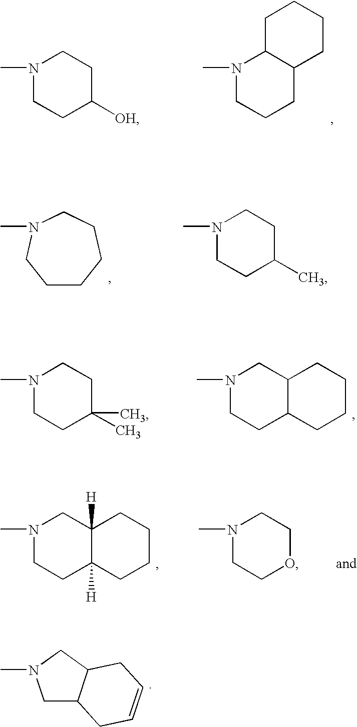

20. The pharmaceutical composition according to claim 1, wherein Q is phenyl substituted with chloro or methyl.

21. The pharmaceutical composition according to claim 20, wherein Q is phenyl substituted at the ortho position with chloro or methyl.

22. The pharmaceutical composition according to claim 21, wherein Q is monosubstituted.

23. The pharmaceutical composition according to claim 22, wherein Q is 2-methyl-phenyl.

24. The pharmaceutical composition according to claim 21, wherein Q is 2-chloro-phenyl.

25. The pharmaceutical composition according to claim 21, wherein Q is phenyl with two or three substituents selected from chloro or methyl.

26. The pharmaceutical composition according to claim 25, wherein Q is 2-chloro-6-methyl phenyl or 3-chloro-2-methyl-phenyl.

27. The pharmaceutical composition according to claim 1, wherein Q is unsubstituted phenyl.

28. The pharmaceutical composition according to claim 1, wherein Q is substituted or unsubstituted thiophenyl, or substituted or unsubstituted quinolinyl.

29. The pharmaceutical composition according to claim 28, wherein Q is unsubstituted thiophen-2-yl or unsubstituted quinolin-8-yl.

30. The pharmaceutical composition according to claim 1, wherein Q is phenyl substituted at the 4-position with halogen.

31. The pharmaceutical composition according to claim 30, wherein Q is 4-chloro-phenyl or 4-fluoro-phenyl

32. The pharmaceutical composition according to claim 13, wherein R₁ is hydrogen and R₂ is adamantan-1-yl.

33. The pharmaceutical composition according to claim 13, wherein R₁ is hydrogen and R₂ is cycloalkyl.

34. The pharmaceutical composition according to claim 19, wherein R₁, R₂ and the nitrogen to which they are attached is perhydroisoquinolin-2-yl.

35. The pharmaceutical composition according to claim 19, wherein R₁, R₂ and the nitrogen to which they are attached is perhydroquinolin-1-yl.

36. The pharmaceutical composition according to claim 18, wherein R₁ is hydrogen and R₂ is 2-(thiophen-2-yl)-ethyl.

37. The pharmaceutical composition according to claim 1, wherein said compound is: 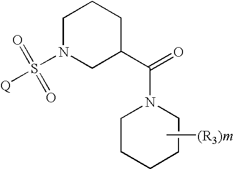
wherein R₃ is lower alkyl, and m is 1, 2, or 3.

38. The pharmaceutical composition according to claim 1, wherein R₁ is hydrogen and R₂ is D-naphthyl.

39. The pharmaceutical composition according to claim 1, wherein where one of R₁ or R₂ is H and the other is DE, E is selected from the group consisting of

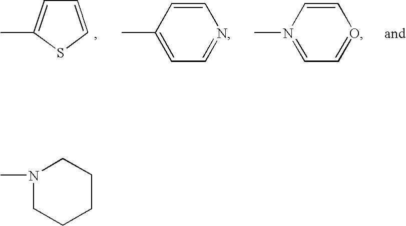

40. The pharmaceutical composition according to claim 1, wherein said compound is (3S)-1-(2-Chloro-benzenesulfonyl)-piperidine-3-carboxylic acid (3-methyl-butyl)-amide.

41. The pharmaceutical composition according to claim 1, wherein said compound is (3R)-1-(2-Chloro-benzenesulfonyl)-piperidine-3-carboxylic acid (3-methyl-butyl)-amide.

42. The pharmaceutical composition according to claim 1, wherein said compound is (rac)-[1-(2-Chloro-benzenesulfonyl)-piperidin-3-yl]-(octahydro-quinolin-1-yl)-methanone.

43. The pharmaceutical composition according to claim 1, wherein said compound is (rac)-Azepan-1-yl-[1-(2-chloro-benzenesulfonyl)-piperidin-3-yl]-methanone.

44. The pharmaceutical composition according to claim 1, wherein said compound is (3S)-1-(2,4-Dichloro-benzenesulfonyl)-piperidine-3-carboxylic acid cyclopentylamide.

45. The pharmaceutical composition according to claim 1, wherein said compound is 1-(2-Chloro-benzenesulfonyl)-piperidine-3-carboxylic acid cyclohexylamide.

46. The pharmaceutical composition according to claim 1, wherein said compound is 1-(2-Chloro-benzenesulfonyl)-piperidine-3-carboxylic acid cyclopentylamide.

47. The pharmaceutical composition according to claim 1, wherein said compound is 1-(Naphthalene-2-sulfonyl)-piperidine-3-carboxylic acid (3-phenyl-propyl)-amide.

48. The pharmaceutical composition according to claim 1, wherein said compound is 1-(3-Chloro-2-methyl-benzenesulfonyl)-piperidine-3-carboxylic acid cyclopropylmethyl-amide.

49. The pharmaceutical composition according to claim 1, wherein said compound is 1-(Quinoline-8-sulfonyl)-piperidine-3-carboxylic acid cyclohexylmethyl-amide.

50. The pharmaceutical composition according to claim 1, wherein said compound is 1-(Quinoline-8-sulfonyl)-piperidine-3-carboxylic acid cyclohexylamide.

51. The pharmaceutical composition according to claim 1, wherein said compound is 1-Benzenesulfonyl-piperidine-3-carboxylic acid (2-phenyl-propyl)-amide.

52. The pharmaceutical composition according to claim 1, wherein said compound is 1-Benzenesulfonyl-piperidine-3-carboxylic acid cyclohexylmethyl-amide.

53. The pharmaceutical composition according to claim 1, wherein said compound is 1-Benzenesulfonyl-piperidine-3-carboxylic acid cyclohexylamide.

54. A method for the treatment of type II diabetes in a patient in need thereof, comprising administering to said patient a therapeutically effective amount of a compound according to formula (I): 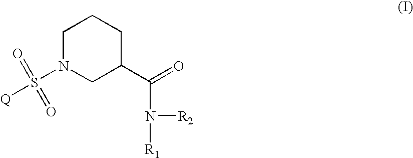
wherein:
Q is unsubstituted phenyl,
substituted phenyl which is phenyl mono-, di-, or tri-substituted with a group independently selected from the group consisting of halogen, lower alkyl, —COOA, —CF3, —OA, —NC(═O)A, and phenyl,
unsubstituted heterocyclyl which is a 5- or 6-membered heteroaromatic ring which is connected by a ring carbon atom and which has from 1 to 3 hetero ring atoms selected from the group consisting of sulfur, nitrogen and oxygen,
substituted heterocyclyl which is heterocyclyl which is substituted with —COOA or halogen, naphthyl,
9- and 10-membered bicyclic unsaturated or partially unsaturated heterocyclyl which is connected by a ring carbon and which has from 1 to 3 hetero ring atoms selected from the group consisting of sulfur, nitrogen and oxygen,
substituted bicyclic heterocyclyl which is the 9- or 10-membered bicyclic heterocyclyl mono-,
bi- or tri-substituted with substituents selected from halogen and lower alkyl;
one of R₁ or R₂ is H and the other is selected from the group consisting of
lower alkyl,
a mono-substituted or unsubstituted saturated mono-, bi- or tri-cyclic 5 to 10 membered carbocyclic ring, wherein the mono-substituted carbocyclic ring is substituted with lower alkyl,
a bicyclic partially unsaturated 9- or 10-membered ring,
—CH₂B,
-D-phenyl or D-substituted phenyl, wherein D-substituted phenyl is D-phenyl in which the phenyl is mono- or di-substituted with —OA, halogen, or substituted or unsubstituted lower alkyl
-D-naphthyl,
-DE,
-DN(CH₃)n-phenyl,
-DNC(═O)A,
-DN(A)A,
-DOA,
;or
R₁ and R₂, together with the N atom to which they are attached, form a substituted or unsubstituted ring Z, wherein Z is 6- or 7-membered monocyclic or 7- to 10-membered bicyclic saturated, partially unsaturated or unsaturated substituted or unsubstituted heterocyclic ring which contains the N atom to which R₁ and R₂ are attached, and optionally another hetero atom which is selected from N, O and S, wherein the substituted heterocyclic ring is mono- or di-substituted with lower alkyl or hydroxy or hydroxy-alkyl;
A is lower alkyl which has from 1 to 4 carbon atoms,
B is a 3- to 7-membered substituted or unsubstituted carbocyclic saturated ring,
D is the divalent form of A,
E is a 5- or 6-membered saturated, unsaturated or partially unsaturated heterocyclic ring having from 1 to 3 hetero atoms selected from the group consisting of S, N, and O,
n is zero or 1,
provided that where R₁ or R₂ is H and the other is lower alkyl, and where Q is monosubstituted in the para position with halogen, then the halogen is chloro,
provided that where R₁ or R₂ is H and the other is lower alkyl, and where Q is monosubstituted in the para position with lower alkyl, then the lower alkyl has from 1 to 3 carbon atoms,
provided that where R₁ or R₂ is H and the other is CH2B, and where Q is substituted phenyl wherein the phenyl ring is monosubstituted in the meta position with halogen, the halogen is not C₁,
provided that where R₁ or R₂ is H and the other is D-substituted phenyl in which D is —CH2CH2- and the phenyl is monosubstituted in the ortho position with F, and where Q is substituted phenyl wherein phenyl is monosubstituted with halogen, the halogen is not Cl in the meta position,
provided that where R₁ or R₂ is H and the other is -D-substituted phenyl in which D is —CH₂— and the phenyl is monosubstituted with lower alkyl which is —CH₃ in the ortho position and where Q is substituted phenyl which is phenyl substituted with halogen, the halogen is not Cl in the ortho position,
or a pharmaceutically acceptable salt thereof.
